(19) United States
(12) Patent Application Publication
(10) Pub. No.: US 2007/0042978 A1
(43) Pub. Date: Feb. 22, 2007

(54) Nf-hev compositions and methods of use

(76) Inventors: Jean-Philippe Girard, Rebigue (FR); Luc Aguilar, Escalquens (FR); Monique Erard, Toulouse (FR); Guttorm Haraldsen, Oslo (NO); Espen Baekkevold, Eiksmarka (NO); Marjan Veuger, Oslo (NO); Per Brandtzaeg, Oslo (NO)

Correspondence Address: KNOBBE MARTENS OLSON & BEAR LLP, 2040 MAIN STREET, FOURTEENTH FLOOR, IRVINE, CA, 92614, US

(21) Appl. No.: 10/539,527

(22) PCT Filed: Dec. 18, 2003
(86) PCT No.: PCT/IB03/06477

§ 371 Date: Jul. 10, 2006

Related U.S. Application Data
(60) Provisional application No. 60/435,827, filed on Dec. 19, 2002.

(51) Int. Cl. A61K 48/00; C07K 14/52; C12Q 1/68; C07H 21/04; C12P 21/06

(52) U.S. Cl. 514/44; 435/6; 435/69.1; 435/320.1; 435/325; 530/351; 536/23.5

(57) ABSTRACT

Aspects of the present invention relate to NF-HEV nuclear factor genes and polypeptides. Other aspects related to the use of NF-HEV nuclear factor genes and polypeptides. Other aspects related to the use of NF-HEV nuclear factor polynucleotides and polypeptides expressed in endothelial cells from chronically inflamed tissues, particularly in high endothelial venules endothelial cells (HEVECs) and endothelial cells from HEV-like vessels and small blood vessels, in connection with rheumatoid arthritis and Crohn's disease. Aspects of the invention also relates to drug screening assays for identifying compounds capable of modulating NF-HEV activity, wherein such compounds can be used in inhibiting or preventing chronic inflammation.

### FIELD OF THE INVENTION

The present invention relates to the field of biotechnology and medicine. In particular, the invention relates to NF-HEV and its role in inflammation and inflammatory diseases.

### BACKGROUND

Although all vascular endothelial cells (ECs) share certain common functions, it has become clear that considerable heterogeneity exists both structurally and functionally along the length of the vascular tree and in the microvascular beds of various organs. (Cines et al. (1998) Blood 91:3527-61; Garlanda and Dejana (1997) Arterioscler Thromb Vasc Biol 17:1193-202; Risau (1995) Faseb J 9:926-33; Simionescu et al. (1975) J Cell Biol 67:863-85) The structural heterogeneity of ECs is a perfect example of their adaptation to the unique demands of the actual tissue. ECs can either form a tight continuous monolayer in organs such as the brain or the lungs, where they perform important barrier functions. Alternatively, they can form a discontinuous layer with intercellular gaps or fenestrae in organs such as kidney, spleen or bone marrow, where rapid exchange of fluid, particles and cells takes place. (Risau (1995) Faseb J 9:926-33) The heterogeneity of ECs is also apparent at other levels. (Augustin et al. (1994) Bioessays 16:901-6; Garlanda and Dejana (1997) Arterioscler Thromb Vasc Biol 17:1193-202) For instance, several monoclonal antibodies (mAbs) and phage displayed-peptide sequences that distinguish among different types of ECs are available, (Augustin et al. (1994) Bioessays 16:901-6; Garlanda and Dejana (1997) Arterioscler Thromb Vasc Biol 17:1193-202; Pasqualini and Ruoslahti (1996) Nature 380:364-6; Rajotte et al. (1998) J Clin Invest 102:430-7) revealing antigenic differences between continuous and sinusoidal ECs, microvascular and large vessel ECs, as well as brain and lung ECs. (Page et al. (1992) Am J Pathol 141:673-83; Turner et al. (1987) Am J Clin Pathol 87:569-75) However, EC heterogeneity remains largely ill-defined at the molecular level and very few organ-specific EC markers have been described. (Cines et al. (1998) Blood 91:3527-61; Risau (1995) Faseb J 9:926-33)

One of the most striking examples of EC differentiation is the postcapillary high endothelial venules (HEVs) found in secondary organized lymphoid tissue (Girard and Springer (1995) Immunol Today 16:449-457; Kraal and Mebius (1997) Adv Immunol 65:347-95). Such vessels are particularly abundant in the T-cell zones that surround the B-cell follicles, and serve as entry sites for extravasating T and B lymphocytes. In contrast to the ECs from other vessels, the HEV endothelial cells or HEVECs have a plump almost cuboidal appearance, express specialized ligands for the lymphocyte homing receptor L-selectin, and are able to support extensive lymphocyte extravasion from blood. (Anderson and Anderson (1976) Immunology 31:731-48; Butcher and Picker (1996) Science 272:60-66; Girard and Springer (1995) Immunol Today 16:449-457; Kraal and Mebius (1997) Adv Immunol 65:347-95; Marchesi and Gowans (1964) Proc. R. Soc. Lond. B 159:283-290) At the ultrastructural level, HEVECs exhibit a prominent Golgi complex and glycocalix, abundant mitochondria closely associated with rough endoplasmic reticulum, and many ribosomes frequently found in polyribosomes clusters, revealing an intense biosynthetic activity generally not observed in ECs from other vessels. (Anderson et al. (1976) Immunology 31:455-73; Freemont and Jones (1983) J Anat 136:349-362; Wenk et al. (1974) J Ultrastruct Res 47:214-41) The specialized HEVECs also contain many membrane-bound vesicular structures, multivesicular bodies, Weibel-Palade bodies and a variety of dense bodies, indicating that they are involved in secretion. (Anderson et al. (1976) Immunology 31:455-73; Freemont and Jones (1983) J Anat 136:349-362; Wenk et al. (1974) J Ultrastruct Res 47:214-41) One of the major metabolic activity of HEVECs is the sulfation of L-selectin countereceptors. Sulfate residues have been shown to be the key for recognition of HEV sialomucins GlyCAM-1 and CD34 by L-selectin (Rosen (1999) Am J Pathol 155:1013-20) and MECA-79, an HEV-specific adhesion-blocking mAb (Michie et al. (1993) Am J Pathol 143:1688-1698; Streeter et al. (1988) J Cell Biol 107:1853-1862). Genes important for sulfation of L-selectin ligands in HEVs include the genes encoding PAPS synthetase, (Girard et al. (1998) Faseb J 12:603-12) a bifunctional enzyme which catalyzes synthesis of PAPS (3′-phosphoadenosine-5′-phosphosulfate), the activated sulfate donor used by all sulfotransferases, and L-selectin ligand N-acetyl-glucosamine-6-O-sulfotransferase (LSST) (Bistrup et al. (1999) J Cell Biol 145:899-910; Hemmerich et al. (2001) Immunity 15:237-47; Hiraoka et al. (1999) Immunity 11:79-89), which transfers sulfate from PAPS to HEV sialomucins. In addition to sulfated cell adhesion molecules (Rosen (1999) Am J Pathol 155:1013-20), HEVECs express high levels of secreted molecules such as the chemokine SLC/6Ckine, (Gunn et al. (1998) Proc Natl Acad Sci USA 95:258-63) which activates lymphocyte adhesiveness, and the SPARC-like antiadhesive matricellular protein hevin, (Girard and Springer (1995) Immunity 2:113-123) which may facilitate lymphocyte emigration by modulating EC-to-EC and EC-to-matrix adhesion. (Girard and Springer (1996) J Biol Chem 271:4511-4517)

Lymphocyte recruitment in HEVs depends on sequential multistep interactions between lymphocytes and HEVECs (von Andrian and Mackay (2000) N Engl J Med 343:1020-34), and is initiated by transient interactions between L-selectin on the lymphocyte microvilli and glycosylated and sulfated ligands on the HEV surface. This step is followed by chemokine activation of lymphocyte integrins via G protein-coupled chemokine receptors, resulting in firm adhesion mediated through interactions with their HEV ligands intercellular adhesion molecule (ICAM)-1/ICAM-2. Much progress has recently been made in the molecular understanding of this adhesion cascade, including the identification of the unique HEV-expressed sulfated carbohydrate ligands for L-selectin (Rosen (1999) Am J Pathol 155:1013-20) and the contribution by HEVECs to lymphocyte integrin activation by luminal presentation of endogenous or perivascularly derived chemokines (Baekkevold et al. (2001) J Exp Med 193:1105-12; Stein et al. (2000) J Exp Med 191:61-76).

HEV-like vessels also occur in chronically inflamed non-lymphoid tissue and may mediate aberrant lymphocyte influx at such sites. In rheumatoid arthritis, HEV-like vessels are seen close to the joint cavity, surrounded by dense lymphoid infiltrates (Freemont (1987) Ann Rheum Dis 46:924-928). Furthermore, in Crohn's disease and ulcerative colitis, collectively called inflammatory bowel disease (IBD), HEVs are found associated with extensive accumulations of lymphocytes (Salmi et al. (1994) Gastroenterology 106:596-605). Recently, HEV-like vessels were also found in nasal allergy and various chronic slin diseases, including lesions of cutaneous T-cell lymphomas (Farkas et al. (2001) Am J Pathol 159:237-43; Jahnsen et al. (2000) J Immunol 165:4062-8; Lechleitner et al. (1999) J Invest Dermatol 113:410-4). Finally, endothelium in rejecting heart transplants also exhibit HEV-like characteristics that correlate with the severity of the rejection (Toppila et al. (1999) Am J Pathol 155:1303-10). All these observations suggest that aberrant development of HEV-like vessels might mediate abnormal lymphocyte recruitment to the target tissue, thereby contributing to intensification and maintenance of chronic inflammation.

There is therefore a need for the identification of biological targets for the development of therapeutic molecules for the treatment of inflammation, particularly for inhibiting an inflammatory response in endothelial cells.

### SUMMARY OF THE INVENTION

Some embodiments of the present invention relate to use of a nuclear factor gene and protein specifically expressed in HEVEC and endothelial cells from chronically inflamed tissues. NF-HEV polypeptides can be used as targets for therapeutic intervention based on their role in promoting inflammation in endothelial cells. NF-HEV can also be involved in endothelial cell and more particularly HEVEC differentiation, as well as HEV-like vessel development. Provided herein is the characterization of NF-HEV, a nuclear factor expressed specifically in human endothelial cells from chronically inflamed tissues. Functional assays based on NF-HEV activity permits inflammation and HEV-like vessel formation to be examined. NF-HEV provides a valuable tool for modulating an endothelial cell's role in chronic inflammation as well as endothelial cell gene expression. NF-HEV can also provide a means for modulating endothelial cell, or preferably HEVEC, differentiation as well as HEV-like vessel development. NF-HEV therefore provides a valuable biological target for the inhibition of HEV-like vessel development or reducing HEV-like vessels already formed, thereby providing decreased adhesion of lymphocytes to HEVs, decreased lymphocyte extravasation to tissues and finally ameliorating or preventing inflammation, particularly chronic inflammation.

Some embodiments of the present invention concerns the role of NF-HEV polypeptides in modulating endothelial cell gene expression as well as in modulating endothelial cell phenotype, particularly phenotypic characteristics of HEVEC cells. As is further discussed herein, the NF-HEV polypeptides for use according to the present invention comprise NF-HEV peptides as well as biologically active fragments and variants thereof.

Further embodiments of the invention relate to recombinant vectors comprising any of the nucleic acid sequences described above, and in particular to recombinant vectors comprising a NF-HEV regulatory sequence or a sequence encoding a NF-HEV protein, as well as to cell hosts and transgenic non-human animals comprising said nucleic acid sequences or recombinant vectors.

Other embodiments of the present invention are also directed to methods for the screening of substances or molecules that inhibit the expression of the NF-HEV gene, as well as with methods for the screening of substances or molecules that interact with and/or inhibit the activity of a NF-HEV polypeptide.

In one aspect there is provided an expression cassette comprising a polynucleotide encoding a NF-HEV polypeptide. Preferably, such expression cassettes further comprises one or more regulatory sequences operably linked to said polynucleotide, which are capable of enhancing or otherwise modulating transcription and/or translation of said polynucleotide in a target cell, for example a mammalian cell. By way of illustration, in one embodiment, an expression cassette comprising a polynucleotide encoding a NF-HEV polypeptide operably linked to a promoter is provided. The promoter can be an inducible promoter or a constitutive promoter. The promoter can be heterologous to the NF-HEV coding sequence. Further, the promoter can be a ubiquitous promoter, for example a cytomegalovirus (CMV) promoter, rous sarcoma virus (RSV) promoter or human elongation factor (e.g., hEF-la) promoter, or it can be active only in certain tissues/cells. The expression cassette can be a viral expression construct for example, a retroviral vector, an adenoviral vector, an adeno-associated viral vector, a vaccina viral vector, a herpesviral vector, a polyoma viral construct, lentiviral vector or a Sindbis viral vector. The expression cassette can further comprise a second polynucleotide encoding a second polypeptide. The second, polypeptide can be, for example, a transcription factor, preferably an endothelial cell transcription factor.

In still a further aspect of the invention, there is provided a transformed host cell comprising a polynucleotide encoding a NF-HEV polypeptide and a promoter heterologous to the NF-HEV-encoding polynucleotide which promoter directs the expression of the NF-HEV polypeptide. The host cell can be prokaryotic or eukaryotic. In a related aspect of the invention, there is provided a method of using the transformed host cell and culturing it under conditions suitable for the expression of the NF-HEV polypeptide. In yet another aspect, there is provided a fusion protein comprising a NF-HEV protein or peptide fused to a second protein or peptide.

In yet a further and related aspect of the present invention, there is provided a method of modulating (e.g. stimulating or inhibiting) the expression of a gene in an endothelial cell. Modulating the expression of a gene in an endothelial cell can modulate an endothelial cell pro-inflammatory signaling pathway. In another aspect, the invention provides a method of converting a non-endothelial cell or non-HEVEC target cell, into an endothelial cell or a HEVEC, respectively, comprising introducing into the target cell an expression cassette that comprises a polynucleotide encoding a NF-HEV polypeptide as well as one or more regulatory sequences, for example, a promoter with or without enhancer sequences, such that regulatory sequences are active in the target cell and direct the expression of the polypeptide. The method can further comprise measuring endothelial cell or HEVEC lineage markers. In another aspect, the method involves introducing into the target cell a nucleic acid comprising a NF-HEV recognition element (e.g. a nucleotide sequence to which NF-HEV binds), said nucleic acid preferably being operably linked to a detectable polypeptide. In yet another aspect, the expression cassette can comprise one or more additional polynucleotides encoding one or more polypeptides, such as additional nuclear factors. By way of illustration, a second polypeptide can be a transcription factor, for example, an endothelial cell or HEVEC transcription factor. In a related aspect, expression of the additional polynucleotides can be under the control of the same regulatory sequences as the first polynucleotide or can be separately controlled by additional regulatory sequences. In another aspect of the present invention, the method further comprises introducing one or more additional expression cassettes into target cells separately from introduction of the NF-HEV expression cassette. By way of illustration, a second expression cassette comprising a polynucleotide encoding a second polypeptide and including a second promoter able to direct expression of the second polypeptide in the target cells can be delivered to the target cell using a separate gene delivering means from that used to introduce the NF-HEV expression cassette. Thus, for example, a first gene delivery vector comprising a NF-HEV expression cassette can be delivered simultaneously or contemporaneously with a second gene delivery vector comprising a second expression cassette. If desired, polypeptide expression can be measured, for example, by measuring transcription by RNA hybridization, RT-PCR or Western analysis.

In yet another aspect, there is provided a method of generating a modified endothelial cell, or more preferably a method of generating a modified HEVEC comprising introducing into a cell, preferably an endothelial cell, an expression cassette. The expression cassette comprises, for example, a polynucleotide encoding a NF-HEV polypeptide operatively linked to a promoter capable of directing of expression of the polypeptide. The promoter can be heterologous to the coding sequence and can be a ubiquitous (e.g., CMV) or a specific promoter (e.g., an alpha collagen promoter). The expression cassette can be introduced into the cell by any of a variety of means known to those of skill in the art. By way of illustration, lipid-based vectors (e.g., liposomes), viral vectors (e.g., retroviral vectors; vaccinia viral vectors, herpesviral vectors, polyoma viral constructs, lentiviral vectors or Sindbis viral vectors), or other macromolecular complexes capable of mediating delivery of the polynucleotide to the target cell, can be employed.

In a further aspect the gene delivery vector can be modified, for example by means known to those of skill in the art, to target one or more specific cell types. The expression cassette can also comprise a selectable marker, e.g., an immunologic marker. The expression cassette can further comprise a second polynucleotide encoding a second polypeptide, such as endothelial cell or HEVEC-active transcription factor. Such a second polynucleotide can be under control of a second promoter or the same promoter as the first polynucleotide. Alternatively, an internal ribosomal entry site (IRES) can be employed between the two transgenes to permit expression of the second transgene.

In a further aspect of the present invention, there is provided a method of modulating the expression of a gene in an endothelial cell comprising inhibiting the function or expression of NF-HEV. Preferably said method causes the decreased expression of a pro-inflammatory protein in an endothelial cell. In another aspect the invention provides a method for modulating endothelial cell phenotype, preferably HEVEC cell phenotype, or preferably reducing or preventing the development of BEV-like vessels, comprising inhibiting the function of NF-HEV. In one aspect, NF-HEV function can be reduced in a post-mitotic endothelial cell or HEVEC. Inhibiting can also comprise providing antisense nucleic acid that inhibits transcription or translation of a NF-HEV mRNA, or small interfering RNAs that induces degradation of a NF-HEV mRNA. The antisense nucleic acid or small interfering RNAs can be provided by introducing an expression cassette encoding NF-HEV antisense RNA or small interfering RNAs.

As further discussed herein, chronic inflammatory disorders typically involve development of HEV-like vessels. This development can be the result of the activities of cells, especially non-HEVEC cells which differentiate into HEVEC or HEV-like vessel cells in the region of disease. In preferred aspects of the present invention, compositions and methods are provided that alleviate the deleterious inflammation potentiating activities of such HEVEC cells or cells from HEV-like vessels by modulating the phenotype of said cells.

In some embodiments, the compositions and methods can be used not only to alleviate or prevent the deleterious pro-inflammatory activities of the target cell population (in this case endothelial cells such as HEVECs or cells from HEV-like vessels) but also to stimulate the target cells to engage in one or more functions typical of endothelial cells not involved in inflammation, thereby reducing inflammation or inflammatory potential in the diseased region. By way of illustration, lymphocyte cells typically bind and extravasate from HEV or HEV-like vessels, thereby resulting in chronic inflammation and possibly related tissue damage. Introduction of a composition in accordance herewith into such HEV-like vessels or small blood vessels capable of differentiating thereinto can prevent those cells from engaging in such deleterious activity.

According to one aspect of the invention, modulating inflammation comprises modulating, preferably inhibiting, the transcription of a gene in an endothelial cell. Preferably said gene encodes a polypeptide involved in a pro-inflammatory pathway. In a some aspects, modulating HEVEC phenotype comprises modulating transcription of a gene involved in determining (e.g. inducing differentiation of or maintaining) the HEVEC phenotype. Some embodiments of the invention involves methods of detecting or assessing NF-HEV activity comprising detecting the expression or transcription of one or a plurality of endothelial markers or HEVEC lineage markers. Detecting the expression or transcription of one or a plurality of endothelial markers or HEVEC lineage markers can include detecting an mRNA or protein known to be expressed in an endothelial cell, or alternatively can include detecting a polypeptide encoded by a polynucleotide operably linked to a transcriptional regulatory sequence known to be active in an endothelial cell. Other methods of detecting the expression of transcription of one or a plurality of endothelial markers are also contemplated.

In one aspect the method comprises (a) introducing to a cell an expression cassette comprising a polynucleotide encoding a NF-HEV polypeptide operatively linked to a promoter capable of directing of expression of the polypeptide; and (b) detecting expression or transcription from an endothelial cell regulatory sequence (e.g. detecting a polypeptide under the regulatory control of a regulatory sequence active in an endothelial cell). The method can also comprise (a) introducing to a cell an expression cassette comprising a polynucleotide encoding a NF-HEV polypeptide operatively linked to a promoter capable of directing of expression of the polypeptide; and (b) detecting expression or transcription of an endothelial cell marker, preferably a HEVEC marker. In other aspects, as further described in the section entitled “Drug Screening Assays”, the invention comprises: (a) introducing to the cell an inhibitor of an NF-HEV polypeptide; (b) optionally, providing to the cell a NF-HEV polypeptide; (c) optionally, providing to the cell a polynucleotide encoding an additional polypeptide factor, preferably a transcription factor; and (d) detecting expression or transcription of an endothelial cell marker, preferably a HEVEC marker. In other aspects, detecting the expression of transcription of an endothelial cell marker comprises detecting expression or transcription from an endothelial cell regulatory sequence.

In some embodiments, the screening method comprises: (a) introducing to a cell an inhibitor of an NF-HEV polypeptide; (b) optionally, introducing to a cell an expression cassette comprising a polynucleotide encoding a NF-HEV polypeptide operatively linked to a promoter capable of directing of expression of the polypeptide; (c) optionally, introducing to a cell an expression cassette comprising a polynucleotide encoding an additional polypeptide factor, preferably a transcription factor, said polynucleotide operatively linked to a promoter capable of directing of expression of the polypeptide; and (d) detecting expression or transcription of an endothelial cell marker, or an HEVEC marker. In some embodiments, the endothelial cell or HEVEC marker is a lineage marker. In one aspect of the methods, the expression of a endothelial cell or HEVEC marker mRNA or polypeptide is detected. In another example, the method comprises introducing to the cell an expression cassette comprising a polynucleotide encoding a detectable polypeptide operatively linked to a transcriptional regulatory sequence of a gene encoding an endothelial cell or HEVEC marker.

In still another aspect, there is provided a non-human transgenic animal, e.g., a mouse, comprising an expression cassette. The expression cassette comprises a polynucleotide encoding a NF-HEV peptide or protein and a promoter operably linked thereto which promoter can be heterologous to the NF-HEV peptide or protein encoding region. The promoter can be a constitutive or an inducible promoter. The expression cassette may further comprise selectable marker(s). In a related aspect of the present invention, the non-human transgenic animal may comprise a defective germ-line NF-HEV allele or two defective germ-line NF-HEV alleles.

In a further aspect of the invention, there are provided methods of ameliorating the symptoms associated with and of treating an inflammatory disorder, such as rheumatoid arthritis, Crohn's disease or inflammatory bowel disorder. The methods comprise administering to an animal suffering from an inflammatory disorder a compound capable of inhibiting NF-HEV activity. In some aspects there are provided a method of alleviating one or more symptoms of an inflammatory disorder comprising inhibiting the function of NF-HEV in postmitotic endothelial cells or HEVECs in the subject.

An additional aspect of the present invention is to provide compositions and methods for the identification of downstream target genes of NF-HEV polypeptides.

A gene delivery vector, for example an adenoviral vector, can be employed to deliver a NF-HEV gene to isolated endothelial cells thereby permitting over-expression of the NF-HEV polypeptide. Differences in gene profiling between control (i.e., nontransfected) endothelial cells and transfected (i.e., NF-HEV-overexpressing) endothelial cells can then be assessed by standard methods, such as differential display and microarray (e.g., gene chip) technology. Genes that are activated by NF-HEV in endothelial cells can subsequently be evaluated as potential therapeutics, for example, using bioinformatics techniques.

In yet another aspect of the present invention, there is provided a method of screening for a candidate substance for an effect on NF-HEV regulation of endothelial cell or HEVEC gene expression or endothelial cell or HEVEC development, said method comprising: (a) providing NF-HEV and optionally one ore more further HEVEC factors (e.g. transcription factor) to a cell; (b) admixing NF-HEV and optionally said further HEVEC factor(s) in the presence of the candidate substance; and (c) measuring the effect of the candidate substance on the expression of an endothelial cell or HEVEC marker, wherein a difference in the expression of the endothelial cell or HEVEC marker, as compared to an untreated cell, indicates that the candidate substance effects NF-HEV regulation of endothelial cell or HEVEC gene expression or development.

Exemplary cells include endothelial cells such HEVECs, which can be located in an animal. The modulator can increase or decrease the expression of the HEVEC lineage marker. Any suitable lineage marker can be used. Examples of HEVEC lineage marker include the L-selectin ligand N-acetyl-glucosamine-6-O-sulfotransferase (LSST) (Bistrup et al. (1999) J Cell Biol 145:899-910; Hemmerich et al. (2001) Immunity 15:237-47; Hiraoka et al. (1999) Immunity 11:79-89), and the HEV-specific MECA-79 sulfated epitope (Michie et al. (1993) Am J Pathol 143:1688-1698; Streeter et al. (1988) J Cell Biol 107:1853-1862), the fucosyltransferase FucTVII (Maly et al. (1996) Cell 86:643-653; Smith et al. (1996) J Biol Chem 271:8250-8259) and the HECA-452-fucosylated epitope (Duijvestijn et al. (1988) Am J Path 130:147-155), the chemokine CCL21 (SLC/6Ckine/TCA-4/exodus-2) (Gunn et al. (1998) Proc Natl Acad Sci USA 95:258-63) and the SPARC-like antiadhesive matricellular protein hevin (Girard and Springer (1995) Immunity 2:113-123; Girard and Springer (1996) J Biol Chem 271:4511-4517), The method of measuring the expression of the endothelial cell or HEVEC markers can include, but is not limited to, RNA hybridization, RT-PCR, immunologic detection, ELISA or immunohistochemistry.

In still yet another aspect of the invention, there is provided a method of screening for a modulator of NF-HEV expression comprising: (a) providing a cell that expresses a NF-HEV polypeptide; (b) contacting the NF-HEV polypeptide with a candidate substance; and (c) measuring the expression of NF-HEV, wherein a difference in NF-HEV expression, indicates that the candidate substance is a modulator of NF-HEV expression. In some embodiments, the modulator is a pharmaceutical composition. In some embodiments, the modulator enhances or inhibits NF-HEV expression.

Other aspects of the present invention are described with reference to the numbered paragraphs below:

1. A method of modulating the level or activity of a chemokine, said method comprising modulating in an endothelial cell the level or activity of the NF-HEV polypeptide or a biologically active fragment thereof, thereby modulating the level or activity of said chemokine.

2. The method of Paragraph 1, wherein the level or activity of said NF-HEV polypeptide or a biologically active fragment thereof is modulated by altering the expression of a nucleic acid encoding said NF-HEV polypeptide or a biologically active fragment thereof in said cell.

The method of Paragraph 1, wherein the level or activity of said NF-HEV polypeptide or a biologically active fragment thereof is modulated by providing said cell with a compound.

4. The method of Paragraph 1, wherein said endothelial cell is an HEVEC.

5. Then method of Paragraph 4, wherein said cell is a mammalian cell.

6. The method of Paragraph 5, wherein said HEVEC cell is a human cell.

7. The method of Paragraph 1, wherein in said chemoline is a pro-inflammatory chemoline.

8. The method of Paragraph 7, wherein said pro-inflammatory chemokine is selected from the group consisting of XCL1/GROα, CXCL2/GROβ, CXCL6, CXCL8/IL8 and CCL2/MCP1.

9. The method of Paragraph 7, wherein the level or activity of said pro-inflammatory chemokine is reduced.

10. The method of Paragraph 1, wherein the level or activity of said NF-HEV polypeptide or a biologically active fragment thereof is reduced.

11. The method of Paragraph 10, wherein the level or activity of said NF-HEV polypeptide or a biologically active fragment thereof is reduced by reducing the expression of a nucleic acid encoding said NF-HEV polypeptide or a biologically active fragment thereof in said cell.

12. The method of Paragraph 11, wherein the expression of a nucleic acid encoding said NF-HEV polypeptide or a biologically active fragment thereof is reduced by providing an antisense nucleic acid complementary to at least a portion of said NF-HEV polypeptide or a biologically active fragment thereof.

13. The method of Paragraph 10, wherein the level or activity of said NF-HEV polypeptide or a biologically active fragment thereof is reduced by reducing the activity or level of a pro-inflammatory cytokine.

14. A method of reducing the level or activity of a chemokine, said method comprising reducing in a cell the level or activity of the NF-HEV polypeptide or a biologically active fragment thereof, thereby reducing the level or activity of a chemokine.

15. The method of Paragraph 14, wherein reducing the level or activity of the NF-HEV polypeptide or a biologically active fragment thereof does not include reducing the level or activity of a pro-inflammatory cytokine.

16. The method of Paragraph 14, wherein the level or activity of the NF-HEV polypeptide or a biologically active fragment thereof is reduced by providing to said cell a compound that reduces the level or activity of the NF-HEV polypeptide or a biologically active fragment thereof.

17. The method of Paragraph 16, wherein the compound is an antisense nucleic acid that is complementary to at least a portion of a nucleic acid encoding NF-HEV.

18. The method of Paragraph 16, wherein the compound is an siRNA specific for at least a portion of a nucleic acid encoding NF-HEV.

19. The method of Paragraph 14, wherein in said chemokine is a pro-inflammatory chemokine.

20. The method of Paragraph 19, wherein said pro-inflammatory chemokine is selected from the group consisting of XCL1/GROα, CXCL2/GROβ, CXCL6, CXCL8/L8 and CCL2/MCP1.

21. The method of Paragraph 14, wherein the cell is an endothelial cell.

22. The method of Paragraph 21, wherein the cell is a HEVEC cell.

23. A method of ameliorating symptoms of a condition associated with inflammation, said method comprising identifying a subject having symptoms of a condition associated with inflammation; and modulating in said subject the level or activity of the NF-HEV polypeptide or a biologically active fragment thereof, thereby ameliorating symptoms of a condition associated with inflammation.

24. The method of Paragraph 23, wherein the level or activity of said NF-HEV polypeptide or a biologically active fragment thereof is modulated by altering the expression of a nucleic acid encoding said NF-HEV polypeptide or a biologically active fragment thereof.

25. The method of Paragraph 23, wherein the level or activity of said NF-HEV polypeptide or a biologically active fragment thereof is modulated by administering a compound to said subject.

26. The method of Paragraph 23, wherein modulating the level or activity of said NF-HEV polypeptide or a biologically active fragment thereof modulates the level or activity of a pro-inflammatory chemokine.

27. The method of Paragraph 26, wherein said pro-inflammatory chemokine is selected from the group consisting of XCL1/GROα, CXCL2/GROβ, CXCL6, CXCL8/IL8 and CCL2/MCP1.

28. The method of Paragraph 26, wherein the level or activity of said pro-inflammatory chemokine is reduced.

29. The method of Paragraph 23, wherein the level or activity of said NF-HEV polypeptide or a biologically active fragment thereof is reduced.

30. The method of Paragraph 29, wherein the level or activity of said NF-HEV polypeptide or a biologically active fragment thereof is reduced by reducing the expression of a nucleic acid encoding said NF-HEV polypeptide or a biologically active fragment thereof.

31. The method of Paragraph 30, wherein the expression of a nucleic acid encoding said NF-HEV polypeptide or a biologically active fragment thereof is reduced by providing an antisense nucleic acid complementary to at least a portion of said NF-HEV polypeptide or a biologically active fragment thereof.

32. The method of Paragraph 29, wherein the level or activity of said NF-HEV polypeptide or a biologically active fragment thereof is reduced by reducing the activity or level of a pro-inflammatory cytokine.

33. A method of ameliorating the symptoms of a condition associated with inflammation, said method comprising modulating the level of transcription of at least one promoter responsive to an NF-HEV polypeptide or biologically active fragment thereof.

The method of Paragraph 33, wherein the level of transcription of said at least one promoter responsive to an NF-HEV polypeptide or biologically active fragment thereof is reduced.

The method of Paragraph 33, wherein modulating the level or activity of said promoter modulates the level or activity of a pro-inflammatory chemokine.

36. The method of Paragraph 35, wherein said pro-inflammatory chemokine is selected from the group consisting of XCL1/GROα, CXCL2/GROβ, CXCL6, CXCL8/IL8 and CCL2/MCP1.

37. The method of Paragraph 35, wherein the level or activity of said pro-inflammatory chemoline is reduced.

38. A nucleic acid selected from the group consisting of: (i) a nucleic acid molecule encoding a polypeptide comprising an amino acid sequence selected from the group of sequences consisting of SEQ ID NOs: 4-5; (ii) a nucleic acid molecule comprising a nucleic acid sequence selected from the group of sequences consisting of SEQ ID NOs: 1-2, or a sequence complementary thereto; (iii) a nucleic acid molecule the complementary strand of which hybridizes under stringent conditions to a nucleic acid as defined in (i) and (ii); and (iv) a nucleic acid the sequence of which is degenerate as a result of the genetic code to a sequence of a nucleic acid as defined in (i), (ii) and (iii).

39. The nucleic acid of Paragraph 38, wherein said nucleic acid is operably linked to a promoter.

40. An expression cassette comprising the nucleic acid of Paragraph 39.

41. A host cell comprising the expression cassette of Paragraph 40.

42. An isolated nucleic acid comprising a nucleotide sequence encoding: i) a polypeptide comprising an amino acid sequence having at least about 80% identity to a sequence selected from the group consisting of the polypeptides of SEQ ID NOs: 4-5, and the polypeptides encoded by the nucleic acid of SEQ ID NOs: 1-2; or ii) a biologically active fragment of said polypeptide.

43. The nucleic acid of Paragraph 42, wherein said polypeptide comprises an amino acid sequence selected from the group consisting of the sequences shown as SEQ ID NOs: 4-5 and the polypeptides encoded by the nucleic acid of SEQ ID NOs: 1-2.

44. A method of making a NF-HEV polypeptide, said method comprising: a) providing a population of host cells comprising a nucleic acid encoding said NF-HEV protein having an amino acid sequence selected from the group consisting of SEQ ID NOs: 4-5 and sequence having at least 80% amino acid identity to SEQ ID NOs: 4-5; and b) culturing said population of host cells under conditions conducive to the expression of said recombinant nucleic acid, whereby said polypeptide is produced within said population of host cells.

45. The method of Paragraph 44, further comprising purifying said polypeptide from said population of cells.

46. An isolated nucleic acid, said nucleic acid comprising a nucleotide sequence having at least about 80% identity over at least about 100 nucleotides to a sequence selected from the group consisting of SEQ ID NOs: 1-2 and sequences complementary to SEQ ID NOs: 1-2.

47. The nucleic acid of Paragraph 46, wherein said nucleic acid hybridizes under stringent conditions to a nucleic acid having a nucleotide sequence selected from the group consisting of SEQ ID NOs: 1-2 and sequences complementary to SEQ ID NOs: 1-2.

48. The nucleic acid of Paragraph 46, wherein identity is determined using an algorithm selected from the group consisting of NBLAST with the parameters score=100 and wordlength=12, Gapped BLAST with the default parameters of NBLAST, and BLAST with the default parameters of NBLAST.

49. A biologically active NF-HEV polypeptide encoded by the nucleic acid of Paragraph 38 or 42.

50. A biologically active isolated NF-HEV polypeptide or fragment thereof, said polypeptide comprising an amino acid sequence having at least about 80% amino acid sequence identity to a sequence selected from the group consisting of SEQ ID NOs: 4-5.

51. The polypeptide of Paragraph 50, wherein said polypeptide is selectively bound by an antibody raised against an antigenic polypeptide, or antigenic fragment thereof, said antigenic polypeptide comprising a polypeptide selected from the group consisting of SEQ ID NOs: 4-5.

52. The polypeptide of Paragraph 50, wherein said polypeptide comprises a polypeptide selected from the group consisting of SEQ ID NOs: 4-5.

53. An antibody that selectively binds to the polypeptide of Paragraph 49.

54. A method of determining whether a NF-HEV nucleic acid or polypeptide is expressed within a biological sample, said method comprising the steps of: a) contacting said biological sample with a polynucleotide that hybridizes under stringent conditions to a nucleic acid of Paragraph 38 or a detectable polypeptide that selectively binds to the polypeptide of Paragraph 50 or Paragraph 52; and b) detecting the presence or absence of hybridization between said polynucleotide and an RNA species within said sample, or the presence or absence of binding of said detectable polypeptide to a polypeptide within said sample, wherein a detection of said hybridization or of said binding indicates that said NF-HEV is expressed within said sample.

55. The method of Paragraph 54, wherein said polynucleotide is a primer, and wherein said hybridization is detected by detecting the presence of an amplification product comprising said primer sequence.

56. The method of Paragraph 54, wherein said detectable polypeptide is an antibody.

57. A method of determining whether a mammal has an elevated or reduced level of NF-HEV expression, said method comprising the steps of: a) providing a biological sample from said mammal; and b) comparing the amount of a NF-HEV polypeptide of Paragraph 50 or Paragraph 52 or of a NF-HEV RNA species encoding a polypeptide of Paragraph 50 within said biological sample with a level detected in or expected from a control sample, wherein an increased amount of said NF-HEV polypeptide or said NF-HEV RNA species within said biological sample compared to said level detected in or expected from said control sample indicates that said mammal has an elevated level of NF-HEV expression, and wherein a decreased amount of said NF-HEV polypeptide or said NF-HEV RNA species within said biological sample compared to said level detected in or expected from said control sample indicates that said mammal has a reduced level of NF-HEV expression.

58. A method of identifying a candidate inhibitor of a NF-HEV polypeptide, said method comprising: a) contacting a NF-HEV polypeptide according to Paragraph 50 or Paragraph 52 or a fragment thereof which comprises a contiguous span of at least 6 contiguous amino acids of the polypeptide according to Paragraph 50 or Paragraph 52 with a test compound; and b) determining whether said compound selectively binds to said polypeptide, wherein a determination that said compound selectively binds to said polypeptide indicates that said compound is a candidate inhibitor of said polypeptide.

59. The method of Paragraph 58, wherein a determination that said compound selectively binds to said polypeptide indicates that said compound is a candidate compound for the treatment of a chronic inflammatory disorder.

60. A method of identifying a candidate inhibitor of a NF-HEV polypeptide of Paragraph 50 or Paragraph 52 or a fragment comprising a contiguous span of at least 6 contiguous amino acids of the polypeptide according to Paragraph 50 or Paragraph 52, said method comprising: a) contacting said polypeptide with a test compound; and b) determining whether said compound selectively inhibits at least one activity of said polypeptide, wherein a determination that said compound selectively inhibits at least one activity of said polypeptide indicates that said compound is a candidate inhibitor of said polypeptide.

61. The method of Paragraph 60, wherein a determination that said compound selectively inhibits said at least one biological activity of said polypeptide indicates that said compound is a candidate compound for the treatment of a chronic inflammatory disorder.

62. A method of identifying a candidate NF-HEV inhibitor, said method comprising: a) providing a cell comprising a NF-HEV polypeptide or a fragment comprising at least 6 consecutive amino acids thereof; b) contacting said cell with a test compound; and c) determining whether said compound selectively inhibits at least one NF-HEV activity, wherein a determination that said compound selectively inhibits activity of said polypeptide indicates that said compound is a candidate inhibitor of said polypeptide.

63. The method of Paragraph 62, wherein a determination that said compound selectively inhibits said at least one biological activity of said polypeptide indicates that said compound is a candidate compound for the treatment of a chronic inflammatory disorder.

64. The method of Paragraph 62, wherein step (a) comprises introducing a nucleic acid comprising the nucleotide sequence encoding said NF-HEV polypeptide according to any one of Paragraphs 38, 39, 42 or 43 into said cell.

65. The method of any of Paragraphs 58 to 64, wherein said NF-HEV activity comprises modulating gene expression in an endothelial cell.

66. The method of any of Paragraphs 58 to 64, wherein said NF-HEV activity comprises modulating the inflammatory potential of an endothelial cell.

67. The method of any of Paragraphs 58 to 64, wherein said NF-HEV activity comprises modulating the phenotype of an endothelial cell.

68. The method of any of Paragraphs 58 to 64, wherein said NF-HEV activity comprises regulating HEV-like vessel development or maintenance.

69. The method of any of Paragraphs 58 to 64, wherein said NF-HEV activity comprises modulating the differentiation or proliferation of an endothelial cell.

70. The method of any of Paragraphs 58 to 64, wherein said NF-HEV polypeptide or fragment thereof comprises a homeodomain-like helix-turn-helix (HTH) DNA-binding domain.

71. The method of any of Paragraphs 58 to 64, wherein said NF-HEV polypeptide or fragment thereof comprises the amino acid sequence of positions 61 to 78 of SEQ ID NO: 1 or 63 to 80 of SEQ ID NO: 2.

72. A polynucleotide according to any one of Paragraphs 38, 39, 42 or 43 attached to a solid support.

73. An array of polynucleotides comprising at least one polynucleotide according to Paragraph 72.

74. An array according to Paragraph 72, wherein said array is addressable.

75. A polynucleotide according to any one of Paragraphs 38, 39, 42 or 43 further comprising a label.

76. A viral composition comprising a recombinant viral vector encoding a NF-HEV protein according to Paragraphs 50 or 52.

77. The composition of Paragraph 76, wherein said recombinant viral vector is selected from the group consisting of an adenoviral, adeno-associated viral, retroviral, herpes viral, papilloma viral, and hepatitus B viral vector.

A method of modulating endothelial cell differentiation comprising modulating the activity of the NF-HEV protein.

79. A method of modulating endothelial cell differentiation comprising modulating the activity of the NF-HEV protein.

80. A method of inducing the differentiation of an endothelial cell comprising contacting a cell with a NF-HEV polypeptide or with a nucleic acid encoding a NF-HEV polypeptide.

81. The method of Paragraph 80 comprising inducing the differentiation of a HEVEC cell.

82. A method according to Paragraphs 80 or 81, comprising contacting said subject with a recombinant vector encoding a NF-HEV protein according to any one of Paragraphs 43 or 45 operably linked to a promoter that functions in said cell.

83. A method of modulating extravasation of lymphocytes in an individual comprising modulating the activity of the NF-HEV protein in said individual.

84. A method of reducing inflammation in an individual comprising inhibiting the activity of the NF-HEV protein in said individual.

85. A method of increasing extravasation of lymphocytes in an individual comprising increasing the activity of the NF-HEV protein in said individual.

86. A nucleic acid comprising a contiguous span of at least 20 nucleotides of a sequence selected from the group consisting of SEQ ID NOs: 1-2, and sequences complementary to SEQ D NOs: 1-2.

87. A method of identifying a candidate activator of a NF-HEV polypeptide, said method comprising: a) contacting a NF-HEV polypeptide according to Paragraph 50 or Paragraph 52 or a fragment comprising a contiguous span of at least 6 contiguous amino acids of a polypeptide according to Paragraph 50 or Paragraph 52 with a test compound; and b) determining whether said compound selectively binds to said polypeptide, wherein a determination that said compound selectively binds to said polypeptide indicates that said compound is a candidate activator of said polypeptide.

88. A method of identifying a candidate activator of a NF-HEV polypeptide of Paragraph 50 or Paragraph 52 or a fragment comprising a contiguous span of at least 6 contiguous amino acids of a polypeptide according to Paragraph 50 or Paragraph 52, said method comprising: a) contacting said polypeptide with a test compound; and b) determining whether said compound selectively increases at least one activity of said polypeptide, wherein a determination that said compound selectively increases at least one activity of said polypeptide indicates that said compound is a candidate inhibitor of said polypeptide.

89. A method of identifying a candidate NF-HEV activator, said method comprising: a) providing a cell comprising a NF-HEV polypeptide or a fragment comprising at least 6 consecutive amino acids thereof; b) contacting said cell with a test compound; and c) determining whether said compound selectively activates at least one NF-HEV biological activity, wherein a determination that said compound selectively activates the activity of said polypeptide indicates that said compound is a candidate activator of said polypeptide.

90. The method of Paragraph 87 wherein step (a) comprises introducing a nucleic acid comprising the nucleotide sequence encoding said NF-HEV polypeptide according to any one of Paragraphs 38, 39, 42 or 43 into said cell.

91. The method of Paragraphs 87 to 89, wherein said NF-HEV activity comprises modulating gene expression in an endothelial cell.

92. The method of Paragraphs 87 to 89, wherein said NF-HEV activity comprises modulating the inflammatory potential of an endothelial cell.

93. The method of Paragraphs 87 to 89, wherein said NF-HEV activity comprises modulating the phenotype of an endothelial cell.

94. The method of Paragraphs 87 to 89, wherein said NF-HEV activity comprises regulating HEV-like vessel development or maintenance.

95. The method of Paragraphs 87 to 89, wherein said NF-HEV activity comprises modulating the differentiation or proliferation of an endothelial cell.

96. The nucleic acid of Paragraph 42, wherein polypeptide identity is determined using an algorithm selected from the group consisting of XBLAST with the parameters score=50 and wordlength=3, Gapped BLAST with the default parameters of XBLAST, and BLAST with the default parameters of XBLAST.

97. The polypeptide of Paragraph 50, wherein identity is determined using an algorithm selected from the group consisting of XBLAST with the parameters score=50 and wordlength=3, Gapped BLAST with the default parameters of XBLAST, and BLAST with the default parameters of XBLAST.

98. An isolated nucleic acid encoding a biologically active NF-HEV polypeptide, said polypeptide comprising an amino acid sequence encoding the DNA-binding domain of the NF-HEV polypeptide, a biologically fragment thereof, or a polypeptide having at least 80% amino acid identity thereto.

99. An isolated nucleic acid encoding a biologically active NF-HEV polypeptide, said polypeptide comprising the amino acid sequence of amino acid positions 1 to 65 of SEQ ID NO: 4, a biologically fragment thereof, or a polypeptide having at least 80% amino acid identity thereto.

100. An isolated nucleic acid encoding a biologically active NF-HEV polypeptide, said polypeptide comprising the amino acid sequence of amino acid positions 1 to 67 of SEQ ID NO: 5, a biologically fragment thereof, or a polypeptide having at least 80% amino acid identity thereto.

101. An isolated nucleic acid encoding a biologically active NF-HEV polypeptide, said polypeptide comprising the amino acid sequence of amino acid positions 61 to 78 of SEQ ID NO: 4, a biologically fragment thereof, or a polypeptide having at least 80% amino acid identity thereto.

102. An isolated nucleic acid encoding a biologically active NF-HEV polypeptide, said polypeptide comprising the amino acid sequence of amino acid positions 63 to 80 of SEQ ID NO: 5, a biologically fragment thereof, or a polypeptide having at least 80% amino acid identity thereto.

103. An isolated nucleic acid encoding a biologically active NF-HEV polypeptide, said polypeptide comprising the amino acid sequence of amino acid positions 1 to 65 of SEQ ID NO: 1, a biologically fragment thereof, or a polypeptide having at least 80% amino acid identity thereto.

104. A polypeptide comprising a contiguous span of at least 6 amino acids of a sequence selected from the group consisting of SEQ ID NOs: 4-5.

105. The polypeptide of Paragraph 50, wherein said polypeptide comprises a homeodomain-like helix-turn-helix DNA-binding domain, or a fragment thereof.

106. The polypeptide of Paragraph 50, wherein said polypeptide comprises a contiguous span of at least 6 amino acids of amino acid positions 1 to 65 of SEQ ID NO: 4.

107. The polypeptide of Paragraph 50, wherein said polypeptide comprises a contiguous span of at least 6 amino acids of amino acid positions 1 to 67 of SEQ ID NO: 5.

108. The polypeptide of Paragraph 50, wherein said polypeptide comprises a contiguous span of at least 6 amino acids of amino acid positions 61 to 78 of SEQ ID NO: 5.

109. The polypeptide of Paragraph 50, wherein said polypeptide comprises a contiguous span of at least 6 amino acids of amino acid positions 63 to 80 of SEQ ID NO: 5.

110. A method of assessing the biological activity of a NF-HEV polypeptide comprising: (a) providing a NF-HEV polypeptide or a fragment thereof; and (b) assessing the ability of the NF-HEV polypeptide to induce differentiation of an endothelial cell.

111. A method of assessing the biological activity of a NF-HEV polypeptide comprising: (a) providing a NF-HEV polypeptide or a fragment thereof; and (b) assessing the ability of the NF-HEV polypeptide to modulate gene expression in an endothelial cell.

112. A method of assessing the biological activity of a NF-HEV polypeptide comprising: (a) providing a NF-HEV polypeptide or a fragment thereof; and (b) assessing the DNA binding activity of the NF-HEV polypeptide.

113. The method of Paragraphs 110, 111 or 112, wherein step (a) comprises introducing to a cell a recombinant vector comprising a nucleic acid encoding a NF-HEV polypeptide.

114. The method of Paragraphs 110, 111 or 112, wherein said NF-HEV activity comprises modulating gene expression in an endothelial cell.

115. The method of Paragraphs 110, 111 or 112, wherein said NF-HEV activity comprises modulating the inflammatory potential of an endothelial cell.

116. The method of Paragraphs 110, 111 or 112, wherein said NF-HEV activity comprises modulating the phenotype of an endothelial cell.

117. The method of Paragraphs 110, 111 or 112, wherein said NF-HEV activity comprises regulating HEV-like vessel development or maintenance.

118. The method of Paragraphs 110, 111 or 112, wherein said NF-HEV activity comprises modulating the differentiation or proliferation of an endothelial cell.

119. A method of obtaining a nucleic acid sequence which is recognized by a NF-HEV polypeptide comprising contacting a pool of random nucleic acids with said NF-HEV polypeptide or a portion thereof and isolating a complex comprising said NF-HEV polypeptide and at least one nucleic acid from said pool.

120. The method of Paragraph 119, wherein said pool of nucleic acids are labeled.

121. The method of Paragraph 119, wherein said complex is isolated by performing a gel shift analysis.

122. A method of identifying a nucleic acid sequence which is recognized by a NF-HEV polypeptide comprising: (a) incubating a NF-HEV polypeptide with a pool of labelled random nucleic acids; (b) isolating a complex between said NF-HEV polypeptide and at least one nucleic acid from said pool; (c) performing an amplification reaction to amplify the at least one nucleic acid present in said complex; (d) incubating said at least one amplified nucleic acid with said NF-HEV polypeptide; (e) isolating a complex between said at least one amplified nucleic acid and said NF-HEV polypeptide; (f) repeating steps (c), (d) and (e) a plurality of times; and (g) determining the sequence of said nucleic acid in said complex.

123. A method of identifying a compound which inhibits the ability of a NF-HEV polypeptide to bind to a nucleic acid comprising: (a) incubating a NF-HEV polypeptide or a fragment thereof which recognizes a binding site in a nucleic acid with a nucleic acid containing said binding site in the presence or absence of a test compound; and (b) determining whether the level of binding of said NF-HEV polypeptide to said nucleic acid in the presence of said test compound is less than the level of binding in the absence of said test compound.

124. A method of assessing NF-HEV activity in a biological sample, said method comprising the steps of: (a) contacting a nucleic acid molecule comprising a binding site for a NF-HEV polypeptide with a biological sample from a subject or a NF-HEV polypeptide isolated from a biological sample from a subject, the polypeptide comprising the amino acid sequences of one of SEQ ID NOs: 4-5; and (b) assessing the binding between said nucleic acid molecule and a NF-HEV polypeptide, wherein a detection of decreased binding compared to a reference NF-HEV nucleic acid binding level indicates that said sample comprises a deficiency in NF-HEV activity.

125. A method of identifying a candidate inhibitor of NF-HEV activity, said method comprising: (a) providing a NF-HEV polypeptide of SEQ ID NOs: 4-5 or, a fragment comprising a contiguous span of at least 6 contiguous amino acids of a polypeptide according to SEQ ID NOs: 4-5; (b) providing a NF-HEV target polypeptide or a fragment thereof; and (c) determining whether a test compound selectively inhibits the ability of said NF-HEV polypeptide to bind to said NF-HEV target polypeptide, wherein a determination that said test compound selectively inhibits the ability of said NF-HEV polypeptide to bind to said NF-HEV target polypeptide indicates that said compound is a candidate inhibitor of NF-HEV activity.

126. The method of any one of Paragraphs 1, 14, 23 or 33, wherein said NF-HEV polypeptide or biologically active fragment thereof comprises an amino acid sequence selected from the group consisting of amino acids 1-65 of SEQ ID NOs: 4-6.

### BRIEF DESCRIPTION OF THE DRAWINGS

FIG. 1 shows an amino acid sequence alignment of human NF-HEV (hNF-HEV) (SEQ ID NO: 4) with its mouse (mNF-HEV) (SEQ ID NO: 6) and canine (caDVS27) (SEQ ID NO: 5) orthologs. Conserved residues are boxed. Black boxes indicate identical residues, whereas shaded boxes show similar amino acids. Dashed lines represent gaps introduced to align sequences. Sequence alignment was performed with ClustalW and colored with Boxshade. Each of these programs can be obtained on the internet. The program ClustalW can be accessed by typing the following, “http://www2.ebi.ac” into the address bar of a web browser followed immediately by “.uk/clustalw”. The program Boxshade can be accessed by typing the following, “http://www.ch.embnet” into the address bar of a web browser followed immediately by “.org/software/BOX_form.html”. The bipartite NLS and the three helices of the homeodomain-like Helix-Turn-Helix (HTH) putative DNA-binding motif are indicated.

FIG. 2 depicts the genomic structure of the human and mouse NF-HEV genes. Open boxes indicate non-translated exon sequence and black boxes coding exon sequence. The two genes share a similar organization with seven exons. A major difference is the size of the first intron, which is >9 kb in the human gene but only ˜2 kb in its mouse ortholog.

FIG. 3A-C displays the results of in situ hybridization a riboprobe to NF-HEV mRNA in HEVs of human tonsil, Peyer's patch and mesenteric lymph node. Hybridization was performed on paraformaldehyde-fixed sections with an RNA probe complementary to NF-HEV mRNA (antisense), and hybridization signal (red) occurs in HEVs of the T-cell zone around lymphoid follicles in tonsil (A), Peyer's patch (B), and mesenteric lymph node (C). Higher magnification (600×, right panels) reveals that the signal is confined to HEVECs and scattered surrounding cells. Hybridization with a sense probe produced no signal (left panels).

FIG. 4A-B shows the results of virtual northern and western blot analyses demonstrating preferential expression of NF-HEV in HEVECs. Virtual northern blot analysis of NF-HEV expression in HEVECs, PMECs, HUVECs or placenta tissue (A). PCR-generated full-length cDNAs from the various types of ECs were electrophoresed on a 1% agarose gel, transferred to nylon filters, and hybridized under high-stringency conditions with a ³²P-labeled human NF-HEV cDNA probe. Western blot analysis of extracts of tonsillar stroma, HEVECs, PMECs or HUVECs with rabbit antibodies to NF-HEV (B). A single band of ˜30 kDa was detected in extracts of tonsillar stroma and HEVECs.

FIG. 5A-B shows nuclear localization of ectopically-expressed, epitope-tagged NF-HEV protein in primary HUVECs (A) or immortalized HeLa epithelial cells (B). HUVECs and HeLa cells were transfected with myc-tagged NF-HEV expression vector, stained by indirect immunofluorescence with antibodies to myc and then analyzed by confocal laser scanning microscopy. Original magnification: 1000×.

FIG. 6A-C depicts in situ expression of NF-HEV protein in the nucleus of tonsillar HEVECs. Cryosections of human tonsils (4 μm, acetone-fixed) were double-stained with HEV-specific rat mAb MECA-79 (A) or antibodies to NF-HEV peptides (B). Two-color overlays reveal that NF-HEV immunoreactivity is associated with MECA-79-positive HEVECs (C). Counterstaining with the nuclear dye DAPI showed a clear nuclear localization of NF-HEV in MECA-79-positive HEVECs (right panels). No nuclear staining was observed with preimmune rabbit serum (not shown). Original magnification: ×600.

FIG. 7 shows a model of the three-dimensional structure of the homeodomain-like HTH motif of human NF-HEV (aa 1-65), based on its threading-derived homology with the crystallographic structure of the homeodomain DBD from Drosophila transcription factor engrailed (PDB code: 1DU0). The α-helices have been numbered in order and color-coded in brown. The potential DNA recognition helix (α-helix 3) is marked by a red arrow. The turn of the HTH motif is coded in blue. Molecular modelling was performed as described in Example 9.

FIG. 8 displays the results of RT-PCR analysis of NF-HEV expression in human HEVEC, rheumatoid arthritis endothelial cells (ECs) and Crohn's disease ECs. RT-PCR was performed as described in Example 10. Colon Tumor ECs and HeLa samples were used as cell type controls. Amplification of G3PDH was used as controls a positive gene expression control. All PCR reactions were done at the same time and the identity of the PCR products was confirmed by restriction mapping or sequencing.

FIG. 9A-B shows specific expression of NF-HEV mRNA in endothelial cells from small blood vessels in Crohn's disease (A, ISH with antisense probe). No signal was detected when in situ hybridization was performed with a control probe (13, ISH with sense probe).

FIG. 10 shows specific expression of NF-HEV mRNA in endothelial cells from HEV-like small blood vessels in Rheumatoid arthritis (ISH with NF-HEV antisense probe, green dots). HEV-like vessels endothelial cells were labeled by immunohistochemistry (IHC, red, cell membrane) with anti-DARC antibody.

FIG. 11 shows regulation of NF-HEV mRNA expression by different pro-inflammatory cytokines. Endothelial cells were exposed to recombinant cytokines for 16 hours prior to RNA isolation. Presented are the copy numbers of NF-HEV mRNA determined in stimulated HUVECs, PMECs and HEVECs. Data for HUVECs and PMECs represent the mean values of two independent experiments, HEVEC data from one experiment. All Real-Time PCRs were performed in duplicate.

FIG. 12A-C reveals induction of chemokines MCP-1/CCL2, GROa/CXCL1 and IL-8/CXCL8 at the protein level in NF-HEV tranduced cells. Fold change inductions of MCP-1 (A), GROα (B) and IL-8 (C) protein detectable in 1 hour supernatants and cell lysates were determined by ELISA in two individual NF-HEV transduced cultures (HUVEC I+NF-HEV+NGFR or HUVEC II+NF-HEV+NGFR) compared to untransduced HUVEC (HUVEC I or HUVEC ID) or HUVEC transduced with control vector (HUVEC I+NGFR or HUVEC II+NGFR).

FIG. 13A-D shows induction of chemokines MCP-1/CCL2 and GROα/CXCL1 in NF-HEV tranduced cells (C II+NF-HEV+NGFR), as revealed by immunofluorescence staining of cells grown on chamber slides and analyzed by conventional fluorescence or confocal microscopy (C-D). Only low levels of chemolines were observed in cells transduced with the control retrovirus vector (HUVEC II+NGFR) (A-B).

### DETAILED DESCRIPTION

Aspects of the present invention are based on the characterization of the NF-HEV protein, a nuclear factor protein expressed in endothelial cells from chronically inflamed tissues, and particularly HEVECs in individuals suffering from chronic inflammation.

NF-HEV has been identified based on its expression in HEVs, specialized postcapillary venules found in lymphoid tissues and nonlymphoid tissues during chronic inflammatory diseases that support a high level of lymphocyte extravasation from the blood. Lymphocyte migration to secondary lymphoid tissue and chronical inflammatory lesions are directed by multistep interactions between the circulating cells and the specialized endothelium of high endothelial venules (HEVs) and HEV-like vessels. To identify novel HEV genes, freshly purified HEV endothelial cells (HEVECs) and nasal polyp-derived microvascular endothelial cells (PMECs) were compared using the PCR-based method of suppression subtractive hybridization (SSH). This approach resulted in the cloning of NF-HEV (nuclear factor from HEVs), the first nuclear factor preferentially expressed in HEVECs. Virtual northern and western blot analyses showed strong expression of NF-HEV in HEVECs, compared to human umbilical vein endothelial cells (HUVECs) and PMECs. In situ hybridization and immunohistochemistry revealed that NF-HEV mRNA and protein are expressed at high levels and rather selectively by HEVECs in human tonsils, Peyers's patches and lymph nodes. The NF-HEV protein was found to contain a bipartite nuclear localization signal, and was targeted to the nucleus when ectopically expressed in HUVECs and HeLa cells. Furthermore, endogenous NF-HEV was found in situ to be confined to the nucleus of tonsillar HEVECs. Threading and molecular modeling studies indicated that the amino-terminal part of NF-HEV (aa 1-60) corresponds to a novel homeodomain-like Helix-Turn-Helix (HTH) DNA-binding domain. Similar to the atypical homeodomain transcription factor Prox-1, which plays a critical role in the induction of the lymphatic endothelium phenotype, NF-HEV is likely a key nuclear factors that controls the specialized HEV phenotype.

Additional experiments to determine the role of NF-HEV in inflammation were also performed. Further in situ hybridization studies revealed that NF-HEV is expressed in endothelial cells from small blood vessels in Crohn's disease and rheumatoid arthritis. Real time PCR experiments showed that NF-HEV mMNA is induced by pro-inflammatory cytokines. Microarray and real time PCR analyses revealed NF-HEV supplied to HUVEC cells on a retroviral vector induced the expression of pro-inflammatory chemokines. The results of the microarray analysis was confirmed using a variety of supplemental techniques such as ELISA and immunofluorescence staining. These results indicate that NF-HEV plays a role in the modulation of inflammation.

The NF-HEV Protein

In some embodiments of the present invention, a NF-HEV family member comprises an amino acid sequence of at least about 15, 20, 30, 40, 50, 70, 100, 150, 200, 250 or 270 amino acid residues in length, of which amino acid sequence at least about 99%, 98%, 95%, 90%, 50-80%, preferably at least about 60-70%, more preferably at least about 65% of the amino acid residues are identical or similar to the amino acid sequences shown in SEQ ID NOs: 4, 5 or 6. NF-HEV proteins have an amino acid sequence sufficiently homologous to an amino acid sequence presented in SEQ ID NOs: 4, 5 or 6 or are encoded by a nucleotide sequence sufficiently homologous to a sequence presented in SEQ ID NOs: 1, 2 or 3. As used herein, the term “sufficiently homologous” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least about 30-40% identity, preferably at least about 40-50% identity, more preferably at least about 50-60%, and even more preferably at least about 60-70%, 70-80%, 80%, 90%, 95%, 97%, 98%, 99% or 99.8% identity across the amino acid sequences of the domains and contain at least one and preferably two structural domains or motifs, are defined herein as sufficiently homologous. Furthermore, amino acid or nucleotide sequences which share at least about 30%, preferably at least about 40%, more preferably at least about 60%, 70%, 80%, 90%, 95%, 97%, 98%, 99% or 99.8% identity and share a common functional activity are defined herein as sufficiently homologous.

NF-HEV Activity

As used interchangeably herein, an “NF-HEV activity”, “biological activity of NF-HEV” or “functional activity of NF-HEV”, refers to an activity exerted by a NF-HEV protein, polypeptide or nucleic acid molecule as determined in vivo, or in vitro, according to techniques described herein or any techniques known in the art for assaying the activity of similar molecules. In one embodiment, a NF-HEV activity is a direct activity, such as an association with a NF-HEV-target molecule. As used herein, a “target molecule” is a molecule with which a NF-HEV protein binds or interacts in nature, such that NF-HEV-mediated function is achieved. A NF-HEV target molecule can be a NF-HEV protein or polypeptide of the present invention or a non-NF-HEV molecule. For example, a NF-HEV target molecule can be a non-NF-HEV protein molecule such as a transcription factor, or may be a non-NF-HEV molecule such as a nucleic acid molecule, preferably a regulatory sequence (e.g. promoter). Alternatively, a NF-HEV activity is an indirect activity, such as an activity mediated by interaction of the NF-HEV protein with a NF-HEV target molecule such that the target molecule modulates a downstream cellular activity (e.g., interaction of a NF-HEV molecule with a NF-HEV target molecule can modulate the activity of that target molecule on an intracellular signaling pathway, preferably a pro-inflammatory signaling pathway). In a preferred embodiment, a NF-HEV activity is selected from the group consisting of. (a) modulating gene expression in an endothelial cell, preferably in a HEVEC cell or in a cell from a HEV-like vessel; (b) modulating the inflammatory potential of an endothelial cell; (c) regulating endothelial cell, preferably HEVEC phenotype; (d) regulating (e.g. inducing or inhibiting) HEV-like vessel development or maintenance; and (e) modulating (e.g. inducing or inhibiting) the differentiation and/or proliferation of endothelial cell, preferably HEVEC cells, or in cells from HEV-like vessels.

NF-HEV activity may be assessed either in vitro or in vivo depending on the assay type and format.

NF-HEV Nucleic Acids

The present invention relates to the use of the human (SEQ ID NO: 1) NF-HEV cDNAs as well as the murine NF-HEV coding sequence (SEQ ID NO: 2) and the canine NF-HEV coding sequence (SEQ ID NO: 3). The human NF-HEV cDNA, which is approximately 2628 nucleotides in length encodes a protein which is approximately 270 amino acid residues in length. The mouse NF-HEV coding sequence, approximately 2486 nucleotides in length, encodes a protein which is approximately 266 amino acid residues in length.

One aspect of the invention pertains to the use of purified or isolated nucleic acid molecules that encode NF-HEV proteins or biologically active portions thereof, as well as nucleic acid fragments thereof, in therapeutic methods, in diagnostic and drug screening assays. Fragments may be used for example as hybridization probes to identify NF-HEV-encoding nucleic acids (e.g., NF-HEV mRNA) and fragments for use as probes (e.g. for detection of NF-HEV nucleic acid molecules) or primers (e.g. for sequencing, genotyping, amplification or mutation of NF-HEV nucleic acid molecules). As used herein, the term “nucleic acids” and “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA. Throughout the present specification, the expression “nucleotide sequence” may be employed to designate indifferently a polynucleotide or a nucleic acid. More precisely, the expression “nucleotide sequence” encompasses the nucleic material itself and is thus not restricted to the sequence information (i.e. the succession of letters chosen among the four base letters) that biochemically characterizes a specific DNA or RNA molecule. Also, used interchangeably herein are terms “nucleic acids”, “oligonucleotides”, and “polynucleotides”.

An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated NF-HEV nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequences as given in SEQ ID NOs: 1, 2 or 3, or a portion thereof, can be isolated using standard molecular biology techniques and the sequence information provided herein. Using all or portion of the nucleic acid sequences in SEQ ID NOs: 1, 2 or 3 as a hybridization probe, NF-HEV nucleic acid molecules can be isolated using standard hybridization and cloning techniques (e.g., as described in Sambrook, I., Fritsh, E. F., and Maniatis, T. Molecular Cloning. A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of the sequences given in SEQ ID NOs: 1, 2 or 3 can be isolated by the polymerase chain reaction (PCR) using synthetic oligonucleotide primers designed based upon the same sequences.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NF-HEV nucleotide sequences can be prepared by standard synthetic techniques, e.g., using an automated DNA synthesizer.

In some embodiments, an isolated nucleic acid molecule for use in methods of the invention comprises, consists essentially of, or consists of a nucleotide sequences shown in SEQ ID NOs: 1, 2 or 3, or fragments thereof. These cDNAs comprise sequences encoding the human NF-HEV protein (i.e., “the coding region”, as well as 5′ untranslated sequences and 3′ untranslated sequences. Alternatively, the nucleic acid molecule can comprise, consist essentially of, or consist of only the coding region as given in SEQ ID NOs: 4, 5 or 6.

Also encompassed by the NF-HEV nucleic acids of the invention are nucleic acid molecules which are complementary to NF-HEV nucleic acids described herein. In some embodiments, a complementary nucleic acid is sufficiently complementary to the nucleotide sequence shown in SEQ ID NOs: 1, 2 or 3, such that it can hybridize to the nucleotide sequence shown in SEQ ID NOs: 1, 2 or 3, thereby forming a stable duplex.

The preferred purified, isolated, or recombinant NF-HEV nucleic acids encode a NF-HEV polypeptide comprising, consisting essentially of, or consisting of the amino acid sequences given in SEQ ID NOs: 4, 5 or 6, or fragments thereof. For example, the purified, isolated or recombinant nucleic acid may comprise a genomic DNA or fragment thereof which encode the polypeptides in SEQ ID NOs: 4, 5 or 6 or a fragment thereof. Preferred polynucleotides of the invention also include purified, isolated, or recombinant NF-HEV cDNAs consisting of, consisting essentially of, or comprising the sequences shown in SEQ ID NOs: 1, 2 or 3 or fragments thereof. Particularly preferred nucleic acids of the invention include isolated, purified, or recombinant fragments of NF-HEV nucleic acids comprising a contiguous span of at least 12, 15, 18, 20, 25, 30, 35, 40, 50, 60, 70, 80, 90, 100, 150, 200, 500, 1000 or 2000 nucleotides of the sequences in SEQ ID NOs: 1, 2 or 3 or the complements thereof.

Moreover, an NF-HEV nucleic acid molecule can comprise only a portion of the nucleic acid sequences in SEQ ID NOs: 1, 2 or 3, for example a fragment which can be used as a probe or primer or a fragment encoding a biologically active portion of a NF-HEV protein. The probe/primer typically comprises substantially purified oligonucleotide. The oligonucleotide typically comprises a region of nucleotide sequence that hybridizes under stringent conditions to at least about 12, preferably about 25, more preferably about 40, 50, more than 75 consecutive nucleotides of a sequence in SEQ ID NOs: 1, 2 or 3, or a sequence complementary thereto. In an exemplary embodiment, a nucleic acid molecule of the present invention comprises a nucleotide sequence which is at least about 400, 500, 1000, preferably at least about 1000-1250, more preferably at least about 1250-1500, more preferably at least about 1500-1750 in length and hybridizes under stringent hybridization conditions to a nucleic acid molecule in SEQ ID NOs: 1, 2 or 3.

A nucleic acid fragment encoding a “biologically active portion of a NF-HEV protein” can be prepared by isolating a portion of the nucleotide sequence in SEQ ID NOs: 1, 2 or 3 which encodes a polypeptide having a NF-HEV biological activity (the biological activities of the NF-HEV proteins described herein), expressing the encoded portion of the NF-HEV protein (e.g., by recombinant expression in vitro or in vivo) and assessing the activity of the encoded portion of the NF-HEV protein.

NF-HEV nucleic acid molecules that differ from the nucleotide sequence shown in SEQ ID NOs: 1, 2 or 3 due to degeneracy of the genetic code and thus encode the same NF-HEV proteins as those encoded by the nucleotide sequence shown in SEQ ID NOs: 1, 2 or 3 can also be used. In another embodiment, such an isolated nucleic acid molecule comprises a nucleotide sequence encoding a protein comprising an amino acid sequence shown in SEQ ID NOs: 4, 5 or 6 or a fragment thereof.

In addition to the NF-HEV nucleotide sequences shown in SEQ ID NOs: 1, 2 or 3, it will be appreciated by those skilled in the art that DNA sequence polymorphisms that lead to changes in the amino acid sequences of the NF-HEV proteins may exist within a population (e.g., the human population). Such genetic polymorphism in the NF-HEV genes may exist among individuals within a population due to natural allelic variation. As used herein, the terms “gene” and “recombinant gene” refer to nucleic acid molecules comprising an open reading frame encoding a NF-HEV protein, preferably a mammalian NF-HEV protein. Such natural allelic variations can typically result in 1-5% variance in the nucleotide sequence of a NF-HEV gene. Thus, also to be used according to the invention are any and all such nucleotide variations and resulting amino acid polymorphisms in NF-HEV genes that are the result of natural allelic variation and, most preferably, that do not alter the functional activity of a NF-HEV protein.

Also useful are nucleic acid molecules encoding other NF-HEV family members, and thus which have a nucleotide sequence which differs from the NF-HEV sequences of SEQ ID NOs: 1, 2 or 3. For example, a cDNA encoding a NF-HEV family member can be identified based on the nucleotide sequence of human NF-HEV. Moreover, nucleic acid molecules encoding NF-HEV proteins from different species, and thus which have a nucleotide sequence which differs from the NF-HEV sequences of SEQ ID NOs: 1, 2 or 3 are intended to be within the scope of the invention. For example, a mouse NF-HEV cDNA can be identified based on the nucleotide sequence of a human NF-HEV. Such NF-HEV family members may be identified by hybridization to a NF-HEV nucleic acid or fragment thereof, amplification with primers derived from a NF-HEV nucleic acid or fragment thereof, or bioinformatic comparison with a NF-HEV nucleic acid or fragment thereof or a NF-HEV polypeptide or fragment thereof.

Nucleic acid molecules corresponding to natural allelic variants and homologues of the NF-HEV cDNAs of the invention can be isolated based on their homology to the NF-HEV nucleic acids disclosed herein using the cDNAs disclosed herein, or a portion thereof, as a hybridization probe according to standard hybridization techniques under stringent hybridization conditions.

As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 70%, more preferably at least about 80%, even more preferably at least about 85%, 90%, 95% or 98% homologous to each other typically remain hybridized to each other. Stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6 sodium chloride/sodium citrate (SSC) at about 45° C., followed by one or more washes in 0.2 SSC, 0.1% SDS at 50-65° C.; Preferably, an isolated nucleic acid molecule of the invention that hybridizes under stringent conditions to the sequences in SEQ ID NOs: 1, 2 or 3 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the NF-HEV sequences that may exist in the population, the skilled artisan will further appreciate that changes can be introduced by mutation into the nucleotide sequences in SEQ ID NOs: 1, 2 or 3 thereby leading to changes in the amino acid sequence of the encoded NF-HEV proteins, without altering the functional ability of the NF-HEV proteins. For example, nucleotide substitutions leading to amino acid substitutions at “non-essential” amino acid residues can be made in the sequences in SEQ ID NOs: 1, 2 or 3. A “non-essential” amino acid residue is a residue that can be altered from the wild-type sequence of NF-HEV (e.g., the sequences of SEQ ID NOs: 4, 5 or 6) without altering the biological activity, whereas an “essential” amino acid residue is required for biological activity. For example, amino acid residues that are conserved among the NF-HEV proteins of the present invention, are predicted to be less unamenable to alteration.

Accordingly, nucleic acid molecules encoding NF-HEV proteins may contain changes in amino acid residues that are not essential for activity. Such NF-HEV proteins differ in amino acid sequence from sequences in SEQ ID NOs: 4, 5 or 6 yet retain biological activity. In one embodiment, the isolated nucleic acid molecule comprises a nucleotide sequence encoding a protein, wherein the protein comprises an amino acid sequence at least about 60% homologous to an amino acid sequences of SEQ ID NOs: 4, 5 or 6. Preferably, the protein encoded by the nucleic acid molecule is at least about 65-70% homologous to a sequence of SEQ ID NOs: 1, 2 or 3, more preferably sharing at least about 75-80% identity with a sequences in SEQ ID NOs: 1, 2 or 3, even more preferably sharing at least about 85%, 90%, 92%, 95%, 97%, 98%, 99% or 99.8% identity with a sequence of SEQ ID NOs: 1, 2 or 3.

An isolated nucleic acid molecule encoding a NF-HEV protein homologous to the proteins in SEQ ID NOs: 4, 5 or 6 can be created by introducing one or more nucleotide substitutions, additions or deletions into a nucleotide sequences in SEQ ID NOs: 1, 2 or 3 such that one or more amino acid substitutions, additions or deletions are introduced into the encoded protein. Mutations can be introduced into the sequences in SEQ ID NOs: 1, 2 or 3 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a NF-HEV protein is preferably replaced with another amino acid residue from the same side chain family. Alternatively, in another embodiment, mutations can be introduced randomly along all or part of a NF-HEV coding sequence, such as by saturation mutagenesis, and the resultant mutants can be screened for NF-HEV biological activity to identify mutants that retain activity. Following mutagenesis of a sequence given in SEQ ID NOs: 1, 2 or 3, the encoded protein can be expressed recombinantly and the activity of the protein can be determined.

In a preferred embodiment, a mutant NF-HEV protein encoded by a NF-HEV nucleic acid of the invention can be assayed for NF-HEV-activity in any suitable assay, examples of which are provided herein.

Primers and probes of the invention can be prepared by any suitable method, including, for example, cloning and restriction of appropriate sequences and direct chemical synthesis by a method such as the phosphodiester method of Narang et al. (1979), the phosphodiester method of [Brown et al. (1979)], the diethylphosphoramidite method of Beaucage et al. (1981) and the solid support method described in EP 0 707 592.

Detection probes are generally nucleic acid sequences or uncharged nucleic acid analogs such as, for example peptide nucleic acids which are disclosed in International Patent Application WO 92/20702, morpholino analogs which are described in U.S. Pat. Nos. 5,185,444; 5,034,506 and 5,142,047. The probe may have to be rendered “non-extendable” in that additional dNTPs cannot be added to the probe. In and of themselves analogs usually are non-extendable and nucleic acid probes can be rendered non-extendable by modifying the 3′ end of the probe such that the hydroxyl group is no longer capable of participating in elongation. For example, the 3′ end of the probe can be functionalized with the capture or detection label to thereby consume or otherwise block the hydroxyl group.

Any of the polynucleotides of the present invention can be labeled, if desired, by incorporating any label known in the art to be detectable by spectroscopic, photochemical, biochemical, immunochemical, or chemical means. For example, useful labels include radioactive substances (including, ³²P, ³⁵S, 3H, 1251), fluorescent dyes (including, 5-bromodesoxyuridin, fluorescein, acetylaminofluorene, digoxigenin) or biotin. Preferably, polynucleotides are labeled at their 3′ and 5′ ends. Examples of non-radioactive labeling of nucleic acid fragments are described in the French patent No. FR-7810975 or by Urdea et al. (1988) or Sanchez-Pescador et al. (1988). In addition, the probes according to the present invention may have structural characteristics such that they allow the signal amplification, such structural characteristics being, for example, branched DNA probes as those described by Urdea et al. in 1991 or in the European patent No. EP 0 225 807 (Chiron).

A label can also be used to capture the primer, so as to facilitate the immobilization of either the primer or a primer extension product, such as amplified DNA, on a solid support. A capture label is attached to the primers or probes and can be a specific binding member which forms a binding pair with the solid's phase reagent's specific binding member (e.g. biotin and streptavidin). Therefore depending upon the type of label carried by a polynucleotide or a probe, it may be employed to capture or to detect the target DNA. Further, it will be understood that the polynucleotides, primers or probes provided herein, may, themselves, serve as the capture label. For example, in the case where a solid phase reagent's binding member is a nucleic acid sequence, it may be selected such that it binds a complementary portion of a primer or probe to thereby immobilize the primer or probe to the solid phase. In cases where a polynucleotide probe itself serves as the binding member, those skilled in the art will recognize that the probe will contain a sequence or “tail” that is not complementary to the target. In the case where a polynucleotide primer itself serves as the capture label, at least a portion of the primer will be free to hybridize with a nucleic acid on a solid phase. DNA Labeling techniques are well known to the skilled technician.

The probes of the present invention are useful for a number of purposes. They can be notably used in Southern hybridization to genomic DNA. The probes can also be used to detect PCR amplification products. They may also be used to detect mismatches in the NF-HEV gene or mRNA using other techniques.

Any of the nucleic acids, polynucleotides, primers and probes of the present invention can be conveniently immobilized on a solid support. Solid supports are known to those skilled in the art and include the walls of wells of a reaction tray, test tubes, polystyrene beads, magnetic beads, nitrocellulose strips, membranes, microparticles such as latex particles, sheep (or other animal) red blood cells, duracytes and others. The solid support is not critical and can be selected by one skilled in the art. Thus, latex particles, microparticles, magnetic or non-magnetic beads, membranes, plastic tubes, walls of microtiter wells, glass or silicon chips, sheep (or other suitable animal's) red blood cells and duracytes are all suitable examples. Suitable methods for immobilizing nucleic acids on solid phases include ionic, hydrophobic, covalent interactions and the like. A solid support, as used herein, refers to any material which is insoluble, or can be made insoluble by a subsequent reaction. The solid support can be chosen for its intrinsic ability to attract and immobilize the capture reagent. Alternatively, the solid phase can retain an additional receptor which has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member which is immobilized upon (attached to) the solid support and which has the ability to immobilize the capture reagent through a specific binding reaction. The receptor molecule enables the indirect binding of the capture reagent to a solid support material before the performance of the assay or during the performance of the assay. The solid phase thus can be a plastic, derivatized plastic, magnetic or non-magnetic metal, glass or silicon surface of a test tube, microtiter well, sheet, bead, microparticle, chip, sheep (or other suitable animal's) red blood cells, duracytes and other configurations known to those of ordinary skill in the art. The nucleic acids, polynucleotides, primers and probes of the invention can be attached to or immobilized on a solid support individually or in groups of at least 2, 5, 8, 10, 12, 15, 20, or 25 distinct polynucleotides of the invention to a single solid support. In addition, polynucleotides other than those of the invention may be attached to the same solid support as one or more polynucleotides of the invention.

The invention also comprises methods for detecting or identifying an endothelial cell, a HEVEC cell or a cell from a HEV or HEV-like vessel, and methods for detecting or identifying a HEV-like vessel. More preferably, the invention also comprises methods for detecting or identifying an endothelial cell, a HEVEC cell or a cell from a HEV-like vessel which is involved in chronic inflammation.

These methods are useful for in research protocols where it is desirable to identify such cells or vessels as well as in diagnostic procedures as discussed herein (e.g. inflammatory conditions).

Detecting the presence of an NF-HEV nucleic acid comprising a nucleotide sequence selected from a group consisting of a sequences of SEQ ID NOs: 1, 2 or 3, a fragment or a variant thereof and a complementary sequence thereto in a sample, said method comprising the following steps of: (a) bringing into contact a nucleic acid probe or a plurality of nucleic acid probes which can hybridize with a nucleotide sequence included in a nucleic acid selected form the group consisting of a nucleotide sequences of SEQ ID NOs: 1, 2 or 3, a fragment or a variant thereof and a complementary sequence thereto and the sample to be assayed; and (b) detecting the hybrid complex formed between the probe and a nucleic acid in the sample. Preferably, detecting the presence of a hybrid formed indicates that the sample is derived from an endothelial cell, a HEVEC cell or a cell from a HEV-like vessel. Preferably, detecting the presence of a hybrid formed indicates that the sample derived from a cell involved in chronic inflammation.

The invention further concerns a kit for detecting the presence of an NF-HEV nucleic acid comprising a nucleotide sequence selected from a group consisting of a nucleotide sequences of SEQ ID NOs: 1, 2 or 3, a fragment or a variant thereof and a complementary sequence thereto in a sample, said kit comprising: (a) a nucleic acid probe or a plurality of nucleic acid probes which can hybridize with a nucleotide sequence included in a nucleic acid selected form the group consisting of the nucleotide sequences of SEQ ID NOs: 1, 2 or 3, a fragment or a variant thereof and a complementary sequence thereto; and (b) optionally, the reagents necessary for performing the hybridization reaction.

In a first preferred embodiment of this detection method and kit, said nucleic acid probe or the plurality of nucleic acid probes are labeled with a detectable molecule. In a second preferred embodiment of said method and kit, said nucleic acid probe or the plurality of nucleic acid probes has been immobilized on a substrate.

Any polynucleotide provided herein may be attached in overlapping areas or at random locations on a solid support. Alternatively the polynucleotides of the invention may be attached in an ordered array wherein each polynucleotide is attached to a distinct region of the solid support which does not overlap with the attachment site of any other polynucleotide. Preferably, such an ordered array of polynucleotides is designed to be “addressable” where the distinct locations are recorded and can be accessed as part of an assay procedure. Addressable polynucleotide arrays typically comprise a plurality of different oligonucleotide probes that are coupled to a surface of a substrate in different known locations. The knowledge of the precise location of each polynucleotides location makes these “addressable” arrays particularly useful in hybridization assays. Any addressable array technology known in the art can be employed with the polynucleotides of the invention. One particular embodiment of these polynucleotide arrays is known as the Genechips, and has been generally described in U.S. Pat. No. 5,143,854; PCT publications WO 90/15070 and 92/10092.

Probes based on the NF-HEV nucleotide sequences can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a NF-HEV protein, such as by measuring a level of a NF-HEV-encoding nucleic acid in a sample of cells from a subject e.g., detecting NF-HEV mRNA levels or determining whether a genomic NF-HEV gene has been mutated or deleted.

NF-HEV Polypeptides and Anti-NF-HEV Antibodies

The invention also relates to the use of isolated NF-HEV proteins, and biologically active portions thereof, as well as polypeptide fragments suitable for use as immunogens to raise anti-NF-HEV antibodies. In one embodiment, native NF-HEV proteins can be isolated from cells or tissue sources by an appropriate purification scheme using standard protein purification techniques. In another embodiment, NF-HEV proteins are produced by recombinant DNA techniques. Alternative to recombinant expression, a NF-HEV protein or polypeptide can be synthesized chemically using standard peptide synthesis techniques.

SEQ ID NOs: 4, 5 and 6 show the amino acid sequences human, mouse and canine NF-HEV polypeptides, respectively.

An “isolated” or “purified” protein or biologically active portion thereof is substantially free of cellular material or other contaminating proteins from the cell or tissue source from which the NF-HEV protein is derived, or substantially free from chemical precursors or other chemicals when chemically synthesized. The language “substantially free of cellular material” includes preparations of NF-HEV protein in which the protein is separated from cellular components of the cells from which it is isolated or recombinantly produced. In one embodiment, the language “substantially free of cellular material” includes preparations of NF-HEV protein having less than about 30% (by dry weight) of non-NF-HEV protein (also referred to herein as a “contaminating protein”), more preferably less than about 20% of non-NF-HEV protein, still more preferably less than about 10% of non-NF-HEV protein, and most preferably less than about 5% non-NF-HEV protein. When the NF-HEV protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of NF-HEV protein in which the protein is separated from chemical precursors or other chemicals which are involved in the synthesis of the protein. In one embodiment, the language “substantially free of chemical precursors or other chemicals” includes preparations of NF-HEV protein having less than about 30% (by dry weight) of chemical precursors or non-NF-HEV chemicals, more preferably less than about 20% chemical precursors or non-NF-HEV chemicals, still more preferably less than about 10% chemical precursors or non-NF-HEV chemicals, and most preferably less than about 5% chemical precursors or non-NF-HEV chemicals.

The term “polypeptide” refers to a polymer of amino acids without regard to the length of the polymer; thus, peptides, oligopeptides, and proteins are included within the definition of polypeptide. This term also does not specify or exclude post-expression modifications of polypeptides, for example, polypeptides which include the covalent attachment of glycosyl groups, acetyl groups, phosphate groups, lipid groups and the like are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring.

The term “recombinant polypeptide” is used herein to refer to polypeptides that have been artificially designed and which comprise at least two polypeptide sequences that are not found as contiguous polypeptide sequences in their initial natural environment, or to refer to polypeptides which have been expressed from a recombinant polynucleotide.

Biologically active portions of a NF-HEV protein include peptides comprising amino acid sequences sufficiently homologous to or derived from the amino acid sequence of the NF-HEV protein, e.g., an amino acid sequence shown in SEQ ID NOs: 4, 5 or 6, which include less amino acids than the full length NF-HEV proteins, and exhibit at least one activity of a NF-HEV protein. Typically, biologically active portions comprise a domain or motif with at least one activity of the NF-HEV proteins. A biologically active portion of a NF-HEV protein can be a polypeptide which is, for example at least 15, 25, 40, 50, 75, 100, 150, 200, 250 or 270 amino acids in length.

In a preferred embodiment, the NF-HEV protein comprises, consists essentially of, or consists of the amino acid sequence shown in SEQ ID NOs: 4, 5 or 6. The invention also concerns the polypeptide encoded by a nucleotide sequences selected from the group consisting of the sequences in SEQ ID NOs: 1, 2 or 3, a complementary sequence thereof or a fragment thereto. The present invention embodies isolated, purified, and recombinant fragments of one NF-HEV polypeptide comprising a contiguous span of at least 6 amino acids, preferably at least 8 to 10 amino acids, more preferably at least 12, 15, 20, 25, 30, 40, 50, 100, 150, 200, 250 or 270 amino acids of a sequence of SEQ ID NOs: 4, 5 or 6. In other preferred embodiments the contiguous stretch of amino acids comprises the site of a mutation or functional mutation, including a deletion, addition, swap or truncation of the amino acids in the NF-HEV protein sequence.

In other embodiments, the NF-HEV protein is substantially homologous to a sequence of SEQ ID NOs: 4, 5 or 6, and retains the functional activity of a protein of SEQ ID NOs: 4, 5 or 6, yet differs in amino acid sequence due to natural allelic variation or mutagenesis, as described in detail in subsection I above. Accordingly, in another embodiment, the NF-HEV proteins are proteins which comprise an amino acid sequence at least about 60% homologous to an amino acid sequence of SEQ ID NOs: 4, 5 or 6 and retain the functional activity of the NF-HEV proteins of SEQ ID NOs: 4, 5 or 6. Preferably, the proteins are at least about 30%, 40%, 50%, 60%, 70%, 80%, 85%, 90%, 92%, 95%, 97%, 98%, 99% or 99.8% homologous to a protein of SEQ ID NOs: 4, 5 or 6.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90% or 95% of the length of the reference sequence (e.g., when aligning a second sequence to a NF-HEV amino acid sequences of SEQ ID NO: 4 having 270 amino acid residues, at least 100, preferably at least 200, more preferably at least 250, even more preferably 270 amino acid residues are aligned or when aligning a second sequence to a NF-HEV nucleic acid sequence of SEQ ID NO: 1, preferably a human NF-HEV sequence comprising, consisting essentially of or consisting of 2628 nucleotides which encode the amino acids of the NF-HEV protein, preferably at least 100, preferably at least 200, more preferably at least 300, even more preferably at least 400, and even more preferably at least 500, 600, at least 700, at least 800, at least 900, or more than 1000 nucleotides are aligned. The amino acid residues or nucleotides at corresponding amino acid positions or nucleotide positions are then compared. When a position in the first sequence is occupied by the same: amino acid residue or nucleotide as the corresponding position in the second sequence, then the molecules are homologous at that position (i.e., as used herein amino acid or nucleic acid “identity” is equivalent to amino acid or nucleic acid “homology”). The percent homology between the two sequences is a function of the number of identical positions shared by the sequences (i.e., % homology=number (#) of identical positions/total number (#) of positions 100).

The comparison of sequences and determination of percent homology between two sequences can be accomplished using a mathematical algorithm. A preferred, non-limiting example of a mathematical algorithm utilized for the comparison of sequences is the algorithm of Karlin and Altschul (1990) Proc. Natl. Acad. Sci. USA 87:226-468, modified as in Karlin and Altschul (1993) Proc. Natl. Acad. Sci. USA 90:5873-77. Such an algorithm is incorporated into the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to NF-HEV nucleic acid molecules of the invention. BLAST protein searches can be performed with the XBLAST program, score=50, wordlength=3 to obtain amino acid sequences homologous to NF-HEV protein molecules of the invention. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

It is proposed that the monoclonal antibodies of the present invention will find useful application in standard immunochemical procedures, such as ELISA and Western blot methods and in immunohistochemical procedures such as tissue staining, as well as in other procedures which may utilize antibodies specific to NF-HEV antigen epitopes. Additionally, it is proposed that monoclonal antibodies specific to the particular NF-HEV of different species may be utilized in other useful applications In general, both polyclonal and monoclonal antibodies against NF-HEV may be used in a variety of embodiments. For example, they may be employed in antibody cloning protocols to obtain cDNAs or genes encoding other NF-HEV. They may also be used in inhibition studies to analyze the effects of NF-HEV-related peptides in cells or animals. NF-HEV antibodies will also be useful in immunolocalization studies to analyze the distribution of NF-HEV during various cellular events, for example, to determine the cellular or tissue-specific distribution of NF-HEV polypeptides at different points in the cell cycle. A particularly useful application of such antibodies is in purifying native or recombinant NF-HEV, for example, using an antibody affinity column. The operation of such immunological techniques will be known to those of skill in the art in light of the present disclosure.

The invention also provides NF-HEV chimeric or fusion proteins. As used herein, a NF-HEV “chimeric protein” or “fusion protein” comprises a NF-HEV polypeptide operatively linked, preferably fused in frame, to a non-NF-HEV polypeptide. In a preferred embodiment, a NF-HEV fusion protein comprises at least one biologically active portion of a NF-HEV protein. In another preferred embodiment, a NF-HEV fusion protein comprises at least two biologically active portions of a NF-HEV protein. For example, in one embodiment, the fusion protein is a OST-NF-HEV fusion protein in which the NF-HEV sequences are fused to the C-terminus of the GST sequences. Such fusion proteins can facilitate the purification of recombinant NF-HEV. In another embodiment, the fusion protein is a NF-HEV protein containing a heterologous signal sequence at its N-terminus, such as for example to allow for a desired cellular localization in a certain host cell. The NF-HEV-fusion proteins of the invention can be used for example as immunogens to produce anti-NF-HEV antibodies in a subject, to purify NF-HEV ligands and in screening assays to identify molecules which inhibit the interaction of NF-HEV with a NF-HEV target molecule.

The present invention also pertains to use of variants of the NF-HEV proteins which function as either NF-HEV mimetics or as NF-HEV inhibitors. Variants of the NF-HEV proteins can be generated by mutagenesis, e.g., discrete point mutation or truncation of a NF-HEV protein. An agonist of the NF-HEV proteins can retain substantially the same, or a subset, of the biological activities of the naturally occurring form of a NF-HEV protein. An antagonist of a NF-HEV protein can inhibit one or more of the activities of the naturally occurring form of the NF-HEV protein by, for example, competitively inhibiting the sulfate transport activity of a NF-HEV protein. Thus, specific biological effects can be elicited by treatment with a variant of limited function. In one embodiment, variants of a NF-HEV protein which function as either NF-HEV agonists (mimetics) or as NF-HEV antagonists can be identified by screening combinatorial libraries of mutants, e.g., truncation mutants, of a NF-HEV protein for NF-HEV protein agonist or antagonist activity. In one embodiment, a variegated library of NF-HEV variants is generated by combinatorial mutagenesis at the nucleic acid level and is encoded by a variegated gene library. A variegated library of NF-HEV variants can be produced by, for example, enzymatically ligating a mixture of synthetic oligonucleotides into gene sequences such that a degenerate set of potential NF-HEV sequences is expressible as individual polypeptides, or alternatively, as a set of larger fusion proteins (e.g., for phage display) containing the set of NF-HEV sequences therein. There are a variety of methods which can be used to produce libraries of potential NF-HEV variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NF-HEV sequences.

In addition, libraries of fragments of a NF-HEV protein coding sequence can be used to generate a variegated population of NF-HEV fragments for screening and subsequent selection of variants of a NF-HEV protein. In one embodiment, a library of coding sequence fragments can be generated by treating a double stranded PCR fragment of a NF-HEV coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the NF-HEV protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NF-HEV proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected.

An isolated NF-HEV protein, or a portion or fragment thereof, can be used as an immunogen to generate antibodies that bind NF-HEV using standard techniques for polyclonal and monoclonal antibody preparation. A full-length NF-HEV protein can be used or, alternatively, the invention provides antigenic peptide fragments of NF-HEV for use as immunogens. Any fragment of the NF-HEV protein which contains at least one antigenic determinant may be used to generate antibodies. The antigenic peptide of NF-HEV comprises at least 8 amino acid residues of the amino acid sequences shown in SEQ ID NOs: 4, 5 or 6 and encompasses an epitope of NF-HEV such that an antibody raised against the peptide forms a specific immune complex with NF-HEV. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of NF-HEV that are located on the surface of the protein, e.g., hydrophilic regions.

A NF-HEV immunogen typically is used to prepare antibodies by immunizing a suitable subject, (e.g., rabbit, goat, mouse or other mammal) with the immunogen. An appropriate immunogenic preparation can contain, for example, recombinantly expressed NF-HEV protein or a chemically synthesized NF-HEV polypeptide. The preparation can further include an adjuvant, such as Freund's complete or incomplete adjuvant, or similar immunostimulatory agent. Immunization of a suitable subject with an immunogenic NF-HEV preparation induces a polyclonal anti-NF-HEV antibody response.

Accordingly, another aspect of the invention pertains to anti-NF-HEV antibodies. The term “antibody” as used herein refers to immunoglobulin molecules and immunologically active portions of immunoglobulin molecules, i.e., molecules that contain an antigen binding site which specifically binds (immunoreacts with) an antigen, such as NF-HEV. Examples of immunologically active portions of immunoglobulin molecules include F(ab) and F(ab′)2 fragments which can be generated by treating the antibody with an enzyme such as pepsin. The invention provides polyclonal and monoclonal antibodies that bind NP-HEV. The term “monoclonal antibody” or “monoclonal antibody composition”, as used herein, refers to a population of antibody molecules that contain only one species of an antigen binding site capable of immunoreacting with a particular epitope of NF-HEV. A monoclonal antibody composition thus typically displays a single binding affinity for a particular NF-HEV protein with which it immunoreacts.

The invention concerns antibody compositions, either polygonal or monoclonal, capable of selectively binding, or selectively bind to an epitope-containing a polypeptide comprising a contiguous span of at least 6 amino acids, preferably at least 8 to 10 amino acids, more preferably at least 12, 15, 20, 25, 30, 40, 50, 100, or more than 100 amino acids in a sequence of SEQ ID NOs: 4, 5 or 6. The invention also concerns a purified or isolated antibody capable of specifically binding to a mutated NF-HEV proteins or to a fragment or variant thereof comprising an epitope of the mutated NF-HEV proteins.

Polyclonal anti-NF-HEV antibodies can be prepared as described above by immunizing a suitable subject with a NF-HEV immunogen. The anti-NF-HEV antibody titer in the immunized subject can be monitored over time by standard techniques, such as with an enzyme linked immunosorbent assay (ELISA) using immobilized NF-HEV. If desired, the antibody molecules directed against NF-HEV can be isolated from the mammal (e.g., from the blood) and further purified by well known techniques, such as protein A chromatography to obtain the IgG fraction. At an appropriate time after immunization, e.g., when the anti-NF-HEV antibody titers are highest, antibody-producing cells can be obtained from the subject and used to prepare monoclonal antibodies by standard techniques, such as those described in the following references: the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a NF-HEV immunogen as described above, and the culture supernatants of the resulting hybridoma cells are screened to identify a hybridoma producing a monoclonal antibody that binds NF-HEV.

Any of the many well known protocols used for fusing lymphocytes and immortalized cell lines can be applied for the purpose of generating an anti-NF-HEV monoclonal antibody (see, e.g., G. Galfre et al. (1977) Nature 266:55052; Gefter et al. Somatic Cell Genet., cited supra; Lerner, Yale J. Biol. Med, cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from American Type Culture Collection (ATCC). Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind NF-HEV, e.g., using a standard ELISA assay.

Alternative to preparing monoclonal antibody-secreting hybridomas, a monoclonal anti-NF-HEV antibody can be identified and isolated by screening a recombinant combinatorial immunoglobulin library (e.g., an antibody phage display library) with NF-HEV to thereby isolate immunoglobulin library members that bind NF-HEV. Kits for generating and screening phage display libraries are commercially available (e.g., the Pharmacia Recombinant Phage Antibody System, Catalog No. 27-9400-01; and the Stratagene SurfzAP™. Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc. Acid Res. 19:4133-4137; Barbas et al. (1991) PNAS 88:7978-7982; and McCafferty et al. Nature (1990) 348:552-554.

Additionally, recombinant anti-NF-HEV antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-NF-HEV antibody (e.g., monoclonal antibody) can be used to isolate NF-HEV by standard techniques, such as affinity chromatography or immunoprecipitation. An anti-NF-HEV antibody can facilitate the purification of natural NF-HEV from cells and of recombinantly produced NF-HEV expressed in host cells. Moreover, an anti-NF-HEV antibody can be used to detect NF-HEV protein (e.g., in a cellular lysate or cell supernatant) in order to evaluate the abundance and pattern of expression of the NF-HEV protein. Anti-NF-HEV antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125 I, 131 I, 35 S or 3H.

Recombinant Expression Vectors and Host Cells

Another aspect of the invention pertains to vectors, preferably expression vectors, containing a nucleic acid encoding a NF-HEV protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a NF-HEV nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NF-HEV proteins, mutant forms of NF-HEV proteins, fusion proteins, or fragments of any of the preceding proteins, etc.).

The recombinant expression vectors of the invention can be designed for expression of NF-HEV proteins in prokaryotic or eukaryotic cells. For example, NF-HEV proteins can be expressed in bacterial cells such as E. coli, insect cells (using baculovirus expression vectors) yeast cells, or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.), which fuse glutathione S-transferase (GST), maltose E binding protein, or protein A, respectively, to the target recombinant protein.

Purified fusion proteins can be utilized in NF-HEV activity assays, (e.g., direct assays or competitive assays described in detail below), or to generate antibodies specific for NF-HEV proteins, for example. In a preferred embodiment, a NF-HEV fusion protein expressed in a retroviral expression vector of the present invention can be utilized to infect bone marrow cells which are subsequently transplanted into irradiated recipients. The pathology of the subject recipient is then examined after sufficient time has passed (e.g. six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a co-expressed viral RNA polymerase (M7 gn 1). This viral polymerase is supplied by host strains BL21 (DE3) or HMS174(DE3) from a resident prophage harboring a T7 gnl gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the NF-HEV expression vector is a yeast expression vector. Examples of vectors for expression in yeast S. cerevisiae include pYepSec 1 (Baldari, et al., (1987) Embo J. 6:229-234), pMFa (Kuan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, NF-HEV proteins can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39). In particularly preferred embodiments, NF-HEV proteins are expressed according to Karniski et al, Am. J. Physiol. (1998) 275: F79-87.

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters bessel and Gruss (1990) Science 249:374-379) and the alpha-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to NF-HEV mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such term refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a NF-HEV protein can be expressed in bacterial cells such as E. coli, insect cells, yeast or mammalian cells (such as Chinese hamster ovary cells (CHO) or COS cells or human cells). Other suitable host cells are known to those skilled in the art, including Xenzopus laevis oocytes as further described in the Examples.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a NF-HEV protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a NF-HEV protein. Accordingly, the invention further provides methods for producing a NF-HEV protein using the host cells of the invention. In one embodiment, the method comprises culturing the host cell of invention (into which a recombinant expression vector encoding a NF-HEV protein has been introduced) in a suitable medium such that a NF-HEV protein is produced. In another embodiment, the method further comprises isolating a NF-HEV protein from the medium or the host cell.

In another embodiment, the invention encompasses providing a cell capable of expressing a NF-HEV protein, culturing said cell in a suitable medium such that a NF-HEV protein is produced, and isolating or purifying the NF-HEV protein from the medium or cell.

The host cells of the invention can also be used to produce nonhuman transgenic animals. Transgenic animals (for example an animal having a disrupted NF-HEV gene) may be useful for examining the development of HEV-like vessels. For example, in one embodiment, a host cell of the invention is a fertilized oocyte or an embryonic stem cell into which NF-HEV-coding sequences have been introduced. Such host cells can then be used to create non-human transgenic animals in which exogenous NF-HEV sequences have been introduced into their genome or homologous recombinant animals in which endogenous NF-HEV sequences have been altered. Such animals are useful for studying the function and/or activity of a NF-HEV polypeptide or fragment thereof and for identifying and/or evaluating modulators of NF-HEV activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NF-HEV gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a NF-HEV-encoding nucleic acid into the male pronuclei of a fertilized oocyte, e.g., by microinjection or retroviral infection, and allowing the oocyte to develop in a pseudopregnant female foster animal. The NF-HEV cDNA sequence or a fragment thereof such as a sequence of SEQ ID NO: 1 can be introduced as a transgene into the genome of a non-human animal. Alternatively, a nonhuman homologue of a human NF-HEV gene, such as a mouse or rat NF-HEV gene of SEQ ID NO: 2, can be used as a transgene. Intronic sequences and polyadenylation signals can also be included in the transgene to increase the efficiency of expression of the transgene. A tissue-specific regulatory sequence(s) can be operably linked to a NF-HEV transgene to direct expression of a NF-HEV protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a NF-HEV transgene in its genome and/or expression of NF-HEV mRNA in tissues or cells of the animals. A transgenic founder animal can then be used to breed additional animals carrying the transgene. Moreover, transgenic animals carrying a transgene encoding a NF-HEV protein can further be bred to other transgenic animals carrying other transgenes.

To create an animal in which a desired nucleic acid has been introduced into the genome via homologous recombination, a vector is prepared which contains at least a portion of a NF-HEV gene into which a deletion, addition or substitution has been introduced to thereby alter, e.g., functionally disrupt, the NF-HEV gene. The NF-HEV gene can be a human gene (e.g., the cDNA of SEQ ID NO: 1), but more preferably, is a non-human homologue of a human NF-HEV gene (e.g., a cDNA isolated by stringent hybridization with a nucleotide sequence of SEQ ID NO: 1). For example, a mouse NF-HEV gene of SEQ ID NO: 2 can be used to construct a homologous recombination vector suitable for altering an endogenous NF-HEV gene in the mouse genome. In a preferred embodiment, the vector is designed such that, upon homologous recombination, the endogenous NF-HEV gene is functionally disrupted (i.e., no longer encodes a functional protein; also referred to as a “knock out” vector). Alternatively, the vector can be designed such that, upon homologous recombination, the endogenous NF-HEV gene is mutated or otherwise altered but still encodes functional protein (e.g., the upstream regulatory region can be altered to thereby alter the expression of the endogenous NF-HEV protein). In the homologous recombination vector, the altered portion of the NF-HEV gene is flanked at its 5′ and 3′ ends by additional nucleic acid sequence of the NF-HEV gene to allow for homologous recombination to occur between the exogenous NF-HEV gene carried by the vector and an endogenous NF-HEV gene in an embryonic stem cell. The additional flanking NF-HEV nucleic acid sequence is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503, for a description of homologous recombination vectors). The vector is introduced into an embryonic stem cell line (e.g., by electroporation) and cells in which the introduced NF-HEV gene has homologously recombined with the endogenous NF-HEV gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells. A Practical Approach, E. J. Robertson, ed. (ERL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of Saccharomyces cerevisiae (O'Gorman et al. (1991) Science 251:1351-1355). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Drug Screening Assays

The invention provides a method (also referred to herein as a “screening assay”) for identifying inhibitors, i.e., candidate or test compounds or agents (e.g., preferably small molecules, but also peptides, peptidomimetics or other drugs) which bind to NF-HEV proteins, have an inhibitory or activating effect on, for example, NF-HEV expression or preferably NF-HEV activity, or have an inhibitory or activating effect on, for example, the activity of an NF-HEV target molecule. In some embodiments small molecules can be generated using combinatorial chemistry or can be obtained from a natural products library. Assays may be cell based or non-cell based assays. Drug screening assays may be binding assays or more preferentially functional assays, as further described.

Particularly preferred compounds will be those useful in inhibiting or promoting the actions of NF-HEV in regulating chronic inflammation, particularly in regulating the pro-inflammatory potential of an endothelial cell. Compounds may be useful in inhibiting or promoting the actions of NF-HEV in regulating the expression of proteins involved in inflammation. Compounds may also be useful in inhibiting or promoting the actions of NF-HEV in regulating the development and differentiation of endothelial cells or HEVECs. In the screening assays of the present invention, the candidate substance may first be screened for basic biochemical activity—e.g., binding to a target molecule—and then tested for its ability to modulate activity, at the cellular, tissue or whole animal level. The invention thus encompasses compounds capable of inhibiting or activating activity of the NF-HEV protein. Preferably, a NF-HEV inhibitor or activator is a selective NF-HEV inhibitor or activator.

In one aspect, a test compound may be identified based on binding to NF-HEV. One technique for high throughput screening of compounds is described in WO 84/03564. Large numbers of small peptide test compounds are synthesized on a solid substrate, such as plastic pins or some other surface. The peptide test compounds are reacted with, for example, NF-HEV and washed. Bound polypeptide is detected by various methods. Purified polypeptide, such as NF-HEV, can be coated directly onto plates for use in the aforementioned drug screening techniques. Alternatively, non-neutralizing antibodies to the polypeptide can be used to immobilize the polypeptide to a solid phase. Also, fusion proteins containing a reactive region (preferably a terminal region) may be used to link an active region (e.g., the C-terminus of NF-HEV) to a solid phase. Thus, in one embodiment, the present invention is directed to a method comprising: (a) providing a NF-HEV polypeptide; (b) contacting the NF-HEV polypeptide with a candidate substance; and (c) determining the binding of the candidate substance to NF-HEV polypeptide.

In preferred embodiments, an assay is a cell-based assay in which a cell which expresses a NF-HEV protein or biologically active portion thereof is contacted with a test compound and the ability of the test compound to inhibit, activate, or increase NF-HEV activity determined. Determining the ability of the test compound to inhibit, activate, or increase NF-HEV activity can be accomplished by monitoring the bioactivity of the NF-HEV protein or biologically active portion thereof. The cell, for example, can be of mammalian origin, bacterial origin or a yeast cell.

In one aspect, modulating inflammation comprises modulating transcription of genes involved in a pro-inflammatory pathway. In another aspect, modulating inflammation and/or modulating the endothelial cell or HEVEC phenotype comprises modulating transcription of genes involved in regulation (e.g. preferably involved in differentiation, proliferation or maintenance) of the endothelial cell, or preferably HEVEC, phenotype. Thus, in preferred aspects, the invention involves methods of screening that comprise measuring the effect of the candidate substance on the expression of an endothelial cell or HEVEC marker or any marker generally characterized as related to cells from HEV-like vessels. In one aspect, the invention comprises: (a) introducing to the cell an inhibitor of an NF-HEV polypeptide; (b) optionally, providing to the cell a NF-HEV polypeptide; (c) optionally, providing to the cell a polynucleotide encoding an additional polypeptide factor, preferably a transcription factor; and (d) detecting expression or transcription of an endothelial cell or HEVEC marker. More preferably, the screening method comprises: (a) introducing to the cell an inhibitor of an NF-HEV polypeptide; (b) optionally, introducing to a cell an expression cassette comprising a polynucleotide encoding a NF-HEV polypeptide operatively linked to a promoter capable of directing of expression of the polypeptide; (c) optionally, introducing to a cell an expression cassette comprising a polynucleotide encoding an additional polypeptide factor, preferably a transcription factor, said polynucleotide operatively linked to a promoter capable of directing of expression of the polypeptide; and (d) detecting expression or transcription of an endothelial cell or HEVEC marker.

In one aspect of the methods, the expression of an endothelial cell or HEVEC mRNA or polypeptide is detected. For example, staining by the HEV-specific MECA-79 sulfated epitope (Michie et al. (1993) Am J Pathol 143:1688-1698; Streeter et al. (1988) J Cell Biol 107:1853-1862) or the HECA452-fucosylated epitope (Duijvestijn et al. (1988) Am J Path 130:147-155) can be used to detect HEVECs. In another example, hybridization of a nucleic acid probe having a sequence complementary to the L-selectin ligand N-acetyl-glucosamine-6-O-sulfotransferase (LSST) (Bistrup et al. (1999) J Cell Biol 145:899-910; Hemmerich et al. (2001) Immunity 15:237-47; Hiraoka et al. (1999) Immunity 11:79-89), the fucosyltransferase FucTVII (Maly et al. (1996) Cell 86:643-653; Smith et al. (1996) J Biol Chem 271:8250-8259), the chemokine CCL21 (SLC/6Ckine/TCA4/exodus-2) (Gunn et al. (1998) Proc Natl Acad Sci USA 95:258-63) or the SPARC-like antiadhesive matricellular protein hevin (Girard and Springer (1995) Immunity 2:113-123; Girard and Springer (1996) J Biol Chem 271:4511-4517) mRNA can be detected. In another example, the method comprises introducing to the cell an expression cassette comprising a polynucleotide encoding a detectable polypeptide operatively linked to a transcriptional regulatory sequence of a gene encoding an endothelial cell or HEVEC marker. The effects of an inhibitor of NF-HEV on transcription of an endothelial cell or HEVEC marker can then be determined by assessing expression of the detectable polypeptide.

In one example, determining the ability of the test compound to inhibit or increase NF-HEV activity can be accomplished, by coupling the NF-HEV protein or biologically active portion thereof with a radioisotope or enzymatic label such that binding of the NF-HEV protein or biologically active portion thereof to its cognate target molecule can be determined by detecting the labelled NF-HEV protein or biologically active portion thereof in a complex. For example, compounds (e.g., NF-HEV protein or biologically active portion thereof) can be labelled with 125 I, 35 S, 14 C, or 3H, either directly or indirectly, and the radioisotope detected by direct counting of radioemmission or by scintillation counting. Alternatively, compounds can be enzymatically labelled with, for example, horseradish peroxidase, alkaline phosphatase, or luciferase, and the enzymatic label detected by determination of conversion of an appropriate substrate to product. The labelled molecule is placed in contact with its cognate molecule and the extent of complex formation is measured. For example, the extent of complex formation may be measured by immunoprecipitating the complex or by performing gel electrophoresis.

It is also within the scope of this invention to determine the ability of a compound (e.g., NF-HEV protein or biologically active portion thereof) to interact with its cognate target molecule without the labelling of any of the interactants. For example, a microphysiometer can be used to detect the interaction of a compound with its cognate target molecule without the labelling of either the compound or the target molecule. McConnell, H. M. et al. (1992) Science 257:1906-1912. A microphysiometer such as a cytosensor is an analytical instrument that measures the rate at which a cell acidifies its environment using a light-addressable potentiometric sensor (LAPS). Changes in this acidification rate can be used as an indicator of the interaction between compound and receptor.

In a preferred embodiment, the assay comprises contacting a cell which expresses a NF-HEV protein or biologically active portion thereof, with a target molecule to form an assay mixture, contacting the assay mixture with a test compound, and determining the ability of the test compound to inhibit or increase the activity of the NF-HEV protein or biologically active portion thereof, wherein determining the ability of the test compound to inhibit or increase the activity of the NF-HEV protein or biologically active portion thereof, comprises determining the ability of the test compound to inhibit or increase a biological activity of the NF-HEV expressing cell (e.g., determining the ability of the test compound to inhibit or increase transcription of a target nucleic acid, protein:protein interaction, nucleic acid binding).

In another preferred embodiment, the assay comprises contacting a cell which is responsive to a NF-HEV protein or biologically active portion thereof, with a NF-HEV protein or biologically-active portion thereof, to form an assay mixture, contacting the assay mixture with a test compound, and determining the ability of the test compound to modulate the activity of the NF-HEV protein or biologically active portion thereof, wherein determining the ability of the test compound to modulate the activity of the NF-HEV protein or biologically active portion thereof comprises determining the ability of the test compound to modulate a biological activity of the NF-HEV-responsive cell.

In another embodiment, an assay is a cell-based assay comprising contacting a cell expressing a NF-HEV target molecule (i.e. a molecule with which NF-HEV interacts) with a test compound and determining the ability of the test compound to modulate (e.g. stimulate or inhibit) the activity of the NF-HEV target molecule. Determining the ability of the test compound to modulate the activity of a NF-HEV target molecule can be accomplished, for example, by determining the ability of the NF-HEV protein to bind to or interact with the NF-HEV target molecule. An NF-HEV inhibitor may be capable of inhibiting or increasing the activity of or binding to more than one (e.g. at least two, three, four) nuclear factor proteins.

Determining the ability of the NF-HEV protein to bind to or interact with a NF-HEV target molecule can be accomplished by one of the methods described above for determining direct binding. In a preferred embodiment, determining the ability of the NF-HEV protein to bind to or interact with a NF-HEV target molecule can be accomplished by determining the activity of the target molecule. For example, the activity of the target molecule can be determined by contacting the target molecule with the NF-HEV protein or a fragment thereof and measuring induction of a cellular second messenger of the target (i.e. intracellular Ca2+, diacylglycerol, IP3, etc.), detecting catalytic/enzymatic activity of the target on an appropriate substrate, detecting the induction of a reporter gene (comprising a target-responsive regulatory element operatively linked to a nucleic acid encoding a detectable marker, e.g., luciferase), or detecting a target-regulated cellular response, for example, signal transduction or protein:protein interactions.

In yet another embodiment, an assay of the present invention is a cell-free assay in which a NF-HEV protein or biologically active portion thereof is contacted with a test compound and the ability of the test compound to bind to the NF-HEV protein or biologically active portion thereof is determined. Binding of the test compound to the NF-HEV protein can be determined either directly or indirectly as described above. In a preferred embodiment, the assay includes contacting the NF-HEV protein or biologically active portion thereof with a known compound which binds NF-HEV (e.g., a NF-HEV target molecule) to form an assay mixture, contacting the assay mixture with a test compound, and determining the ability of the test compound to interact with a NF-HEV protein, wherein determining the ability of the test compound to interact with a NF-HEV protein comprises determining the ability of the test compound to preferentially bind to NF-HEV or biologically active portion thereof as compared to the known compound.

In another embodiment, the assay is a cell-free assay in which a NF-HEV protein or biologically active portion thereof is contacted with a test compound and the ability of the test compound to modulate (e.g., stimulate or inhibit) the activity of the NF-HEV protein or biologically active portion thereof is determined. Determining the ability of the test compound to modulate the activity of a NF-HEV protein can be accomplished, for example, by determining the ability of the NF-HEV protein to bind to a NF-HEV target molecule by one of the methods described above for determining direct binding. Determining the ability of the NF-HEV protein to bind to a NF-HEV target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labelling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of a NF-HEV protein can be accomplished by determining the ability of the NF-HEV protein to further modulate the activity of a downstream effector (e.g., a component of a transcription regulation pathway) of a NF-HEV target molecule. For example, the activity of the effector molecule on an appropriate target can be determined or the binding of the effector to an appropriate target can be determined as previously described.

In yet another embodiment, the cell-free assay involves contacting a NF-HEV protein or biologically active portion thereof with a known compound which binds the NF-HEV protein to form an assay mixture, contacting the assay mixture with a test compound, and determining the ability of the test compound to interact with the NF-HEV protein, wherein determining the ability of the test compound to interact with the NF-HEV protein comprises determining the ability of the NF-HEV protein to preferentially bind to or modulate the activity of a NF-HEV target molecule.

The cell-free assays of the present invention are amenable to use of both soluble and/or membrane-bound forms of isolated proteins (e.g. NF-HEV proteins or biologically active portions thereof or molecules to which NF-HEV targets bind). In the case of cell-free assays in which a membrane-bound form an isolated protein is used it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton™ X-100, Triton™ X-114, Thesit™, Isotridecypoly(ethylene glycol ether)n,3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either NF-HEV or its target molecule to facilitate separation of complexed from uncomplexed forms of one or both of the proteins, as well as to accommodate automation of the assay. Binding of a test compound to a NF-HEV protein, or interaction of a NF-HEV protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/NF-HEV fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or NF-HEV protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of NF-HEV binding or activity determined using standard techniques.

Other techniques for immobilizing proteins on matrices can also be used in the screening assays of the invention. For example, either a NF-HEV protein or a NF-HEV target molecule can be immobilized utilizing conjugation of biotin and streptavidin. Biotinylated NF-HEV protein or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with NF-HEV protein or target molecules but which do not interfere with binding of the NF-HEV protein to its target molecule can be derivatized to the wells of the plate, and unbound target or NF-HEV protein trapped in the wells by antibody conjugation. Methods for detecting such complexes, in addition to those described above for the GST-immobilized complexes, include immunodetection of complexes using antibodies reactive with the NF-HEV protein or target molecule, as well as enzyme-linked assays which rely on detecting an enzymatic activity associated with the NF-HEV protein or target molecule.

In yet another aspect of the invention, the NF-HEV proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with NF-HEV (“NF-HEV-binding proteins” or “NF-HEV-bp”) and are involved in NF-HEV activity. Such NF-HEV-binding proteins are also likely to be involved in the propagation of signals by the NF-HEV proteins or NF-HEV targets as, for example, downstream elements of a NF-HEV-mediated signalling pathway or transcription system. Alternatively, such NF-HEV-binding proteins are likely to be NF-HEV inhibitors.

The two-hybrid system is based on the modular nature of most transcription factors, which consist of separable DNA-binding and activation domains. Briefly, the assay utilizes two different DNA constructs. In one construct, the gene that codes for a NF-HEV protein or a fragment thereof is fused to a gene encoding the DNA binding domain of a known transcription factor (e.g., GALA). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NF-HEV-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the NF-HEV protein.

In another embodiment, a NF-HEV target molecule is a nucleic acid (e.g. DNA). Assays of the invention are used to identify compounds that interfere with nucleic acid binding activity of NF-HEV, comprising the steps of: contacting a NF-HEV protein or a portion thereof comprising the DNA-binding domain immobilized on a solid support with both a test compound and polynucleotide fragments, or contacting a polynucleotide fragment immobilized on a solid support with both a test compound and a NF-HEV protein. The binding between the DNA and the NF-HEV-protein or portion thereof is detected, wherein a decrease in DNA binding when compared to polynucleotide binding in the absence of the test compound indicates that the test compound is an inhibitor of DNA binding activity, and an increase in DNA binding when compared to DNA binding in the absence of the test compound indicates that the test compound is an inducer of or restores NF-HEV DNA binding activity. As discussed further, DNA fragments may be selected to be specific NF-HEV protein target DNA obtained for example as described herein, or may be non-specific NF-HEV target DNA. Methods for detecting protein-DNA interactions are well known in the art, including most commonly used electrophoretic mobility shift assays (EMSAs) or by filter binding (Zabel et al, (1991) J. Biol. Chem., 266:252; and Okamoto and Beach, (1994) Embo J. 13: 4816). Other assays are available which are amenable for high tbroughput detection and quantification of specific and nonspecific DNA binding (Amersham, N.J.; and Gal S. et al, 6^{th} Ann. Conf. Soc. Biomol. Screening, 6-9 Sep. 2000, Vancouver, B.C.)

In a first aspect, a screening assay involves identifying compounds which interfere with NF-HEV DNA binding activity without prior knowledge about specific NF-HEV binding sequences. For example, a NF-HEV protein is contacted with both a test compound and a library of oligonucleotides or a sample of DNA fragments not selected based on specific DNA sequences. Preferably the NF-HEV protein is immobilized on a solid support (such as an array or a column). Unbound DNA is separated from DNA which is bound to the NF-HEV-family protein, and the DNA which is bound to NF-HEV protein is detected and can be quantitated by any means known in the art. For example, the DNA fragment is labeled with a detectable moiety, such as a radioactive moiety, a colorimetric moiety or a fluorescent moiety. Techniques for so labeling DNA are well known in the art.

The DNA which is bound to the NF-HEV protein or a portion thereof is separated from unbound DNA by immunoprecipitation with antibodies which are specific for the NF-HEV protein or a portion thereof. Use of two different monoclonal anti-NF-HEV antibodies may result in more complete immunoprecipitation than either one alone. The amount of DNA which is in the immunoprecipitate can be quantitated by any means known in the art. NF-HEV proteins or portions thereof which bind to the DNA can also be detected by gel shift assays (Tan, Cell, 62:367, 1990), nuclease protection assays, or methylase interference assays.

It is still another object of the invention to provide methods for identifying compounds which restore or the ability of mutant NF-HEV proteins or portions thereof or increase the ability of wild-type NF-HEV proteins to bind to DNA sequences. In one embodiment a method of screening agents for use in therapy is provided comprising: measuring the amount of binding of a NF-HEV protein or a portion thereof which is encoded by a mutant gene found in cells of a patient to DNA molecules, preferably random oligonucleotides or DNA fragments from a nucleic acid library; measuring the amount of binding of said NF-HEV protein or a portion thereof to said nucleic acid molecules in the presence of a test substance; and comparing the amount of binding of the NF-HEV protein or a portion thereof in the presence of said test substance to the amount of binding of the NF-HEV protein in the absence of said test substance, a test substance which increases the amount of binding being a candidate for use in therapy. In another embodiment of the invention, oligonucleotides can be isolated which restore or increase to NF-HEV proteins or portions thereof the ability to bind to a consensus binding sequence or conforming sequences. NF-HEV protein or a portion thereof and random oligonucleotides are added to a solid support on which NF-HEV-specific DNA fragments are immobilized. Oligonucleotides which bind to the solid support are recovered and analyzed. Those whose binding to the solid support is dependent on the presence of the NF-HEV protein are presumptively binding the support by binding to and restoring the conformation of the mutant protein.

If desired, specific binding can be distinguished from non-specific binding by any means known in the art. For example, specific binding interactions are stronger than non-specific binding interactions. Thus the incubation mixture can be subjected to any agent or condition which destabilizes protein/DNA interactions such that the specific binding reaction is the predominant one detected. Alternatively, as taught more specifically below, a non-specific competitor, such as dI-dC, can be added to the incubation mixture. If the DNA containing the specific binding sites is labeled and the competitor is unlabeled, then the specific binding reactions will be the ones predominantly detected upon measuring labeled DNA.

According to another embodiment of the invention, after incubation of NF-HEV protein or a portion thereof with specific DNA fragments all components of the cell lysate which do not bind to the DNA fragments are removed. This can be accomplished, among other ways, by employing DNA fragments which are attached to an insoluble polymeric support such as agarose, cellulose and the like. After binding, all non-binding components can be washed away, leaving NF-HEV protein or a portion thereof bound to the DNA/solid support. The NF-HEV protein or a portion thereof can be quantitated by any means known in the art. It can be determined using an immunological assay, such as an ELISA, RIA or Western blotting.

In another embodiment of the invention a method is provided for identifying compounds which specifically bind to NF-HEV-specific-DNA sequences, comprising the steps of: contacting a NF-HEV-specific DNA fragment immobilized on a solid support with both a test compound and wild-type NF-HEV protein or a portion thereof to bind the wild-type NF-HEV protein or a portion thereof to the DNA fragment; determining the amount of wild-type NF-HEV protein which is bound to the DNA fragment, inhibition of binding of wild-type NF-HEV protein by the test compound with respect to a control lacking the test compound suggesting binding of the test compound to the NF-HEV-specific DNA binding sequences.

It is still another object of the invention to provide methods for identifying compounds which restore the ability of mutant NF-HEV proteins or portions thereof to bind to specific DNA binding sequences. In one embodiment a method of screening agents for use in therapy is provided comprising: measuring the amount of binding of a NF-HEV protein or a portion thereof which is encoded by a mutant gene found in cells of a patient to a DNA molecule which comprises more than one monomer of a specific NF-HEV target nucleotide sequence; measuring the amount of binding of said NF-HEV protein to said nucleic acid molecule in the presence of a test substance; and comparing the amount of binding of the NF-HEV protein in the presence of said test substance to the amount of binding of the NF-HEV protein or a portion thereof in the absence of said test substance, a test substance which increases the amount of binding being a candidate for use in therapy.

In another embodiment of the invention a method is provided for screening agents for use in therapy comprising: contacting a transfected cell with a test substance, said transfected cell containing a NF-HEV protein or a portion thereof which is encoded by a mutant gene found in cells of a patient and a reporter gene construct comprising a reporter gene which encodes an assayable product and a sequence which conforms to a NF-HEV DNA binding site, wherein said sequence is upstream from and adjacent to said reporter gene; and determining whether the amount of expression of said reporter gene is altered by the test substance, a test substance which alters the amount of expression of said reporter gene being a candidate for use in therapy.

In still another embodiment a method of screening agents for use in therapy is provided comprising: adding RNA polymerase ribonucleotides and a NF-HEV protein or a portion thereof to a transcription construct, said transcription construct comprising a reporter gene which encodes an assayable product and a sequence which conforms to a NF-HEV consensus binding site, said sequence being upstream from and adjacent to said reporter gene, said step of adding being effected in the presence and absence of a test substance; determining whether the amount of transcription of said reporter gene is altered by the presence of said test substance, a test substance which alters the amount of transcription of said reporter gene being a candidate for use in therapy.

According to the present invention compounds which have NF-HEV activity are those which specifically complex with a NF-HEV-specific DNA binding site. Oligonucleotides and oligonucleotide containing nucleotide analogs are also contemplated among those compounds which are able to complex with a NF-HEV-specific DNA binding site.

In still yet other embodiments, one would look at the effect of a candidate substance on the expression of NF-HEV. This can be done by examining mRNA expression, although alterations in mRNA stability and translation would not be accounted for. A more direct way of assessing expression is by directly examining protein levels, for example, through Western blot or ELISA.

Preferably, modulators of NF-HEV expression are identified in a method wherein a cell is contacted with a candidate compound and the expression of NF-HEV mRNA or protein in the cell is determined. The level of expression of NF-HEV mRNA or protein in the presence of the candidate compound is compared to the level of expression of NF-HEV mRNA or protein in the absence of the candidate compound. The candidate compound can then be identified as a modulator of NF-HEV expression based on this comparison. For example, when expression of NF-HEV mRNA or protein is greater (statistically significantly greater) in the presence of the candidate compound than in its absence, the candidate compound is identified as a stimulator of NF-HEV mRNA or protein expression. Alternatively, when expression of NF-HEV mRNA or protein is less (statistically significantly less) in the presence of the candidate compound than in its absence, the candidate compound is identified as an inhibitor of NF-HEV mRNA or protein expression. The level of NF-HEV mRNA or protein expression can be determined by methods described herein for detecting NF-HEV mRNA or protein.

Another subject of the present invention is therefore a method for screening molecules that modulate the expression of the NF-HEV protein. Such a screening method comprises the steps of: (a) cultivating a prokaryotic or an eukaryotic cell that has been transfected with a nucleotide sequence encoding the NF-HEV protein or a variant or a fragment thereof, placed under the control of its own promoter; (b) bringing into contact the cultivated cell with a molecule to be tested; and (c) quantifying the expression of the NF-HEV protein or a variant or a fragment thereof.

Using DNA recombination techniques well known by the one skill in the art, the NF-HEV protein encoding DNA sequence is inserted into an expression vector, downstream from its promoter sequence. As an illustrative example, the promoter sequence of the NF-HEV gene is contained in the nucleic acid of the 5′ regulatory region.

The quantification of the expression of the NF-HEV protein may be realized either at the mRNA level or at the protein level. In the latter case, polyclonal or monoclonal antibodies may be used to quantify the amounts of the NF-HEV protein that have been produced, for example in an ELISA or a RIA assay. In a preferred embodiment, the quantification of the NF-HEV mRNA is realized by a quantitative PCR amplification of the cDNA obtained by a reverse transcription of the total mRNA of the cultivated NF-HEV-transfected host cell, using a pair of primers specific for NF-HEV.

The present invention also concerns a method for screening substances or molecules that are able to increase, or in contrast to decrease, the level of expression of the NF-HEV gene.

Thus, also part of the present invention is a method for screening of a candidate substance or molecule that modulated the expression of the NF-HEV gene, this method comprises the following steps: providing a recombinant cell host containing a nucleic acid, wherein said nucleic acid comprises a nucleotide sequence of the 5′ regulatory region or a biologically active fragment or variant thereof located upstream a polynucleotide encoding a detectable protein; obtaining a candidate substance; and determining the ability of the candidate substance to modulate the expression levels of the polynucleotide encoding the detectable protein.

In a further embodiment, the nucleic acid comprising the nucleotide sequence of the 5′ regulatory region or a biologically active fragment or variant thereof also includes a 5′UTR region of the NF-HEV cDNA, or one of its biologically active fragments or variants thereof.

Among the preferred polynucleotides encoding a detectable protein, there may be cited polynucleotides encoding beta galactosidase, green fluorescent protein (GFP) and chloramphenicol acetyl transferase (CAT).

The invention also pertains to kits useful for performing the herein described screening method. Preferably, such kits comprise a recombinant vector that allows the expression of a nucleotide sequence of the 5′ regulatory region or a biologically active fragment or variant thereof located upstream and operably linked to a polynucleotide encoding a detectable protein or the NF-HEV protein or a fragment or a variant thereof.

In another embodiment of a method for the screening of a candidate substance or molecule that modulates the expression of the NF-HEV gene, wherein said method comprises the following steps: (a) providing a recombinant host cell containing a nucleic acid, wherein said nucleic acid comprises a 5′UTR sequence of the NF-HEV cDNA, or one of its biologically active fragments or variants, the 5′UTR sequence or its biologically active fragment or variant being operably linked to a polynucleotide encoding a detectable protein; (b) obtaining a candidate substance; and (c) determining the ability of the candidate substance to modulate the expression levels of the polynucleotide encoding the detectable protein.

In a specific embodiment of the above screening method, the nucleic acid that comprises a nucleotide sequence selected from the group consisting of the 5′UTR sequence of the NF-HEV cDNA of or one of its biologically active fragments or variants, includes a promoter sequence which is endogenous with respect to the NF-HEV 5′UTR sequence. In another specific embodiment of the above screening method, the nucleic acid that comprises a nucleotide sequence selected from the group consisting of the 5′UTR sequence of the NF-HEV cDNA or one of its biologically active fragments or variants, includes a promoter sequence which is exogenous with respect to the NF-HEV 5′UTR sequence defined therein.

The invention further comprises with a kit for the screening of a candidate substance modulating the expression of the NF-HEV gene, wherein said kit comprises a recombinant vector that comprises a nucleic acid including a 5′UTR sequence of the NF-HEV cDNA, or one of their biologically active fragments or variants, the 5′UTR sequence or its biologically active fragment or variant being operably linked to a polynucleotide encoding a detectable protein.

For the design of suitable recombinant vectors useful for performing the screening methods described above, it will be referred to the section of the present specification wherein the preferred recombinant vectors of the invention are detailed.

Expression levels and patterns of NF-HEV may be analyzed by solution hybridization with long probes as described in International Patent Application No. WO 97/05277. Briefly, a NF-HEV cDNA or NF-HEV genomic DNA, or a fragment thereof, is inserted at a cloning site immediately downstream of a bacteriophage (T3, T7 or SP6) RNA polymerase promoter to produce antisense RNA. Preferably, the NF-HEV insert comprises at least 100 or more consecutive nucleotides of the genomic DNA sequence or the cDNA sequences. The plasmid is linearized and transcribed in the presence of ribonucleotides comprising modified ribonucleotides (i.e. biotin-UTP and DIG-UTP). An excess of this doubly labelled RNA is hybridized in solution with mRNA isolated from cells or tissues of interest. The hybridization is performed under standard stringent conditions (40-50° C. for 16 hours in an 80% formamide, 0.4 M NaCl buffer, pH 7-8). The unhybridized probe is removed by digestion with ribonucleases specific for single-stranded RNA (i.e. RNases CL3, T1, Phy M, U2 or A). The presence of the biotin-UTP modification enables capture of the hybrid on a microtitration plate coated with streptavidin. The presence of the DIG modification enables the hybrid to be detected and quantified by ELISA using an anti-DIG antibody coupled to alkaline phosphatase.

Quantitative analysis of NF-HEV gene expression may also be performed using arrays. As used herein, the term array means a one dimensional, two dimensional, or multidimensional arrangement of a plurality of nucleic acids of sufficient length to permit specific detection of expression of mRNAs capable of hybridizing thereto. For example, the arrays may contain a plurality of nucleic acids derived from genes whose expression levels are to be assessed. The arrays may include the NF-HEV genomic DNA, the NF-HEV cDNA sequences or the sequences complementary thereto or fragments thereof. In some embodiments, the fragments are at least 50 nucleotides in length. More preferably, the fragments are at least 100 nucleotides in length. In another preferred embodiment, the fragments are more than 100 nucleotides in length. In some embodiments the fragments may be more than 500 nucleotides in length.

For example, quantitative analysis of NF-HEV gene expression may be performed with a complementary DNA microarray as described by [Schena et al. (1995 and 1996)\. Full length NF-HEV cDNAs or fragments thereof are amplified by PCR and arrayed from a 96-well microtiter plate onto silylated microscope slides using high-speed robotics. Printed arrays are incubated in a humid chamber to allow rehydration of the array elements and rinsed, once in 0.2% SDS for 1 min, twice in water for 1 min and once for 5 min in sodium borohydride solution. The arrays are submerged in water for 2 min at 95° C., transferred into 0.2% SDS for 1 min, rinsed twice with water, air dried and stored in the dark at 25° C.

Cell or tissue mRNA is isolated or commercially obtained and probes are prepared by a single round of reverse transcription. Probes are hybridized to 1 cm² microarrays under a 14×14 mm glass coverslip for 6-12 hours at 60° C. Arrays are washed for 5 min at 25° C. in low stringency wash buffer (1×SSC/0.2% SDS), then for 10 min at room temperature in high stringency wash buffer (0.1×SSC/0.2% SDS). Arrays are scanned in 0.1×SSC using a fluorescence laser scanning device fitted with a custom filter set. Accurate differential expression measurements are obtained by taking the average of the ratios of two independent hybridizations.

Quantitative analysis of NF-HEV gene expression may also be performed with full length NF-HEV cDNAs or fragments thereof in complementary DNA arrays as described by Pietu et al. (1996). The full length NF-HEV cDNA or fragments thereof is PCR amplified and spotted on membranes. Then, mRNAs originating from various tissues or cells are labelled with radioactive nucleotides. After hybridization and washing in controlled conditions, the hybridized mRNAs are detected by phospho-imaging or autoradiography. Duplicate experiments are performed and a quantitative analysis of differentially expressed mRNAs is then performed.

Alternatively, expression analysis using the NF-HEV genomic DNA, the NF-HEV cDNA, or fragments thereof can be done through high density nucleotide arrays as described by Lockhart et al. (1996) and [Sosnowsky et al. (1997)]. Oligonucleotides of 15-50 nucleotides from the sequences of the NF-HEV DNA are synthesized directly on the chip (Lockhart et al., supra) or synthesized and then addressed to the chip (Sosnowski et al., supra). Preferably, the oligonucleotides are about 20 nucleotides in length.

NF-HEV cDNA probes labelled with an appropriate compound, such as biotin, digoxigenin or fluorescent dye, are synthesized from the appropriate mRNA population and then randomly fragmented to an average size of 50 to 100 nucleotides. The said probes are then hybridized to the chip. After washing as described in Lockhart et al., supra and application of different electric fields (Sosnowsky et al., 1997)., the dyes or labelling compounds are detected and quantified. Duplicate hybridizations are performed. Comparative analysis of the intensity of the signal originating from cDNA probes on the same target oligonucleotide in different cDNA samples indicates a differential expression of NF-HEV mRNA.

Test Compounds

This invention further pertains to novel agents identified by the above-described screening assays and to processes for producing such agents by use of these assays. Accordingly, in one embodiment, the present invention includes a compound or agent obtainable by a method comprising the steps of any one of the aforementioned screening assays (e.g., cell-based assays or cell-free assays). For example, in one embodiment, the invention includes a compound or agent obtainable by a method comprising contacting a cell which expresses a NF-HEV target molecule with a test compound and the determining the ability of the test compound to bind to, or modulate the activity of, the NF-HEV target molecule. In another embodiment, the invention includes a compound or agent obtainable by a method comprising contacting a cell which expresses a NF-HEV target molecule with a NF-HEV protein or biologically-active portion thereof, to form an assay mixture, contacting the assay mixture with a test compound, and determining the ability of the test compound to interact with, or modulate the activity of, the NF-HEV target molecule. In another embodiment, the invention includes a compound or agent obtainable by a method comprising contacting a NF-HEV protein or biologically active portion thereof with a test compound and determining the ability of the test compound to bind to, or modulate (e.g., stimulate or inhibit) the activity of, the NF-HEV protein or biologically active portion thereof. In yet another embodiment, the present invention included a compound or agent obtainable by a method comprising contacting a NF-HEV protein or biologically active portion thereof with a known compound which binds the NF-HEV protein to form an assay mixture, contacting the assay mixture with a test compound, and determining the ability of the test compound to interact with, or modulate the activity of the NF-HEV protein.

Accordingly, it is within the scope of this invention to further use an agent identified as described herein in an appropriate animal model. For example, an agent identified as described herein (e.g., a NF-HEV modulating agent, an antisense NF-HEV nucleic acid molecule, a NF-HEV-specific antibody, or a NF-HEV-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

The present invention also pertains to uses of novel agents identified by the above-described screening assays for diagnoses, prognoses, and treatments as described herein. Accordingly, it is within the scope of the present invention to use such agents in the design, formulation, synthesis, manufacture, and/or production of a drug or pharmaceutical composition for use in diagnosis, prognosis, or treatment, as described herein. For example, in one embodiment, the present invention includes a method of synthesizing or producing a drug or pharmaceutical composition by reference to the structure and/or properties of a compound obtainable by one of the above-described screening assays. For example, a drug or pharmaceutical composition can be synthesized based on the structure and/or properties of a compound obtained by a method in which a cell which expresses a NF-HEV target molecule is contacted with a test compound and the ability of the test compound to bind to, or modulate the activity of, the NF-HEV target molecule is determined. In another exemplary embodiment, the present invention includes a method of synthesizing or producing a drug or pharmaceutical composition based on the structure and/or properties of a compound obtainable by a method in which a NF-HEV protein or biologically active portion thereof is contacted with a test compound and the ability of the test compound to bind to, or modulate (e.g., stimulate or inhibit) the activity of, the NF-HEV protein or biologically active portion thereof is determined.

An inhibitor according to the present invention may be one which exerts an inhibitory effect on the expression or function of NF-HEV. By the same token, an activator according to the present invention may be one which exerts a stimulatory effect on the expression or function of NF-HEV. As used herein, the term “candidate substance”, “candidate compound” or “test compound” refers to any molecule that may potentially modulate NF-HEV expression or function. The candidate substance may be a protein or fragment thereof, a small molecule inhibitor, or even a nucleic acid molecule. It may prove to be the case that the most useful pharmacological compounds will be compounds that are structurally related to compounds which interact naturally with NF-HEV. Creating and examining the action of such molecules is known as “rational drug design,” and include making predictions relating to the structure of target molecules.

The goal of rational drug design is to produce structural analogs of biologically active polypeptides or target compounds. By creating such analogs, it is possible to fashion drugs which are more active or stable than the natural molecules, which have different susceptibility to, alteration or which may affect the function of various other molecules. In one approach, one would generate a three-dimensional structure for a molecule like NF-HEV, and then design a molecule for its ability to interact with NF-HEV. Alternatively, one could design a partially functional fragment of NF-HEV (binding, but no activity), thereby creating a competitive inhibitor. This could be accomplished by x-ray crystallography, computer modelling or by a combination of both approaches.

It also is possible to use antibodies to ascertain the structure of a target compound or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype. On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modelled of active, but otherwise undesirable compounds.

Candidate compounds may include fragments or parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or manmade compounds. Thus, it is understood that the candidate substance identified by the present invention may be polypeptide, polynucleotide, small molecule inhibitors or any other compounds that may be designed through rational drug design starting from known inhibitors of NF-HEV.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is used with peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew, Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devin (1990) Science 249:404-406); (Cwirla et al. (1990) Proc. Natl. Acad. Sci. 87:6378-6382); (Felici (1991) J. Mol. Biol. 222:301-310); (Ladner supra.).

Methods of Treatment

NF-HEV inhibitors identified according to the methods in the section titled “Drug Screening Assays” can be further tested for their ability to ameliorate or prevent inflammation, preferably chronic inflammation and autoimmune disorders in a suitable animal model of disease. Examples of animal models for rheumatoid arthritis include collagen-induced arthritis in mice (Gerlag et al. (2001) Arthritis Research 3:357-361; Bullard et al. (1996) J Immunol 157:3153-3158), adjuvant-induced arthritis in rats (Spargo et al., (1996) J Immunol 157:5198-5207), the rheumatoid arthritis transgenic mouse model (Kouskoff et al. (1996) Cell 87:811-822). Examples of animal models for Crohn's and inflammatory bowel diseases include several ‘gene knockout’ mice with inactivated IL2 (Sadlack et al. (1993) Cell 75:253-261), IL10 (Kun et al. (1993) Cell 75:263-274) or T cell receptor (Mombaerts et al. (1993) Cell 75:275-282; Mizoguchi et al. (1996) J Exp Med 184:707-715) genes, the T-cell mediated colitis model in SCID mice (Picarella et al. (1997) J Immunol 158:2099-2106), the cotton-top tamarin chronic colitis model (Podolsky et al. (1993) J Clin Invest 92: 272; Hesterberg et al. (1996) Gastroenterology 111:1373).

In one aspect, compounds capable of modulating NF-HEV may function by modulating the expression of a pro-inflammatory protein, particularly a protein involved in a pro-inflammatory signalling pathway. In another aspect, compounds capable of modulating NF-HEV may inhibit or prevent the development of HEV-like vessels. Since endothelial cells and particularly HEV-like vessels have several functions related to leukocyte adherence and extravasation, inflammation, and coagulation, compounds that interfere with HEV-like vessel development or maintenance can be used to modulate the pathological consequences of these events. Moreover, HEV-like vessels are known to develop at sites of inflammation resulting in further exacerbation of the inflammatory symptoms. Targeting HEV-like vessels for therapy has demonstrated that substantial decreases in lymphocyte migration can be achieved. For example functional inactivation of L-selectin by blocking antibodies (Gallatin et al, (1983) Nature 304: 30-34; Hamann et al, (1994) J. Immunol. 152: 3283-3293) or by gene knockout (Arbones et al, (1994) Immunity 1: 247-260) results in a 99% decrease of lymphocyte migration to peripheral lymph nodes (PLNs) and a 50% reduction of lymphocyte emigration in PP HEVs. The inhibitors therefore may be administered locally or systemically to control tissue damage associated with such injuries. Moreover, because of the specificity of such inhibitors for sites of inflammation, these compositions will be more effective and less likely to cause complications when compared to traditional anti-inflammatory agents.

NF-HEV inhibitors are expected to be particularly useful in the treatment of chronic inflammatory disorders. A review of disorders is provided in Girard and Springer, (1995) Immunology Today 16(9): 449-457. An inflammatory response can cause damage to the host if unchecked, because leukocytes release many toxic molecules that can damage normal tissues including proteolytic enzymes and free radicals. Vessels with HEV characteristics appear in human tissue in association with long-standing chronic inflammation. Such vessels exhibit plump endothelial cells, take up and incorporate high levels of ³⁵SO₄, contain many luminal and intramural lymphocytes (presumably in the process of extravasating) and mediate in vitro lymphocyte adhesion (Freemont (1998) J. Pathol. 155: 225-230).

The methods and compositions of the invention may be useful in the treatment of rheumatoid arthritis. Rheumatoid arthritis is characterized by symmetric, polyarticular inflammation of synovial-lined joints, and may involve extraarticular tissues, such as the pericardium, lung, and blood vessels. Adhesion molecules appear to play an important role (Postigo et al., Autoimmunity 16:69, 1993). Soluble selectins are present in the synovial fluid and blood of affected patients, correlating with elevated ESR and synovial PMN count (Carson C W et al. J. Rheumatol. 21:605, 1994). Conventional antirheumatic therapy may modify synovial inflammation by altering leukocyte adhesion. Corticosteroids, gold compounds, and colchicine down-regulate endothelial expression of selectins (Corkill et al., J. Rheumatol. 18:1453, 1991; Molad et al., Arthritis Rheum. 35:S35, 1992).

In rheumatoid arthritis, it has been observed that the level of sulfate incorporation as well as the ‘plumpness’ (or ‘tallness’) of the endothelium in areas of lymphocyte infiltration in the synovial membrane are closely related to the concentration of the lymphocytes in the perivascular infiltrates (Freemont, (1987) Ann. Rheum. Dis. 46: 924-928). Similarly, expression of MECA-79 and HECA-452 on these vessels is most pronounced in association with extensive lymphoid infiltrates (Michie et al, (1993) Am. J. Pathol. 143: 1688-1698; van Dinther-Janssen et al, (1990) J. Rheumatol. 17:11-17). Therefore, the development of bona fide HEVs in the synovial membrane of patients with rheumatoid arthritis is likely to facilitate large-scale influx of lymphocytes, leading to amplification and maintenance of chronic inflammation. Inhibition of HEVEC differentiation and HEV-like vessel development would therefore be useful for the treatment of rheumatoid arthritis.

The development of HEV-like vessels after prolonged inflammatory stimulus is not restricted to the diseased synovium, but can also occur in other tissues, particularly the gut and thyroid. During chronic inflammation of the gut in inflammatory bowel diseases (Crohn's disease and ulcerative colitis), or the thyroid in autoimmune thyroiditis (Graves' disease and Hashimoto's thyroiditis), areas of dense lymophocytic infiltration contain HEV-like vessels with plump endothelium expressing MECA-79 and HECA-452 (Michie et al, supra; Duijvestijn et al., (1988) Am. J. Pathol. 130: 147-155; Kabel et al., J. (1989) Clin. Endocrinol. Metab. 68: 744-751; and Salmi et al. (1994) Gastroenterology 106: 595-605). These observations suggest that HEV-lke vessels could play an important role in the pathogenesis of these diseases by mediating abnormal lymphocyte recruitment to the gut or the thyroid. MECA-79+ HEV-like venules with plump endothelium have also been detected in other sites of chronic inflammation, including many cutaneous inflammatory lesions (Michie et al, supra).

NF-HEV inhibitors may also be useful in the treatment of disorders characterized by extralymphoid sites of chronic inflammation. In one example, NF-HEV inhibitors may be useful for the treatment or prevention of diabetes mellitus. In the nonobese diabetic (NOD) mouse model of human insulin-dependent diabetes mellitus (IDDM), vessels with HEV characteristics (e.g. plump endothelial cells, numerous lymphocytes in the vessel walls) are observed during inflammation of the pancreas. Expression of MECA-79 and MECA-367 (MAdCAM-1) in induced on these HEV-like vessels (Hanninen et al., (1993) J. Clin. Invest. 92: 2509-2515; Faveeuw et al., (1994) J. Immunol. 152: 5969-5978) during the development of insulitis, whereby lymphocytes infiltrate the pancreatic islets. Staining with MECA-79 in consistent with the induction of functional L-selectin ligands, CD34, MAdCAM-1 and GlyCAM-1 (Baumhueter et al, (1994) Blood 84: 2554-2565). The induction of GlyCAM-1 in the inflamed pancreas of NOD mice is particularly striking since GlyCAM-1 expression in mice had previously been shown to be restricted to PLN and mesenteric lymph node (MLN) HEVs (Lasky et al., (1992) Cell 69: 927-938). Together these results indicate that HEV-like vessels induced by chronic inflammation in extralymphoid sites appear to be phenotypically similar to HEVs from lymphoid tissues. The induction of MECA-79 and MAdCAM-1 on the endothelium correlates with the expression of their counter-receptors L-selectin and alpha4-beta7 on cells infiltrating the islets (Hanninen et al., supra). In vivo studies have revealed that these two receptor-counter receptor pairs, alpha4-beta7-MAdCAM-1 and L-selectin-MECA-79, play a major role in the recruitment of lymphocytes from blood into the inflamed pancreas (Yang et al., (1993) PNAS USA 90: 10494-10498). Treatment of NOD mice with function-blocking monoclonal antibodies specific for L-selectin and alpha-4 integrins resulted in the inhibition of insulitis and the prevention of autoimmune diabetes.

In other examples, a NF-HEV inhibitor may be used for the treatment or prevention of graft rejection. L-selectin dependent lymphocyte extravasation as occurs through HEVs is a hallmark of acute heart allograft rejection in rates. Evidence further demonstrates a complete correlation between the level of expression of the sulfated sialyl Lewis-x decorated L-selectin ligands and the histological severity of heart allograft rejection (Toppila et al., (1999) Am. J. Pathol. 155:1013-1020), suggesting that NF-HEV inhibitors capable of blocking sulfation of L-selectin ligands may be capable of preventing lymphocyte extravasation into human heart allografts at the onset and during acute rejection episodes. In particular, Toppila et al showed that non-rejecting heart endothelium did not express, or expressed only weakly, sulfated and or sialyl Lewis-x decorations of L-selectin ligands, while said epitopes were readily detectable on endothelium of capillaries and venules at the onset and during acute rejection episodes. Molecules capable or preventing or reducing the formation of HEV-like vessels would thus reduce the sites available for lymphocyte extravasation.

Thus, the invention includes in preferred embodiments methods of inhibiting inflammation or more preferably chronic inflammation, as well as methods of modulating the expression of a pro-inflammatory protein, particularly a protein involved in a pro-inflammatory signalling pathway, methods of inhibiting leukocyte adhesion or migration, and yet more particularly methods of inhibiting development of HEV-like vessels or inhibiting differentiation of endothelial or HEVEC cells, the methods comprising administering a NF-HEV inhibitor.

Activators of NF-HEV activity may be used to treat conditions in which it is desired to obtain increased development of HEVECs or HEV-like vessels, particularly where it is desired to obtain increased lymphocyte infiltration (Schrama et al. (2001) Immunity 14:111-121). For example, NF-HEV activators may be used to enhance the infiltration of lymphocytes into solid tumors, such as melanoma and colon or breast carcinoma.

An “individual” treated by the methods of this invention is a vertebrate, particularly a mammal (including model animals of human disease), and typically a human.

As used herein, “treatment” refers to clinical intervention in an attempt to alter the natural course of the individual being treated, and may be performed either for prophylaxis or during the course of clinical pathology. Desirable effects include preventing occurrence or recurrence of disease, alleviation of symptoms, diminishment of any direct or indirect pathological consequences of the disease, such as hyperresponsiveness, inflammation, or necrosis, lowering the rate of disease progression, amelioration or palliation of the disease state, and remission or improved prognosis. The “pathology” associated with a disease condition is anything that compromises the well-being, normal physiology, or quality of life of the affected individual.

Treatment is performed by administering an effective amount of a NF-HEV inhibitor or activator. An “effective amount” is an amount sufficient to effect a beneficial or desired clinical result, and can be administered in one or more doses.

The criteria for assessing response to therapeutic modalities employing the lipid compositions of this invention are dictated by the specific condition, measured according to standard medical procedures appropriate for the condition.

Pharmaceutical Compositions

Compounds capable of inhibiting NF-HEV activity, preferably small molecules but also including peptides, NF-HEV nucleic acid molecules, NF-HEV proteins, and anti-NF-HEV antibodies (also referred to herein as “active compounds”) of the invention can be incorporated into pharmaceutical compositions suitable for administration. Such compositions typically comprise a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Where the active compound is a protein, peptide or anti-NF-HEV antibody, sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. For administration by inhalation, the compounds are delivered in the form of an aerosol spray from pressured container or dispenser which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Most preferably, active compound is delivered to a subject by intravenous injection.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or preferably parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

Diagnostics and Identification of HEVECs and HEV-Like Vessels

The nucleic acid molecules, proteins, protein homologues, and antibodies described herein can be of particular benefit in the identification of endothelial cells, HEVEC and HEV-like vessels involved in inflammation, preferably chronic inflammation. The compositions will also be useful in diagnostic assays, prognostic assays, monitoring clinical trials, and pharmacogenetics related to inflammatory disorders as further described herein.

According to the present invention, a NF-HEV protein, a NF-HEV-specific antibody, or a NF-HEV nucleic acid is used to distinguish endothelial cells, HEVECs or HEV-like vessels involved in inflammation from endothelial cells, or vessels that are not involved in inflammation or have decreased inflammatory potential. This is particularly useful in research and development, where there is a need for means that are capable of distinguishing endothelial cells from inflamed samples from other endothelial cells. In other aspects, the levels of NF-HEV expression in HEVEC cells indicates that NF-HEV can also be used to distinguish HEVECs from non-HEVEC cells.

The invention also involves methods of use (e.g., a diagnostic assay, prognostic assay, or a prophylactic/therapeutic method of treatment) wherein a NF-HEV protein, NF-HEV nucleic acid, or most preferably a NF-HEV inhibitor or activator, is used, for example, to diagnose, prognose and/or treat an inflammatory disorder, most preferably a chronic inflammatory disorder. In another embodiment, the methods of use (e.g., diagnostic assays, prognostic assays, or prophylactic/therapeutic methods of treatment) involve administering to a human subject a NF-HEV inhibitor or activator.

For example, the invention encompasses a method of determining whether NF-HEV is expressed within a biological sample comprising: a) contacting said biological sample with: ii) a polynucleotide that hybridizes under stringent conditions to a NF-HEV nucleic acid; or iii) a detectable polypeptide that selectively binds to a NF-HEV polypeptide; and b) detecting the presence or absence of hybridization between said polynucleotide and an RNA species within said sample, or the presence or absence of binding of said detectable polypeptide to a polypeptide within said sample. A detection of said hybridization or of said binding indicates that said NF-HEV is expressed within said sample and that the sample comprises nucleic acids or protein derived from an inflamed tissue, or more preferably from an endothelial cell involved in inflammation or having inflammatory potential. Preferably, the polynucleotide is a primer, and wherein said hybridization is detected by detecting the presence of an amplification product comprising said primer sequence, or the detectable polypeptide is an antibody.

Also envisioned is a method of determining whether a cell expresses a NF-HEV nucleic acid or polypeptide, comprising: a) providing a biological sample (e.g. sample of cells or sample from a mammal); and b) preferably comparing the amount of a NF-HEV polypeptide or of a NF-HEV RNA species encoding a NF-HEV polypeptide within said biological sample with a level detected in or expected from a control sample. Expression of said NF-HEV polypeptide or said NF-HEV RNA species within said biological sample indicates that the sample comprises nucleic acids or protein derived from an inflamed tissue, or more preferably from an endothelial cell involved in inflammation or having inflammatory potential. Also encompassed is a method of determining whether a cell or mammal, preferably human, has an elevated or reduced level of NF-HEV expression, comprising: a) providing a biological sample (e.g. sample of cells or sample from said mammal; and b) comparing the amount of a NF-HEV polypeptide or of a NF-HEV RNA species encoding a NF-HEV polypeptide within said biological sample with a level detected in or expected from a control sample. An increased amount of said NF-HEV polypeptide or said NF-HEV RNA species within said biological sample compared to said level detected in or expected from said control sample indicates that said mammal has an elevated level of NF-HEV expression, and wherein a decreased amount of said NF-HEV polypeptide or said NF-HEV RNA species within said biological sample compared to said level detected in or expected from said control sample indicates that said mammal has a reduced level of NF-HEV expression. As discussed, NF-HEV expression may be useful for identifying a HEVEC or HEV-like vessel involved in inflammation or having inflammatory potential as well as for identifying subjects suffering from or susceptible to suffering from chronic inflammatory conditions.

An exemplary method for detecting the presence or absence of NF-HEV protein or nucleic acid in a biological sample involves obtaining a biological sample from a test subject, for example by conducting a biopsy at a site of inflammation or suspected inflammation, and contacting the biological sample with a compound or an agent capable of detecting NF-HEV protein or nucleic acid (e.g., mRNA, genomic DNA) that encodes NF-HEV protein such that the presence of NF-HEV protein or nucleic acid is detected in the biological sample. A preferred agent for detecting NF-HEV mRNA or genomic DNA is a labelled nucleic acid probe capable of hybridizing to NF-HEV mRNA or genomic DNA. The nucleic acid probe can be, for example, a full-length NF-HEV nucleic acid, such as a nucleic acid of sequences of SEQ ID NOs: 1, 2 or 3 such as a nucleic acid of at least 15, 30, 50, 100, 250, 400, 500 or 1000 nucleotides in length and sufficient to specifically hybridize under stringent conditions to NF-HEV mRNA or genomic DNA. Other suitable probes for use in the diagnostic assays of the invention are described herein.

A preferred agent for detecting NF-HEV protein is an antibody capable of binding to NF-HEV protein, preferably an antibody with a detectable label. Antibodies can be polyclonal, or more preferably, monoclonal. An intact antibody, or a fragment thereof (e.g., Fab or F(ab′)2) can be used. The term “labelled”, with regard to the probe or antibody, is intended to encompass direct labelling of the probe or antibody by coupling (i.e., physically linking) a detectable substance to the probe or antibody, as well as indirect labelling of the probe or antibody by reactivity with another reagent that is directly labelled. Examples of indirect labelling include detection of a primary antibody using a fluorescently labelled secondary antibody and end-labelling of a DNA probe with biotin such that it can be detected with fluorescently labelled streptavidin. The term “biological sample” is intended to include tissues, cells and biological fluids isolated from a subject, as well as tissues, cells and fluids present within a subject. That is, the detection method of the invention can be used to detect NF-HEV mRNA, protein, or genomic DNA in a biological sample in vitro as well as in vivo. For example, in vitro techniques for detection of NF-HEV mRNA include Northern hybridizations and in situ hybridizations. In vitro techniques for detection of NF-HEV protein include enzyme linked immunosorbent assays (ELISAs), Western blots, immunoprecipitations and immunofluorescence. In vitro techniques for detection of NF-HEV genomic DNA include Southern hybridizations. Furthermore, in vivo techniques for detection of NF-HEV protein include introducing into a subject a labelled anti-NF-HEV antibody. For example, the antibody can be labelled with a radioactive marker whose presence and location in a subject can be detected by standard imaging techniques.

In one embodiment, the biological sample contains protein molecules from the test subject or test composition (e.g. composition of cells). Alternatively, the biological sample can contain mRNA molecules from the test subject or genomic DNA molecules from the test subject or test composition. In another embodiment, the methods further involve obtaining a control biological sample from a control subject, contacting the control sample with a compound or agent capable of detecting NF-HEV protein, mRNA, or genomic DNA, such that the presence of NF-HEV protein, mRNA or genomic DNA is detected in the biological sample, and comparing the presence of NF-HEV protein, mRNA or genomic DNA in the control sample with the presence of NF-HEV protein, mRNA or genomic DNA in the test sample. The invention also encompasses kits for detecting the presence of NF-HEV in a biological sample. For example, the kit can comprise a labelled compound or agent capable of detecting NF-HEV protein or mRNA in a biological sample; means for determining the amount of NF-HEV in the sample; and means for comparing the amount of NF-HEV in the sample with a standard. The compound or agent can be packaged in a suitable container. The kit can further comprise instructions for using the kit to detect NF-HEV protein or nucleic acid.

In certain embodiments, detection involves the use of a probe/primer in a polymerase chain reaction CPCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the NF-HEV-gene (see Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to a NF-HEV gene under conditions such that hybridization and amplification of the NF-HEV-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Genotyping assays for diagnostics can also be carried out. Genotyping assays may be useful, for example, to detect alleles associated with inflammatory disorders. Genotyping assays generally require the previous amplification of the DNA region carrying the allele of interest. However, ultrasensitive detection methods which do not require amplification are also available. Methods well-known to those skilled in the art that can be used to detect polymorphisms include methods such as, conventional dot blot analyzes, single strand conformational polymorphism analysis (SSCP) described by Orita et al. PNAS 86 : 2766-2770 (1989), denaturing gradient gel electrophoresis (DGGE), heteroduplex analysis, mismatch cleavage detection, and other conventional techniques as described in Sheffield et al. (1991), White et al. (1992), and Grompe et al. (1989 and 1993) (Sheffield, V. C. et al, Proc. Natl. Acad. Sci. U.S.A 49:699-706 (1991); White, M. B. et al., Genomics 12:301-306 (1992); Grompe, M. et al., Proc. Natl. Acad. Sci. U.S.A 86:5855-5892 (1989); and Grompe, M. Nature Genetics 5:111-117 (1993)). Another method for determining the identity of the nucleotide present at a particular polymorphic site employs a specialized exonuclease-resistant nucleotide derivative as described in U.S. Pat. No. 4,656,127. Further methods are described as follows. Other methods include microsequencing methods, in which the nucleotide at a polymorphic site in a target DNA is detected by a single nucleotide primer extension reaction. A homogeneous phase microsequencing-based detection method based on fluorescence resonance energy transfer has been described by Chen and Kwok (1997) and Chen et al. (1997) Chen and Kwok (Nucleic Acids Research 25:347-353 1997) and Chen et al. (Proc. Natl. Acad. Sci. USA 94/20 10756-10761, 1997)).

Modulation of NF-HEV Polypeptide Level and Activity

Some embodiments of the present invention relate to the modulation of the level or activity of the NF-HEV polypeptide or a biologically active fragment thereof using cytokines or other compounds involved in mediation of an inflammatory response. For example, in some embodiments, pro-inflammatory cytokines can be used to modulate the level of transcription from the NF-HEV gene. In other embodiments, pro-inflammatory cytokines are used to modulate the activity of NF-HEV protein. Pro-inflammatory cytokines include, but are not limited to, TNFα, IL1β and IFNγ.

Other embodiments of the present invention relate modulation of the level or activity of the NF-HEV polypeptide or a biologically active fragment thereof using molecules that inhibit, counteract or have a function contrary to the activity of a pro-inflammatory cytokine. Inhibitors of pro-inflammatory cytokines and methods of inhibiting such molecules are known in the art and have been described in U.S. Pat. Nos. 6,541,482; 6,420,373; 6,440,968.

Other embodiments of the present invention relate to methods of modulating the level or activity of the NF-HEV polypeptide or a biologically active fragment thereof by using molecules that do not modulate the level or activity of proinflammatory cytokines. In some embodiments, such molecules decrease the level or activity of the NF-HEV polypeptide or a biologically active fragment thereof. In some embodiments, these molecules can act directly on the NF-HEV gene and/or polypeptide in order to lower the expression level of the NF-HEV transcript or to reduce the activity of the NF-HEV polypeptide. Examples of molecules having the ability to inhibit the production of NF-HEV polypeptide include, but are not limited to, antisense nucleic acids and small inhibitory RNA (siRNAs).

Some embodiments of the present invention provide a method of producing sequence-specific inhibition of the expression of a gene and/or other nucleic acid which encodes the NF-HEV polypeptide or a biologically active fragment thereof using antisense nucleic acids. As such, some embodiments of the present invention relate to antisense nucleic acids that are used to reduce the amount of the NF-HEV polypeptide or a biologically active fragment thereof that is present inside a cell. In some embodiments, such antisense nucleic acids are complementary to at least a portion of the coding strand of SEQ ID NO: 1. Such antisense nucleic acids include antisense polynucleotides complementary to the full-length sense strand of a gene and/or other nucleic acid which encodes the NF-HEV polypeptide or a biologically active fragment thereof, or complementary to oligonucleotide fragments from at least about 15 to more than about 120 nucleotides, including at least about 16 nucleotides, at least about 17 nucleotides, at least about 18 nucleotides, at least about 19 nucleotides, at least about 20 nucleotides, at least about 21 nucleotides, at least about 22 nucleotides, at least about 23 nucleotides, at least about 24 nucleotides, at least about 25 nucleotides, at least about 26 nucleotides, at least about 27 nucleotides, at least about 28 nucleotides, at least about 29 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides, at least about 110 nucleotides, at least about 120 nucleotides or greater than 120 nucleotides.

As used herein, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

While antisense oligonucleotides are a preferred form of antisense compound, embodiments of the present invention contemplates other oligomeric antisense compounds, including but not limited to, oligonucleotide mimetics such as are described below. The antisense oligonucleotides described herein also include ribozymes, external guide sequence (EGS) oligonucleotides (oligozymes), and other short catalytic RNAs or catalytic oligonucleotides which hybridize to the target nucleic acid and modulate its expression.

Specific examples of antisense compounds useful in certain embodiments of this invention include oligonucleotides containing modified backbones or non-natural internucleoside linkages. As used herein, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

In some embodiments modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Certain oligonucleotides having inverted polarity comprise a single 3′ to 3′ linkage at the 3′-most internucleotide linkage, i.e. a single inverted nucleoside residue which can be abasic (the nucleobase is missing or has a hydroxyl group in place thereof). Various salts, mixed salts and free acid forms are also included.

In some embodiments, modified oligonucleotide backbones that do not include a phosphorus atom therein have backbones that are formed by short chain allyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other embodiments, oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262. Further teaching of PNA compounds can be found in Nielsen et al., Science, 1991, 254, 1497-1500.

In still other embodiments of the present invention, the expression of gene and/or other nucleic acid which encodes the NF-HEV polypeptide or a biologically active fragment thereof is modulated using oligonucleotides with phosphorothioate backbones and oligonucleosides with heteroatom backbones. Modified oligonucleotides may also contain one or more substituted sugar moieties. In some embodiments oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂ and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Another modification includes 2′-methoxyethoxy (2′ OCH₂CH₂OCH₃, also known as 2′-O-(2-methoxyethyl) or 2′-MOE) (Martin et al., Helv. Chim. Acta, 1995, 78, 486-504).

An embodiment of the present invention includes the use of Locked Nucleic Acids (LNAs) to generate antisense nucleic acids having enhanced affinity and specificity for the target polynucleotide. LNAs are nucleic acid in which the 2′-hydroxyl group is linked to the 3′ or 4′ carbon atom of the sugar ring thereby forming a bicyclic sugar moiety. The linkage is preferably a methelyne (—CH₂—)ₙ group bridging the 2′ oxygen atom and the 4′ carbon atom wherein n is 1 or 2. LNAs and preparation thereof are described in WO 98/39352 and WO 99/14226.

Other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine, 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrimido[3′,2′:4,5]pyrrolo[2,3-d]pyrimidin-2-one). Modified nucleobases may also include those in which the purine or pyrimidine base is replaced with other heterocycles, for example 7-deazaadenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. ed., CRC Press, 1993. Certain of these nucleobases are particularly useful for increasing the binding affinity of the oligomeric compounds described herein. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Another modification of the antisense oligonucleotides described herein involves chemically linking to the oligonucleotide one or more moieties or conjugates which enhance the activity, cellular distribution or cellular uptake of the oligonucleotide. The antisense oligonucleotides can include conjugate groups covalently bound to functional groups such as primary or secondary hydroxyl groups. Conjugate groups include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-s-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., dihexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylaminocarbonyloxycholesterol moiety (Crooke et al., J. Pharmacol. Exp. Ther., 1996, 277, 923-937.

It is not necessary for all positions in a given compound to be uniformly modified, and in fact more than one of the aforementioned modifications may be incorporated in a single compound or even at a single nucleoside within an oligonucleotide. The methods described herein also contemplate the use of antisense compounds which are chimeric compounds. “Chimeric” antisense compounds or “chimeras,” as used herein, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds for use in the methods of the present invention may be formed as composite structures of two or more oligonucleotides, modified oligonucleotides, oligonucleosides and/or oligonucleotide mimetics as described above. Such compounds have also been referred to in the art as hybrids or gapmers.

The antisense compounds used in accordance with some embodiments of this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The antisense compounds for use with the methods described herein encompass any pharmaceutically acceptable salts, esters, or salts of such esters, or any other compound which, upon administration to an animal including a human, is capable of providing (directly or indirectly) the biologically active metabolite or residue thereof.

In some embodiments of the present invention, an antisense nucleic acid specific to the gene and/or other nucleic acid which encodes the NF-HEV polypeptide or a biologically active fragment thereof is synthesized and introduced directly into a subject. In other embodiments, the antisense nucleic acid can be formulated as part of a targeted delivery system, such as a target specific liposome, which specifically recognizes and delivers the antisense nucleic acid to an appropriate tissue or cell type, such as an inflamed tissue or a HEVEC. Upon administration of the targeted antisense nucleic acid to a subject, the antisense nucleic acid is delivered to the appropriate cell type thereby increasing the concentration antisense nucleic acid within the cell type.

In other embodiments of the present invention, an appropriate cell or tissue is provided with expression construct which comprises a nucleic acid that encodes the antisense RNA that is specific to the gene and/or other nucleic acid which encodes the NF-HEV polypeptide or a biologically active fragment thereof. In these embodiments, the nucleic acid that encoding the antisense RNA can be placed under the control of either a constitutive or a regulatable promoter.

Some embodiments of the present invention provide a method of producing sequence-specific inhibition of the expression of a gene and/or other nucleic acid which encodes the NF-HEV polypeptide or a biologically active fragment thereof using siRNAs. As used herein siRNAs are synonymous with double-stranded RNA (dsRNA), and include double-stranded RNA oligomers with or without hairpin structures at each end. Small interfering RNAs comprise oligonucleotides of at least about 15 to greater than about 120 nucleotides, including at least about 16 nucleotides, at least about 17 nucleotides, at least about 18 nucleotides, at least about 19 nucleotides, at least about 20 nucleotides, at least about 21 nucleotides, at least about 22 nucleotides, at least about 23 nucleotides, at least about 24 nucleotides, at least about 25 nucleotides, at least about 26 nucleotides, at least about 27 nucleotides, at least about 28 nucleotides, at least about 29 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides, at least about 110 nucleotides, at least about 120 nucleotides or greater than 120 nucleotides. In certain embodiments of the present invention, the siRNA comprises an oligonucleotide from about 21 to about 25 nucleotides in length. In some embodiments, the siRNA molecule is a heteroduplex of RNA and DNA.

As with antisense nucleic acids, siRNAs can include modifications to either the phosphate-sugar backbone or the nucleoside. For example, the phosphodiester linkages of natural RNA may be modified to include at least one of a nitrogen or sulfur heteroatom. Modifications in RNA structure may be tailored as described for antisense nucleic acids.

A process for inhibiting expression of a gene and/or other nucleic acid which encodes the NF-HEV polypeptide or a biologically active fragment thereof in a cell comprises introduction of an siRNA with partial or fully double-stranded character into a cell. Inhibition is sequence-specific in that a nucleotide sequence from a portion of the gene and/or other nucleic acid which encodes the NF-HEV polypeptide or a biologically active fragment thereof is chosen to produce inhibitory RNA. Depending on the dose of siRNA delivered, this process can provide partial or complete loss of function for the gene and/or other nucleic acid which encodes the NF-REV polypeptide or a biologically active fragment thereof.

In some embodiments of the present invention, an siRNA specific to the gene and/or other nucleic acid which encodes the NF-HEV polypeptide or a biologically active fragment thereof is synthesized and introduced directly into a subject. In other embodiments, the siRNA can be formulated as part of a targeted delivery system, such as a target specific liposome, which specifically recognizes and delivers the siRNA to an appropriate tissue or cell type, such as an inflamed tissue or a HEVEC. Upon administration of the targeted siRNA to a subject, the siRNA is delivered to the appropriate cell type, thereby increasing the concentration siRNA within the cell type.

In other embodiments of the present invention, an appropriate cell or tissue is provided with expression construct which comprises a nucleic acid that encodes one or both strands of an siRNA that is specific to the gene and/or other nucleic acid which encodes the NF-HEV polypeptide or a biologically active fragment thereof. In these embodiments, the nucleic acid that encodes one or both strands of the siRNA can be placed under the control of either a constitutive or a regulatable promoter. In some embodiments, the nucleic acid encodes an siRNA that forms a hairpin structure.

Inhibition of gene expression refers to the absence or reduction (observable decrease) in the level of protein and/or mRNA product from the gene and/or other nucleic acid which encodes the NF-HEV polypeptide or a biologically active fragment thereof. The consequences of inhibition can be confirmed by examination of the outward properties of the cell or organism, such as reduction in inflammation, by biochemical techniques, such as the quantitation of pro-inflammatory chemokines or by directly measuring levels of the transcript of the gene and/or other nucleic acid which encodes the NF-HEV polypeptide or a biologically active fragment thereof. For a cell line or whole organism, in some embodiments, gene expression is assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin.

Depending on the assay, quantitation of the amount of gene expression allows one to determine a degree of inhibition which is greater than 10%, 33%, 50%, 90%, 95% or 99% as compared to an untreated cell. Lower doses of injected material and longer times after administration of the antisense nucleic acid or siRNA may result in decreased inhibition or partial inhibition of the expression of the gene and/or other nucleic acid which encodes the NF-HEV polypeptide or a biologically active fragment thereof.

Antisense nucleic acids and siRNAs comprising a nucleotide sequences identical to a portion of a gene and/or other nucleic acid which encodes the NF-HEV polypeptide or a biologically active fragment thereof are contemplated in some embodiments of the present invention. However, nucleic acid sequences with insertions, deletions, and single point mutations relative to the target sequence are also effective for inhibition of gene expression. Thus, sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the siRNA and the portion of the target gene is preferred. Alternatively, the duplex region of the RNA may be defined functionally as a nucleotide sequence that is capable of hybridizing with a portion of the target gene transcript. Exemplary hybridization conditions are 400 mM NaCl, 40 mM PIPES pH 6.4, 1 mM EDTA, 50° C. or 70° C. hybridization for 12-16 hours; followed by washing.

Modulation of Chemokine Level and Activity by NF-HEV

Some embodiments of the present invention relate to the modulation of chemokine level or activity by modulating the level or activity of the NF-HEV polypeptide or a biologically active fragment thereof. In some embodiments, increasing the level or activity of the NF-HEV polypeptide or a biologically active fragment thereof causes an increase or a decrease in the level or activity of chemokines. In other embodiments, decreasing the level or activity of the NF-HEV polypeptide or a biologically active fragment thereof causes a decrease or an increase in the level of chemokines. Chemokines can include, but are not limited to, XCL1, XCL2, CCL1, CCL2, CCL3, CCL3L1, SCYA3L2, CCL4, CCL4L, CCL5, CCL6, CCL7, CCL8, SCYA9, SCYA10, CCL11, SCYA12, CCL13, CCL14, CCL15, CCL16, CCL17, CCL18, CCL19, CCL20, CCL21, CCL22, CCL23, CCL24, CCL25, CCL26, CCL27, CCL28, clone 391, CARP CC-1, CCL1, CK-1, regakine-1, K203, CXCL1, CXCL1P, CXCL2, CXCL3, PF4, PF4V1, CXCL5, CXCL6, PPBP, SPBPBP, IL8, CXCL9, CXCL10, CXCL11, CXCL12, CXCL14, CXCL15, CXCL16, NAP-4, LFCA-1, Scyba, JSC, VHSV-induced protein, CX3CL1 and fCL1. In some embodiments, modulating the level or activity of the NF-HEV polypeptide or a biologically active fragment thereof modulates the physiological effect of a chemokine. Such physiological effect can result from, for example, modulating of the transcription of a nucleic acid encoding a chemokine or from modulating the interaction of the chemokine with its receptor or with another molecule such as a transcription factor. Chemokine receptors can include, but are not limited to, CCR1, CCR2, CCR3, CCR4, CCR5, CCR6, CCR7, CCR8, CCR9, CCR10, CCR11, CXCR1, CXCR2, CXCR3, CXCR4 and CXCR5.

In certain embodiments, the level or activity of one or more pro-inflammatory chemokines is modulated by modulating the level or activity of the NF-HEV polypeptide or a biologically active fragment thereof. In some embodiments, an increased level or activity of the NF-HEV polypeptide or a biologically active fragment thereof causes an increase in the level and/or activity of pro-inflammatory chemolines. In other embodiments, decreased level or activity of the NF-HEV polypeptide or a biologically active fragment thereof causes a decrease in the expression and/or activity of pro-inflammatory chemolines. Such pro-inflammatory chemokines include, but are not limited to, CXCL1/GROα, CXCL2/GROβ, CXCL6, CXCL8/IL8 and CCL2/MCP1.

Some embodiments of the invention relate to methods of modulating the level or activity of pro-inflammatory chemokines by using molecules that do not modulate the level or activity of pro-inflammatory cytokines. In some embodiments, such molecules decrease the level or activity of the NF-HEV polypeptide or a biologically active fragment thereof. Examples of molecules having the ability to inhibit the production of NF-HEV polypeptide include, but are not limited to, antisense nucleic acids and siRNAs.

In other embodiments, the level or activity of cellular adhesion molecules are modulated by modulating the level or activity of the NF-HEV polypeptide or a biologically active fragment thereof. For example, increasing the level or activity of the NF-HEV polypeptide or a biologically active fragment thereof increases the level or activity of ICAM1.

It will be appreciated by one skilled in the art that the above-described methods of modulating level or activity of the NF-HEV polypeptide or a biologically active fragment thereof can occur both in vivo and in vitro. In some embodiments, the above-described methods occur in mammalian HEVEC cells. In certain embodiments the HEVEC cells are human cells.

Modulation of Inflammation by Modulating the Activity of NF-HEV

Some embodiments of the present invention relate to ameliorating the symptoms associated with an inflammatory condition by modulating the level or activity of the NF-HEV polypeptide or a biologically active fragment thereof in cells. In some embodiments, the cells are HEVECs. In some embodiments, the cells are from a mammal. In other embodiments the cells are human cells. The inflammatory conditions that are modulated can include, but are not limited to, rheumatoid arthritis and inflammatory bowel disease (ulcerative colitis and/or Crohn's disease).

In some embodiments, the level or activity of the NF-HEV polypeptide or a biologically active fragment thereof can be modulated by supplying a compound which modulates the level or activity of the NF-HEV polypeptide or a biologically active fragment thereof either directly or indirectly. For example, in some embodiments, supplying a molecule such as an antisense nucleic acid or an siRNA effectively modulates the level or activity of the NF-HEV polypeptide or a biologically active fragment thereof.

In other embodiments of the present invention, the symptoms of a condition associated with inflammation are ameliorated by identifying a subject suffering from an inflammatory condition then modulating the level or activity of the NF-HEV polypeptide or a biologically active fragment thereof in the subject. In some embodiments, the subject is a human.

Some embodiments of the present invention relate to methods of ameliorating the symptoms associated with an inflammatory condition by using molecules that do not modulate the level or activity of pro-inflammatory cytokines. In some embodiments, such molecules decrease the level or activity of the NF-HEV polypeptide or a biologically active fragment thereof. Examples of molecules having the ability to inhibit the production of NF-HEV polypeptide include, but are not limited to, antisense nucleic acids and siRNAs.

In some embodiments of the present invention, the symptoms of the inflammatory disease are reduced by modulating the level of transcription of at least one promoter that is responsive to an NF-HEV polypeptide or a biologically active fragment thereof. In some embodiments, the promoter controls the expression of a pro-inflammatory chemokine or another pro-inflammatory molecule.

Having generally described this invention, a further understanding can be obtained by reference to certain specific examples which are provided herein for purposes of illustration only, and are not intended to be limiting unless otherwise specified.

### EXAMPLES

An important element in the cloning of the NF-HEV cDNA from HEVECs was the development of protocols for obtaining HEVECs RNA, since HEVECs are not capable of maintaining their phenotype outside of their native environment for more than a few days. Total RNA was obtained from HEVECs freshly purified from human tonsils. Highly purified HEVECs were obtained by a combination of mechanical and enzymatic procedures, immunomagnetic depletion and positive selection (Girard and Springer (1995) Immunity 2:113-123) (Baekkevold et al. (1999) Lab Invest 79:327-36). Tonsils were minced finely with scissors on a steel screen, digested with collagenase/dispase enzyme mix and unwanted contaminating cells were then depleted using immunomagnetic depletion. HEVECs were then selected by immunomagnetic positive selection with magnetic beads conjugated to the HEV-specific antibody MECA-79 (Girard and Springer (1995) Immunity 2:113-123) (Baekkevold et al. (1999) Lab Invest 79:327-36).

Despite these methods, extensive molecular characterization of the HEVEC phenotype is still hampered by the low number of cells available after purification, thereby ruling out traditional subtraction cloning techniques, which typically require several micrograms of mRNA (Byers et al. (2000) Int J Exp Pathol 81:391-404). To overcome this problem, we previously adapted the PCR-based method of suppression subtractive hybridization (SSH) (Diatchenko et al. (1996) Proc Natl Acad Sci USA 93:6025-30) to identify genes preferentially expressed in human tonsillar HEVECs compared with human umbilical vein endothelial cells (HUVCs) (Girard et al. (1999) Am J Pathol 155:2043-55). With this method we generated a subtracted HEVEC cDNA library from 1 μg of total RNA, and were able to clone several HEV-expressed cDNAs, including the promiscuous chemokine receptor DARC, mitochondrial genes, and secreted extracellular matrix (ECM) proteins, such as mac25/IGFBP7/angiomodulin (Girard et al. (1999) Am J Pathol 155:2043-55). Thus, we showed that SSH could be applied for cloning of differentiation-specific genes from a very limited starting material. This strategy has since been applied for characterization of ECs from several other vascular beds Kirsch et al. (2001) Brain Res 910:106-15; Stier et al. (2000) FEBS Lett 467:299-304; Wang et al. (2001) Stroke 32:1020-7). SSH was also recently utilized to clone the novel vascular endothelial junction-associated molecule (VE-JAM) from an HEVEC cDNA library (Palmeri et al. (2000) J Biol Chem 275:19139-45).

To be reliable, SSH requires a low but significant enrichment of genes in the cells of interest compared with those used for subtraction. Therefore, to identify differentiation-specific genes from HEVECs, subtraction was not performed with HUVECs but with the more closely related and truly microvascular nasal polyp-derived microvascular endothelial cells (PMECs) (Jahnsen et al. (1997) Am J Pathol 150:2113-23). This strategy allowed us to identify, in addition to the matricellular protein hevin (which validated our approach), a nuclear factor preferentially expressed in HEVECs, designated nuclear factor from HEV (NF-HEV). NF-HEV mRNA was detected by in situ hybridization in HEVs from several human lymphoid tissues, including tonsils, Peyer's patches and mesenteric lymph nodes. Virtual northern and western blot analysis revealed preferential expression of NF-HEV in HEVECs, compared to two other types of ECs, namely microvascular PMECs and macrovascular HUVECs. NF-HEV exhibits a consensus bipartite nuclear localization sequence and localized to the nucleus when ectopically expressed in HUVECs. Immunohistochemistry performed on human tonsil sections showed a similar in situ nuclear localization of NF-HEV in HEVECs. Additionally, threading and molecular modeling analyses suggested that NF-HEV contains an homeodomain-like HTH DNA-binding domain (DBD) in its amino-terminal part. Together, our results characterized the first nuclear factor preferentially expressed in HEVECs that is likely to play a key role in the control of the specialized HEV phenotype.

Further studies characterized the role of NF-HEV in inflammation. For example, in situ hybridization studies revealed that NF-HEV is expressed in endothelial cells from small blood vessels in Crohn's disease and rheumatoid arthritis. Real time PCR experiments showed that NF-HEV mRNA is induced by pro-inflammatory cytokines. Microarray and real time PCR analyses revealed NF-HEV supplied to HUEC cells on a retroviral vector induced the expression of pro-inflammatory chemokines. The results of the microarray analysis was confirmed using a variety of supplemental techniques such as ELISA and immunofluorescence staining. These results demonstrate that NF-HEV is a modulator of inflammation.

### Example 1

### Suppression Subtractive Hybridization (SSH)

To identify cDNAs preferentially expressed in HEVEC, a PCR Select library was generated from HEVEC cDNA subtracted against PMEC cDNA (HEVEC_{-PMEC}). MECA-79-positive HEVECs were purified from human tonsils and PMECs were isolated from nasal polyps. SSH was performed as described (Girard et al. (1999) Am J Pathol 155:2043-55) with some modifications. Total RNA was isolated from highly purified HEVECs (Baekkevold et al. (1999) Lab Invest 79:327-36) cultured for 2 days with an RNeasy kit (Qiagen). PMECs were prepared from nasal polyps as described (Jahnsen et al. (1997) Am J Pathol 150:2113-23), stained with anti-CD34-FITC (Diatec), and purified by cell sorting (FACSVantage, Becton Dickinson). PMEC mRNA was isolated by μMACS mRNA isolation kit (Miltenyi Biotech). To obtain sufficient amounts of double-stranded (ds) cDNA for subtraction, both PMEC and HEVEC cDNAs were preamplified with the SMART PCR cDNA synthesis kit (Clontech). cDNAs synthesized from 1 μg of total RNA (HEVECs) or 0.15 μg mRNA (PMECs) with Advantage KlenTaq polymerase (22 cycles, Clontech) were used with the PCR Select cDNA subtraction kit (Clontech). Briefly, PCR-generated HEVEC and PMEC cDNAs were digested with RsaI (New England Biolabs) and ligated to ds cDNA adaptors. For the first hybridization, the mixtures of HEVEC and PMEC cDNAs were incubated for 8 hours at 68° C. For the second hybridization, excess PMEC cDNA was added and incubated for 22 hours at 68° C. Differentially expressed cDNAs were then selectively amplified by two successive PCR (27 cycles) and nested PCR (10 cycles) reactions.

T/A cloning libraries of the subtracted cDNAs were prepared as described (von Stein et al. (1997) Nucleic Acids Res 25:2598-602). Briefly, the HEVEC_{-PMEC} and PMC_{-HEVEC} subtracted mixtures (200 ng) were cloned directly into pCR2.1-TOPO (TA Cloning kit, Invitrogen) and introduced into One Shot Competent TOP10 cells (Invitrogen) according to the manufacturer's instructions. The bacteria were plated on agar plates containing 100 μg/ml ampicillin, 100 μM isopropyl-β-D-thiogalactoside (IPTG) and 50 μg/ml X-Gal, and then grown until blue/white colonies appeared.

A total of 960 clones were obtained in the PCR-select HEVEC_{-PMEC} cDNA library.

### Example 2

### Differential Hybridization Screening with Subtracted Probes

A total of 960 individual recombinant (white) colonies were picked and used to inoculate ten 96-well microtitre plates with LB medium and 100 μg/ml ampicillin, which was incubated overnight and diluted 1:4 with H₂O. This diluted bacterial culture (1 μl) was used to PCR amplify cloned inserts in 25 μl reactions with M13rev and M13for (−20) primers flanking the vector cloning site under the following conditions: 95° C. for 5 min and then 30 cycles of the following temperature/time sequence: 94° C. for 30 sec, 55° C. for 30 sec, and 72° C. for 1 min. The PCR reaction products (12 μl) were then loaded onto duplicate agarose gels (1.6% w/v), denatured, and blotted onto nylon membranes. The filters were hybridized with equivalent amounts of ³²P-labeled cDNA of similar specific activity derived from HEVEC and PMEC total RNA as described in Example 1.

Differential screening of these 960 clones with radioactive probes generated from HEVEC or PMEC cDNAs, revealed 49 cDNAs preferentially expressed in HEVECs.

### Example 3

### Sequence Analysis of Differentially Expressed Genes

Miniprep DNA of the differentially hybridizing clones form Example 2 was prepared and sequenced at Medigenomix (Marti ed, Germany) with the plasmid-specific TOPO1 and TOPO2 oligonucleotides.

Sequencing of these cDNAs showed that the most abundant family of genes was mitochondrial enzymes (12 clones), particularly transcripts for cytochrome c oxidase 1. This was in line with our previous report (Girard et al. (1999) Am J Pathol 155:2043-55) that HEVECs express higher levels of these enzymes than other ECs. Our screen also identified three independent clones corresponding to the secreted matricellular protein hevin, one of the known markers of tonsillar HEVECs (Girard et al. (1999) Am J Pathol 155:2043-55; Girard and Springer (1995) Immunity 2:113-123). Using two distinct polyclonal antisera, we confirmed preferential expression of hevin in MECA-79-positive-HEVECs from human tonsils, as well as MadCAM-1-positive-HEVECs from human Peyer's patches. In addition to the hevin clones, which validated our HEVEC_{-PMEC} SSH approach, we identified several other cDNAs corresponding to previously characterized genes, including endothelial multimerin (4 clones), which is a secreted homomultimeric factor V-binding protein (Hayward et al. (1998) Blood 91:1304-17), the complement inhibitor CD59 (2 clones), and the Nck adaptor protein NCK1 (2 clones). Abundant expression of multimerin and CD59 in HEVECs was confirmed by immunohistochemistry on human tonsil sections.

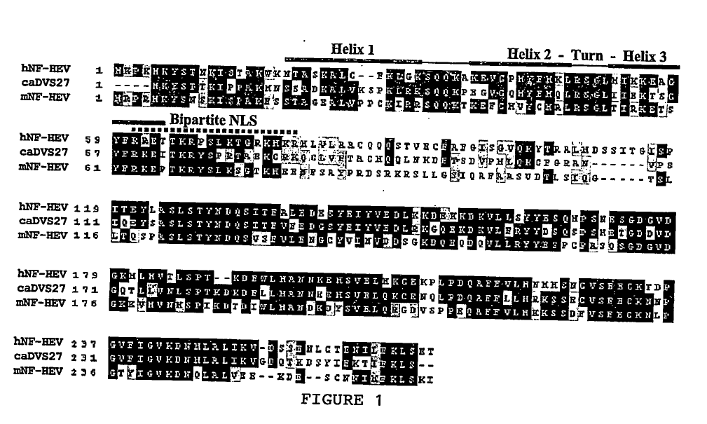

Among the sequences corresponding to human genes not yet characterized, we focused on one that was represented by four distinct cDNA clones within the HEVEC_{-PMEC} library. Sequencing of these four cDNA clones revealed a sequence identical to that of a human cDNA deposited in Genbank with the annotation “Homo sapiens mRNA for DVS27-related protein” (GenBank Acc. AB024518). We termed the protein encoded by this cFNA NF-HEV. The NF-HEV cDNA appears to encode a putative human ortholog of the canine DVS27 protein, previously identified in a screen for genes differentially expressed in canine vasospastic cerebral arteries after subarachnoid hemorrhage (Onda et al. (1999) J Cereb Blood Flow Metab 19:1279-88). Databases searches with both the nucleotide and amino acid sequences of canine DVS27 (Genbank Acc. AB024517), using the programs BLASTN, TBLASTN and BLASTP (GenBank non-redundant, human htgs and human EST databases at National Center for Biotechnology Information, which can be accessed by typing the following, “http://www.ncbi.nlm” into the address bar of a web browser followed immediately by “.nih.gov”), failed to reveal any other human cDNA or protein more closely related to DVS27 than NF-HEV. This further suggested that human NF-HEV is an ortholog of canine DVS27. Two murine cDNAs encoding a putative mouse ortholog of human NF-HEV (GenBank Acc. XM_{—}123362 and NM_{—}133775) were also identified by searching GenBank databases with the human NF-HEV sequence. Alignment of the human and mouse NF-HEV proteins (48% identity over 270 residues) with the canine DVS27 sequence (56% identity between HNF-HEV and canine DVS27) revealed that the NF-HEV/DVS27 protein is composed of two evolutionary conserved regions separated by a highly divergent linker region in the central part (FIG. 1).

### Example 4

### Genomic Structure of NF-HEV

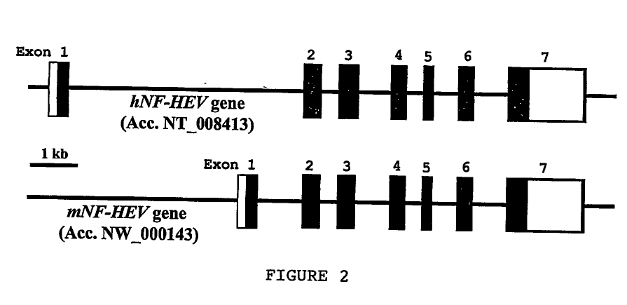

The structure of the human NF-HEV gene was determined by sequence analysis using BLAST to search the nonredundant (NR) sequence database at NCBI. Human NF-HEV cDNA or protein sequences as query sequences, revealed a genomic hit from the Homo Sapiens chromosome 9 sequence (GenBank Acc. NT-008413) that covered the whole NF-HEV cDNA. This genomic contig contains three independent UniSTS (UniSTS entries: SHGC-15129, stSG27179, RH101248) that have been previously mapped at 9p24.1, between microsatellite markers D9S 178 and D9S 168. This suggested that the human NF-HEV gene is located on the short arm of chromosome 9 at 9p24.1. Alignment between the NF-HEV cDNA and genomic sequences revealed that there are 7 exons that span more than 16 kb of genomic DNA (FIG. 2). All the exon-intron boundaries followed the GT-AG rule. The human NF-HEV gene shared a similar organization with its mouse ortholog (FIG. 2), that we identified in a Mus musculus genomic contig (GenBank Acc. NW_{—}000143). The size of exons were found to be strictly conserved between the two species, with the exception of exon 3 that contains 15 additional nucleotides in the human sequence, corresponding to an insertion of 5 residues in the middle of the human NF-HEV protein (FIG. 1).

### Example 5

### In Situ Hybridization

To assess the tissue distribution of this gene, we performed in situ hybridization using riboprobes corresponding to the open reading frame of NF-HEV.

In situ mRNA hybridization was performed as described (Hashimoto et al. (2000) Blood 96:2206-14). Briefly, digoxigenin-labeled riboprobes were generated from the NF-HEV cDNA with the DIG RNA labeling kit according to the manufacturer's directions (Boehringer Mannheim, Mannheim, Germany). Frozen sections (8 μm) from human palatine tonsils, Peyer's patches and mesenteric lymph nodes were fixed in 4% paraformaldehyde (PFA)/DEPC-treated PBS and subsequently washed in PBS containing 0.1% active DEPC (Sigma). After equilibration in 5×SSC, sections were prehybridized (2 h, 59° C.) in hybridization solution (50% formamide, 5×SSC, 50 μg/ml yeast tRNA, 100 μg/ml heparin, 1× Denhardt solution, 0.1% Tween 20, 0.1% CHAPS, and 5 mM EDTA). Sections were subsequently hybridized overnight at 59° C. with 250 ng/ml of riboprobe in hybridization solution. High stringency wash was performed, and the sections were next incubated (45 min) with horseradish peroxidase (HRP)-conjugated rabbit anti-DIG (1:50; DAKO, Glostrup, Denmark) in blocking buffer (0.1% Boehringer Blocking Agent dissolved in Tris-HCl 100 mM, NaCl 150 mM, pH 7.5), followed by signal amplification with biotin-tyramide deposition (GenPoint kit; DAKO). Subsequently, sections were incubated (20 min) with HRP-conjugated rabbit anti-biotin (1:50 in blocking buffer; DAKO), followed by an additional cycle of biotin-tyramide deposition. Signal was detected by incubation (20 min) with alkaline phosphatase (AP)-conjugated rabbit anti-biotin (1:50 in blocking buffer; DAKO), followed by the AP substrate Fast Red (Ventana Medical Systems, Tucson, Ariz.). Finally, the sections were counterstained with hematoxylin.

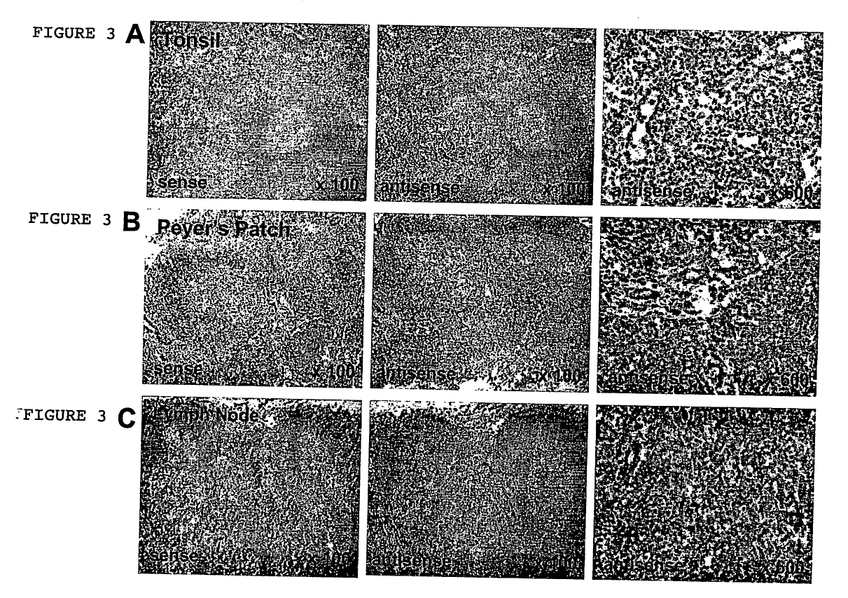

Strikingly, the antisense riboprobe hybridized strongly to HEVs in the T-cell zones of human tonsil (FIG. 3A), Peyer's patch (FIG. 3B), and mesenteric lymph node (FIG. 3C). Indeed, higher magnification clearly revealed hybridization signals within HEVECs as well as in scattered cells in the T-cell zone (arrow heads). Hybridization with a sense probe produced no signal. These confirmed preferential expression of this gene in human HEVs in vivo. Experiments described below confirm the localization of the corresponding protein in the cell nucleus.

### Example 6

### Virtual Northern Blot and Western Blot Analysis

Preferential expression of NF-HEV in human HEVECs was confirmed by virtual northern and western blot analyses. cDNAs from HEVECs, PMECs, first passage HUVECs and placenta were generated from total RNA using SMART PCR. The PCR products were electrophoresed on 1.6% agarose gels (0.5 μg per lane), transferred onto nylon filters and hybridized as described (Girard et al. (1999) Am J Pathol 155:2043-55) with a ³²P-labeled cDNA probe corresponding to the coding region of NF-HEV.

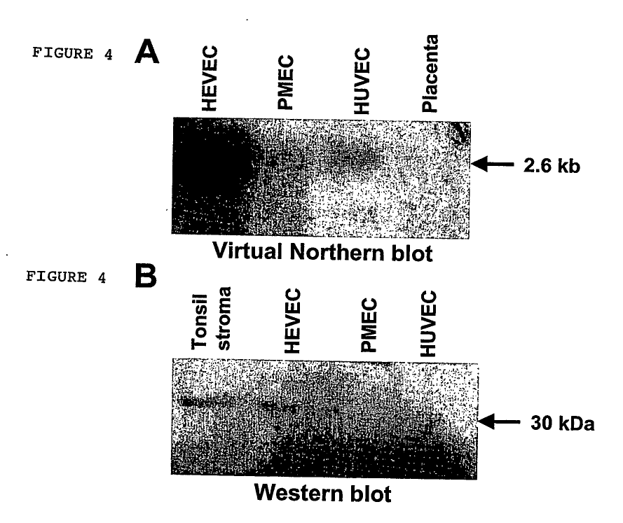

Analysis of the radiolabeling revealed a prominent band of ˜2.6 kb in HEVECs (FIG. 4A, lane 1) which agreed well with the size of NF-HEV mRNA. Conversely, this 2.6-kb signal was detected at only very low levels in PMECs and HUVECs and was almost undetectable in placenta (FIG. 4A, lane 4).

Western blot analysis was used to confirm the preferential expression of NF-HEV in HEVECs at the protein level. Lysates from purified HEVECs and primary cultures of PMECs and HUVECs (each corresponding to ˜10⁵ cells) were fractionated by sodium dodecyl sulfate-polyacrylamide gel electrophoresis (SDS-PAGE) (10%). Detection was performed with rabbit antiserum to NF-HEV (1:500), followed by HRP-conjugated donkey anti-rabbit Ig (1:1000; Amersham), and finally an enhanced chemiluminescence kit (Pierce).

The antibodies recognized a ˜30 kD protein in lysates from tonsil stroma and purified HEVECs, but not in PMECs or HUVECs (FIG. 4B). The apparent molecular weight of ˜30 kD for endogenous NF-HEV was in agreement with the predicted M_{w} of 31 kD and the size of a recombinant NF-HEV protein produced in Escherichia coli.

### Example 7

### Epitope Tagging and Immunofluorescence

Because the predicted NF-HEV amino acid sequence contains a consensus bipartite nuclear localization sequence (NLS, Prosite PS00015), near the linker region (FIG. 1), we decided to investigate whether NF-HEV could localize to the nucleus of ECs. For that purpose, we designed an expression construct with NF-HEV fused to the c-myc epitope tag, which was transfected into primary HUVECs and detected by indirect immunofluorescence staining with antibodies to myc.

An epitope tagged vector comprising NF-HEV was constructed by cloning the coding region of NF-HEV into the vector PcDNA3.1A/myc-his (Invitrogen). First, the open reading frame of NF-HEV was amplified by PCR using primers 5′-GAATTCTGAAAAATGAAGCCTAAAATGAAGTATTCAAC-3′ (SEQ ID NO: 9) and 5′-GGGCCCAGTTTCAGAGAGCTTAAACAAGATATTTCAG-3′ (SEQ ID NO: 10). The product was digested with EcoRI and ApaI and then cloned in frame with the myc tag of the PcDNA3.1A.

HUVECs were grown in ECGM medium (Promocell) and HeLa cells were grown in Dulbecco's Modified Eagle's Medium supplemented with 10% fetal calf serum and 1% penicillin-streptomycin (all from Gibco-BRL). HUVECs were plated on coverslips in RPMI medium and transiently transfected with 0.7 μg PcDNA3.1A-NF-HEV-myc-his expression vector and Genejammer transfection reagent according to the manufacturer's instructions (Stratagene). HeLa cells were plated on coverslips and transiently transfected with 2 μg PcDNA3.1A-NF-HEV-myc-his expression vector, with the calcium phosphate method. After medium change, transfected cells were incubated for 48 h to allow gene expression and then washed twice with PBS, fixed for 15 min at room temperature in PBS containing 3.7% PFA, and washed again with PBS prior to neutralization with 50 mM NH₄Cl in PBS for 5 min at room temperature. Cells were permeabilized for 5 min at room temperature in PBS containing 0.1% Triton-X100, and washed twice with PBS. Permeabilized cells were then incubated for 2 hr at room temperature with an anti-myc monoclonal antibody (IgG, 7 μg/ml, Clontech) in PBS with 1% (w/v) bovine serum albumin (BSA). Cells were then washed three times 5 min at room temperature in PBS-BSA, and incubated for 1 hr with FITC-labeled rabbit anti-mouse IgG (1:40, Amersham Pharmacia Biotech). After extensive washing in PBS, samples were air dried and mounted in Mowiol (Hoechst Pharmaceuticals). Fluorescence of fixed immunostained cells was viewed with a Leica confocal laser-scanning microscope.

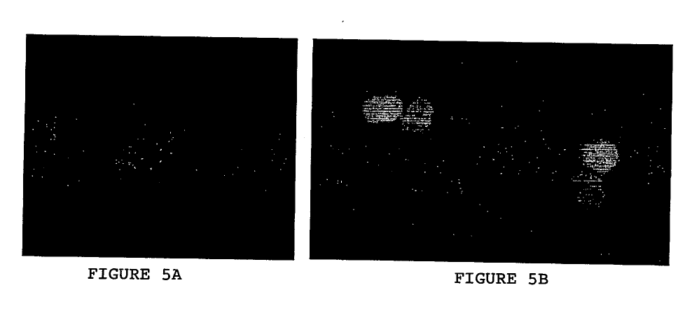

Confocal immunofluorescence microscopy revealed a strict intranuclear localization of the epitope-tagged NF-HEV (FIG. 5A). The myc-tagged-NF-HEV protein also localized to the nucleus when ectopically expressed in HeLa cells (FIG. 5B), suggesting that the nuclear localization of NF-HEV is not a specific property of ECs.

### Example 8

### Antibody Production, Immunohistochemistry, and Western Blotting

To determine the subcellular localization of NF-HEV in HEVECs in situ, we performed immunohistochemistry with the rabbit antibodies raised against NF-HEV peptides.

Rabbit polyclonal antibodies were raised against the peptides MKPKMKYSTNKISTAC (SEQ ID NO: 11) and CYFRRETTKRPSLKTG (SEQ ID NO: 12), corresponding to amino acids 1-15 and 58-73 of the human NF-HEV sequence, respectively, using multiple antigen peptides technology (Eurogentec). The antisera were applied in immunohistochemistry as previously described (Girard et al. (1999) Am J Pathol 155:2043-55). In brief, acetone-fixed sections (4 μm) of human palatine tonsils were first incubated with a mixture of mAb MECA-79 (rat IgM, 1:30; courtesy of E. C. Butcher, Stanford, Calif.) and anti-NF-HEV rabbit antiserum (1:1000), followed by a mixture of Cy3-conjugated goat anti-rat IgM (1:200; Jackson ImmunoResearch) and Alexa Fluor 488-conjugated goat anti-rabbit IgG (Molecular Probes). The sections were mounted with 4,6-diamidino-2-phenylindole (DAPI)-containing Vectashield (Vector). Negative controls were tissue sections incubated with concentration-matched irrelevant rat IgM and preimmune rabbit serum.

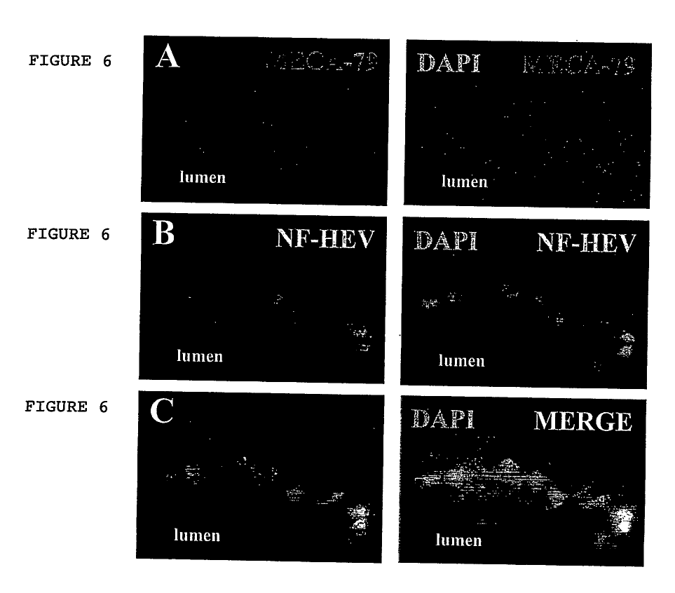

Immunostaining of human tonsil sections with the above-described reagents demonstrated strong NF-HEV expression in MECA-79-positive HEVs (FIG. 6A-C, left panels). Costaining of nuclear DNA with DAPI further showed that NF-HEV was concentrated in the nucleus of HEVECs (FIG. 6A-C, right panels). Lower magnification also revealed that the antibodies to NF-HEV decorated, scattered single cells (the identity of which remains unknown) in the T cell zone (data not shown), in addition to the MECA-79-positive-HEVs. This result was consistent with the in situ hybridization results described above (FIG. 3A-C). In conclusion, although NF-HEV expression did not appear to be strictly HEV-specific, our immunohistochemistry data clearly revealed abundant in vivo expression of NF-HEV in the nucleus of HEVECs.

### Example 9

### Threading and Molecular Modelling Analyses

Searches in Prosite and Pfam databases with the NF-HEV sequence failed to reveal significant similarities to previously characterized protein sequence motifs, excepted for a low level of homology of the NF-HEV amino-acids 28 to 68 with prokaryotic Helix-Turn-Helix (HTH) DBDs (HTH_ARAC, Prosite PS00041). Because detection of sequence homology is more sensitive and selective when aided by secondary structure information, structural homologs of human NF-HEV in the PDB crystallographic database, were searched with the SeqFold threading program (Olszewslci (1999) Theor. Chem. Acc. 101:57-61) which combines sequence and secondary structure alignment.

In particular, for this modelling analysis, we used the InsightII, SeqFold, Homology and Discover modules from the Accelrys (San Diego, Calif.) molecular modelling software (version 98), run on a Silicon Graphics O2 workstation. Structural homologs of human NF-HEV were searched with the SeqFold threading program (Olszewski (1999) Theor. Chem. Acc. 101:57-61), which combines sequence and secondary structure alignment. Optimal secondary structure prediction of the query protein domains was ensured by the DSC method (King and Sternberg (1996) Protein Sci 5:2298-310) within SeqFold. The engrailed homeodomain (PDB code: 1DU0) was identified as the best structural template of the NF-HEV amino-terminal domain (NF-HEV aa 1-65). We used the threading-derived secondary structure alignments as input for homology-modelling, which was performed according to a previously described protocol. (Manival et al. (2001) Nucleic Acids Res 29:2223-33) The validity of the models was checked both by Ramachandran analysis and folding consistency verification as previously reported. (Manival et al. (2001) Nucleic Acids Res 29:2223-33)

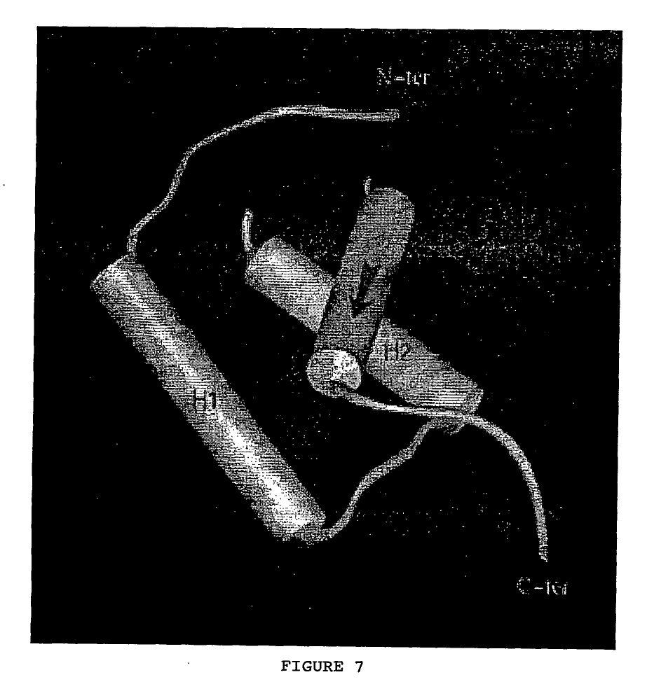

This search revealed significant structural homologies between the first 65 amino-terminal residues of NF-HEV and the DNA-binding homeodomains of several drosophila (engrailed, fushi-tarazu, . . . ) and vertebrate (POU, . . . ) transcription factors. The crystallographic structure (DB # 1DU0) of the drosophila transcription factor engrailed homeodomain (Grant et al. (2000) Biochemistry 39:8187-8192) provided the best score of the search. The resulting threading-derived secondary structure alignment, was used to generate a homology-based model for the amino-terminal domain of human NF-HEV (FIG. 7). Similarly to the homeodomain Kissinger et al. (1990) Cell 63:579-90) and various other eukaryotic HTH DBDs (human centromere protein CENP-B, human Myb transcription factor, yeast telomere binding protein RAP1), (Iwahara et al. (1998) EMBO J. 17:827-37) NF-HEV was predicted to contain a homeodomain-like HHTH motif that could be described as a right-handed three-helical bundle, (Grant et al. (2000) Biochemistry 39:8187-8192; Kissinger et al. (1990) Cell 63:579-90) composed of an hydrophobic core of two α-helices (helices 2 and 3 corresponding to the HTH motif) completed by another N-terminal α-helix (helix 1). A conserved characteristic of this HHTH motif is the packing of α-helices 2 and 3 at nearly a right angle to each other (FIG. 7); the turn between α-helices 2 and 3 offsets α-helix 3 so that the N-terminal part of al-helix 3, which is predicted to bind to the target DNA major groove, is packed against the middle of α-helix 2. Together, our threading and modeling results suggested that the amino-terminal part of NF-HEV (aa 1-65) corresponds to a novel homeodomain-like HTH DBD.

### Example 10

### RT-PCR Analysis

For RT-PCR analysis, total RNA was purified from human tonsil HEVEC, rheumatoid arthritis ECs, Crohn's disease ECs, Colon tumor ECs or HeLa cells. Human tonsil HEVEC, rheumatoid arthritis ECs, Crohn's disease ECs, Colon tumor ECs were purified from human tissues using a combination of mechanical and enzymatic procedures, immunomagnetic depletion and positive selection (Girard and Springer (1995) Immunity 2:113-123) (Baekkevold et al. (1999) Lab Invest 79:327-36). Fresh human tissues were minced finely with scissors on a steel screen, digested with collagenase/dispase enzyme mix and unwanted contaminating cells were then depleted using immunomagnetic depletion. ECs were then selected by immunomagnetic positive selection with magnetic beads conjugated to EC-specific antibody (Girard and Springer (1995) Immunity 2:113-123) (Baekkevold et al. (1999) Lab Invest 79:327-36). Two micrograms of human tonsil HEVEC, rheumatoid arthritis ECs, Crohn's disease ECs, Colon tumor ECs or Hela cells total RNA was reverse transcribed using Superscript™ first strand synthesis system from Invitrogen. One twentieth of the reaction products were subjected to PCR amplification with 10 pmol of primers using Advantage2 polymerase mix according to the supplier's instructions in a 25 μl reaction volume with 30 cycles (94° C. for 15 sec, 60° C. for 20 sec, and 72° C. for 1 min). The following NF-HEV-gene specific primers were used: NF-HEV-1: 5′-CACCCCTCAAATGAATCAGG 3′ (SEQ ID NO: 13) and NF-HEV-2: 5′-GGAGCTCCACAGAGTGTTCC 3′ (SEQ ID NO: 14). The following G3PDH-gene specific primers were used: G3PDH-1: 5′-ACCACAGTCCATGCCATCAC 3′ (SEQ ID NO: 15) and G3PDH-2: 5′-TCCACCACCCTGTTGCTGTA 3′ (SEQ ID NO: 16).

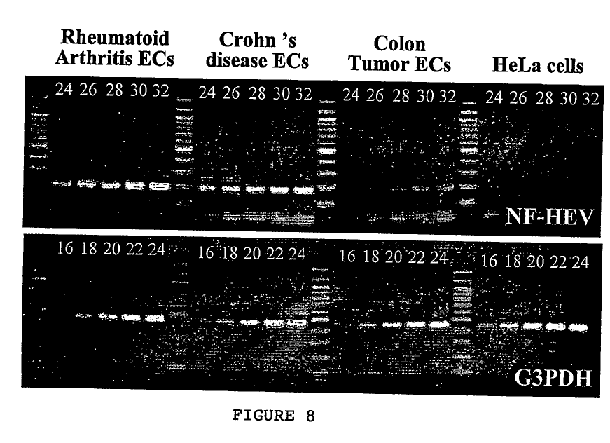

All PCR reactions were done at the same time and the identity of the PCR products was confirmed by restriction mapping or sequencing. Whereas the transcript for the metabolic enzyme G3PDH was present in every cell type (as expected), the transcript for NF-HEV was only present in HEVECs associated with rheumatoid arthritis and those associated with Crohn's disease (FIG. 8).

### Example 11

### In Vitro Assay to Identify NF-HEV Polypeptide DNA Targets

DNA binding specificity of NF-HEV is determined using a random oligonucleotide selection method allowing unbiased analysis of binding sites selected by the NF-HEV protein from a random pool of possible sites. The method is carried out essentially as described in Pollack and Treisman (1990), A sensitive method for the determination of protein-DNA binding specificities. Nuc. Acid Res. 18:6197-6204. Also, see (Blackwell and Weintraub, (1990) Science 250: 1104-1110; Ko and Engel, (1993) Mol. Cell. Biol. 13:4011-4022; Merika and Orkin, (1993) Mol. Cell. Biol. 13: 3999-4010; and Krueger and Morimoto, (1994) Mol. Cell. Biol. 14:7592-7603).

Random Oligonucleotide Selection

According to the protocol of Pollack and Treisman (1990), supra, a 77 bp oligonucleotide having sequences as follows is synthesized: 5′-CAGGTCAGTTCAGCGGATCCTGTCG-(N)₂₇-GAGGCGAATTCAGTGCAACTGCAGC-3′ (SEQ ID NO: 17), where N is any nucleotide, and primers complementary to each end. Primer P is: 5′-GCTGCAGTTGCACTGAATTCGCCTG-3′ (SEQ ID NO: 18), and primer R is 5′-CAGGTCAGTTCAGCGGATCCTGTCG-3′ (SEQ ID NO: 19).

The 77-mer is purified on an 8% denaturing acrylamide gel and used to prepare a probe for gel shift analysis. The 77-mer oligonucleotide is labeled and made double stranded with Klenow fragment in the presence of [α-12P]dCTP. Approximately 5 ng of labeled probe and 1 μg of poly(dI-dC) is mixed with 10 nM NF-HEV protein or a portion thereof and incubated at 25° C. for 30 min. The extended binding reaction permits proteins to cycle through several association and dissociation events, leading to the isolation of higher-affinity selected sequences. The binding reaction mixture is then subjected to electrophoresis on a 4% (40:1) acrylamide gel in 0.25×Tris-borate-EDTA buffer for 2 h at 150V. The gel is dried and exposed to XAR-5 film at −70 C overnight. The NF-HEV shifted DNA complexes are excised from the dried gel and incubated in 200 μl of 10 mM Tris-HCl, pH 8.0 for 3 h at 37 C. Ten microliters of the eluted DNA is used in a PCR to make probe for the next round of selection. PCR conditions are 10 nM Tris-HCl, pH 8.8, 50 mM KCl, 6 mM MgCl₂; 1 mM dithiothreitol; 0.18 μM primers P and R 10 μCi of [α-12P]dCTP; 50 μM each of dATP, dDTP and dGTP; and 20CM of dCTP. Final reaction volume is 100 μl, and the parameters are 20 cycles at 94° C. for 1 min, 62° C. for 1 min and 72° C. for 1 min. In subsequent rounds, 1.5 nM protein is used. After five rounds of selection by NF-HEV, pools of amplified oligonucleotides are digested with BamII and EcoRI and cloned into Bluescript KS- (Stratagene). The blue and white colony selection method is used to identify possible recombinants, and the composition of the insert is determined by dideoxy sequencing of the denatured single-stranded templates.

### Example 12

### High Throughput In Vitro Assay to Identify Inhibitors of NF-HEV Polypeptide or NF-HEV Interactions with Nonspecific DNA Targets

High throughput assays for the detection and quantification of NF-HEV-nonspecific DNA binding is carried out using a scintillation proximity assay. Materials are available from Amersham (Piscataway, N.J.) and assays can be carried out according to Gal S. et al, 6^{th} Ann. Conf. Soc. Biomol. Screening, 6-9 Sep. 2000, Vancouver, B.C.).

Random double stranded DNA probes are prepared and labeled using [³H]TTP and terminal transferase to a suitable specific activity (e.g. approx. 420 i/mmol). NF-HEV protein or a portion thereof is prepared and the quantity of NF-HEV protein or a portion thereof is determined via ELISA. For assay development purposes, electrophoretic mobility shift assays (EMSA) can be carried out to select suitable assay parameters. For the high throughput assay, ³H labeled DNA, anti-NF-HEV monoclonal antibody and NF-HEV in binding buffer (Hepes, pH7.5; EDTA; DTT; 10 mM ammonium sulfate; KCl and Tween-20) are combined. The assay is configured in a standard 96-well plate and incubated at room temperature for 5 to 30 minutes, followed by the addition of 0.5 to 2 mg of PVT protein A SPA beads in 50-100 μl binding buffer. The radioactivity bound to the SPA beads is measured using a TopCount™ Microplate Counter (Packard Biosciences, Meriden, Conn.).

### Example 13

### High Throughout In Vitro Assay to Identify Inhibitors of NF-HEV Polypeptide or NF-HEV Interactions with Specific DNA Targets

High throughput assays for the detection and quantification of NF-HEV specific DNA binding is carried out using a scintillation proximity assay. Materials are available from Amersham (Piscataway, N.J.) and assays can be carried out according to Gal S. et al, 6 h Ann. Conf. Soc. Biomol. Screening, 6-9 Sep. 2000, Vancouver, B.C.).

NF-HEV-specific double stranded DNA probes corresponding to NF-HEV DNA binding sequences obtained according to Example 11 are prepared. The probes are labeled using [³H]TTP and terminal transferase to a suitable specific activity (e.g. approx. 420 i/mmol). NF-HEV protein or a portion thereof is prepared and the quantity of NF-HEV protein or a portion thereof is determined via ELISA. For assay development purposes, electrophoretic mobility shift assays (EMSA) can be carried out to select suitable assay parameters. For the high throughput assay, ³H labeled DNA, anti-NF-HEV monoclonal antibody, lIg non-specific DNA (double or single stranded poly-dAdT) and NF-HEV protein or a portion thereof in binding buffer (Hepes, pH7.5; EDTA; DTT; 10 mM ammonium sulfate; KCl and Tween-20) are combined. The assay is configured in a standard 96-well plate and incubated at room temperature for 5 to 30 minutes, followed by the addition of 0.5 to 2 mg of PVT protein A SPA beads in 50-100 μl binding buffer. The radioactivity bound to the SPA beads is measured using a TopCount™ Microplate Counter (Packard Biosciences, Meriden, Conn.).

### Example 14

Preparation of Antibody Compositions

Substantially pure NF-HEV protein or a portion thereof is obtained. The concentration of protein in the final preparation is adjusted, for example, by concentration on an Amicon filter device, to the level of a few micrograms per ml. Monoclonal or polyclonal antibodies to the protein can then be prepared as follows: Monoclonal Antibody Production by Hybridoma Fusion Monoclonal antibody to epitopes in the NF-HEV protein or a portion thereof can be prepared from murine hybridomas according to the classical method of Kohler and Milstein (Nature, 256: 495, 1975) or derivative methods thereof (see Harlow and Lane, Antibodies A Laboratory Manual, Cold Spring Harbor Laboratory, pp. 53-242, 1988).

Briefly, a mouse is repetitively inoculated with a few micrograms of the NF-HEV protein or a portion thereof over a period of a few weeks. The mouse is then sacrificed, and the antibody producing cells of the spleen isolated. The spleen cells are fused by means of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall, E., Meth. Enzymol. 70: 419 (1980). Selected positive clones can be expanded and their monoclonal antibody product harvested for use. Detailed procedures for monoclonal antibody production are described in Davis, L. et al. Basic Methods in Molecular Biology Elsevier, New York. Section 21-2.

Polyclonal Antibody Production by Immunization

Polyclonal antiserum containing antibodies to heterogeneous epitopes in the NF-HEV protein or a portion thereof can be prepared by immunizing suitable non-human animal with the NF-HEV protein or a portion thereof, which can be unmodified or modified to enhance immunogenicity. A suitable nonhuman animal, preferably a non-human mammal, is selected. For example, the animal may be a mouse, rat, rabbit, goat, or horse. Alternatively, a crude protein preparation which, has been enriched for NF-HEV or a portion thereof can be used to generate antibodies. Such proteins, fragments or preparations are introduced into the non-human mammal in the presence of an appropriate adjuvant (e.g. aluminum hydroxide, RIBI, etc.) which is known in the art. In addition the protein, fragment or preparation can be pretreated with an agent which will increase antigenicity, such agents are known in the art and include, for example, methylated bovine serum albumin (mBSA), bovine serum albumin (BSA), Hepatitis B surface antigen, and keyhole limpet hemocyanin (KLH). Serum from the immunized animal is collected, treated and tested according to known procedures. If the serum contains polyclonal antibodies to undesired epitopes, the polyclonal antibodies can be purified by immunoaffinity chromatography.

Effective polyclonal antibody production is affected by many factors related both to the antigen and the host species. Also, host animals vary in response to site of inoculations and dose, with both inadequate or excessive doses of antigen resulting in low titer antisera. Small doses (ng level) of antigen administered at multiple intradermal sites appears to be most reliable. Techniques for producing and processing polyclonal antisera are known in the art, see for example, Mayer and Walker (1987). An effective immunization protocol for rabbits can be found in Vaitukaitis, J. et al. J. Clin. Endocrinol. Metab. 33: 988-991 (1971). Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony, O. et al., Chap. 19 in: Handbook of Experimental Immunology D. Wier (ed) Blackwell (1973). Plateau concentration of antibody is usually in the range of 0.1 to 0.2 mg/ml of serum (about 12: M). Affinity of the antisera for the antigen is determined by preparing competitive binding curves, as described, for example, by Fisher, D., Chap. 42 in: Manual of Clinical Immunology, 2d Ed. (Rose and Friedman, Eds.) Amer. Soc. For Microbiol., Washington, D.C. (1980).

Antibody preparations prepared according to either the monoclonal or the polyclonal protocol are useful in quantitative immunoassays which determine concentrations of antigen-bearing substances in biological samples; or they are also used semi-quantitatively or qualitatively to identify the presence of antigen in a biological sample. The antibodies may also be used in therapeutic compositions for killing cells expressing the protein or reducing the levels of the protein in the body.

### Example 15

### In Situ Hybridization Analysis of NF-HEV mRNA Expression in Crohn's Disease and Rheumatoid Arthritis

In situ hybridization (ISH) analysis of NF-HEV mRNA expression in Crohn's disease and rheumatoid arthritis was performed following the non-radioactive ISH method originally described by B. St Croix et al. (Science 18, no 289, 1197-202, 2000) with minor modifications. Briefly, antisense and sense NF-HEV riboprobes were generated by PCR by incorporating the T7 promoter sequence (5′-GGATCCTAATACGACTCACTATAGGGAGA-3′) (SEQ ID NO: 20) into the forward or reverse NF-HEV primer. The PCR were performed on a plasmid template containing the complete coding sequence of NF-HEV (pcDNA3.1-NF-HEV) using conventional procedures. PCR amplification were controlled on a gel electrophoresis before performing in vitro transcriptions with Dig-labeled UTP on 200 ng of PCR template with the DIG RNA labeling mix (Roche) following manufacturer instructions. RNA integrity and concentration were verified by running 5 μl of RNA on a 6% TBE-urea gel along side known concentrations of marker (RNA century-Plus size markers, Ambion). Frozen sections from biopsies of patients with RA and Crohn were prepared according to conventional histological procedures skilled in the art. After fixation in 4% paraformaldehyde/PBS 1 hour, sections were rinsed twice in PBS for 5 min on ice and then incubated with 0.2 N HCl, 5 min at RT to inactivate endogenous alkaline phosphatase activity. Sections were immediately after incubated in 2% Dako's ready to use Pepsin for 5 mins at 37° C. before rinsing for 5 min on ice with PBS. Slides were then acetylated in 0.1 M Triethylamine (pH 8.0) for 5 mins, rinsed for 5 mins in PBS and equilibrated for 5 to 10 mins in 5×SSC. Slides were then removed from SSC and a CoverWell incubation chamber gasket (Molecular Probes cat#18156) were placed around the section for the prehybridization step. Briefly, 150 μl of mRNA hybridization buffer (Dako cat#S3304) were added to the chamber and allowed to incubate 1 hour at 55° C. In the meantime, digoxigenin-labeled riboprobe were added in a 1.5 ml RNAse-free microfuge tube to a final concentration of 200 ng/ml, denatured at 95° C. for 3 mins and chilled immediately on ice. Hybridization step were performed by adding 150>11 of denaturated labeled riboprobes in the chamber. After sealing the chamber with a covers lip, slides were placed in a humid box and incubated overnight at 55° C. A day after, slides were rinsed by incubating in 50 ml tubes containing 30 ml 2×SSC for 5 min in a 45° C. water bath, then rinsed twice in TNE buffer at 45° C. for 5 mins. Excess unhybridized riboprobe were removed by incubating 250 ul RNAse A/T1 cocktail (Ambion cat# 2288) diluted 1:35 in TNE buffer at 37° C. for 1 hour. Slides were then stringently wash twice with 30 ml 2×SSC, 50% deionized formamide for 20 min at 55° C. and then rinsed once with 30 ml 0.08×SSC for a further 20 mins at 55° C.

The following steps use conventional procedures and reagents classically used for the detection of non-radioactive nucleic acids using the biotin-tyramide amplification cycle system (Dako). Briefly, slides were rinsed with 1×TBST buffer for 3 min at RT and incubated with 150 μl of blocking buffer containing 1:20 dilution of rabbit immunoglobulin fraction (Dako cat#X0903) for 30 min at RT. Sections were then incubated with HRP-anti-DIG (Dako cat#P5104) diluted 1:150 in blocking buffer for 45 min at RT, washed three times for 4 ins with 1×TBST buffer before adding directly one drop of ready-to-use bitingly-tyramide (Dako gunpoint Kit) and further incubated in dark for 8 min at RT. Slides were the rinsed three times for 4 ins with 1×TBST buffer and incubated with rabbit HRP-anti-biotin (Dako) diluted 1:150 in blocking buffer for 20 min at RT, rinsed again as above before adding again one drop of ready-to-use bitingly-tyramide (Dako gunpoint Kit) and further incubated in dark for 5 min at RT. After washing three times for 4 ins with 1×TBST buffer at RT, slides were incubated with rabbit AP-anti-biotin (Dako) diluted 1:75 with blocking buffer for 20 min at RT in dark. After a last washing step, specific signal detection was performed by incubating the AP-substrate (Fast Red tablets, Sigma) for 20 min at RT in dark The signal was carefully monitored under a fluorescent microscope (Leach) until the apparition of specific fluorescence signal after comparison with the background observed on the control slide. Reactions were stopped by incubating slides in water for 3 min at RT. Sections were allowed to dry from 5 min at RT and mounted after adding one drop of Supermount permanent aqueous mounting media (Biogenex). An extra immunostaining step was performed for some slides and followed conventional immunostaining procedures to get dual-color labelled pictures.

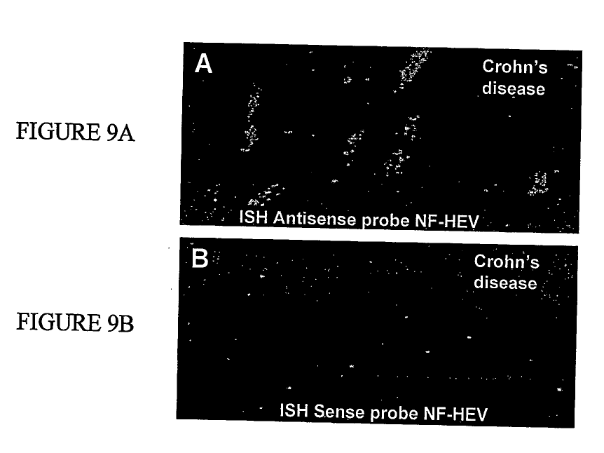

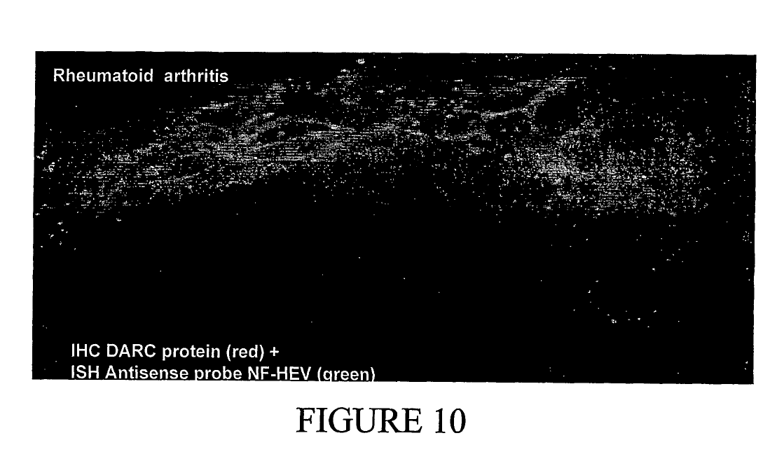

These ISH analyses revealed specific expression of NF-HEV mRNA in endothelial cells from small blood vessels associated with Crohn's disease (FIG. 9A). As expected, no signal was observed using the sense control probe (FIG. 9B). Similar results were observed from small blood vessels associated with rheumatoid arthritis (FIG. 10).

### Example 16

### Upregulation of NF-HEV Expression in Primary Human Endothelial Cells by Pro-Inflammatory Cytokines TNFα, IL1β and IFNγ

The effects of pro-inflammatory cytokines on NF-HEV mRNA expression in primary human endothelial cells were analyzed using real time quantitative RT-PCR.

Stimulations assays: Trypsinized HUVECs, PMECs and HEVECs (36.000 cells per 12-wells plate) were plated one day before the stimulation and grown until sub-confluence. Complete medium was replaced by freshly prepared medium including cytokines and cells were stimulated for 16 hours at 37° C. Cytokine concentrations were chosen that have been shown to upregulate different adhesion molecules and being non-lethal for the cells as described by M. Raab et al. (Raab et al. (2002) Clin Chim Acta., 321:11-16). The following final concentrations were used: IFNγ (10 ng/ml), TNFβ (2.5 ng/ml), IL-113 (10 ng/ml), IFNγ+TNFα (10 and 2.5 ng respectively), IFNγ+IL-1β (10+10 ng/ml respectively), TNFα+IL-1β (2.5+10 ng/ml respectively), and LTα1β2 (100 ng/ml). After 16 hours of stimulation, cells were washed in PBS and lyzed in the plates in RLT buffer (RNeasy kit, QIAgen) and immediately proceeded to RNA isolation.

Quantitative RT-PCR: Two micrograms total RNA were reverse transcribed using Oligo(dT) and Superscript II enzyme in a 20 μl reaction. Specific mRNA transcripts were quantified by real-time PCR using the Light Cycler (Roche Diagnostics). Primers were designed using the LC Probe Design program (Roche Diagnostics). Equal amounts of RNA-input were amplified using the QuantiTect SYBR Green PCR kit from Qiagen according to the manufacturer's protocol. The amplification coefficient (K_{gene}) of NF-HEV and GAPDH was calculated from serial dilutions of cDNA (Table 1). For quantifying the copy numbers of NF-HEV, a plasmid containing the complete coding sequence of NF-HEV (pcDNA3.1-NFHEV) was linearized by BamHI digestion and the number of NF-HEV molecules per μl was calculated using the following equation:

$$\frac{gram \text{/} µl}{bp \times 660} \times 6.022 \times {10}^{23}$$

TABLE 1

| | | Product size | Amplification | |
|---|---|---|---|---|
| Gene (human) | Primer sequence (5′-3′) | (bp) | Coefficient | SEQ ID NO: |
| NF-HEV.fwd | AAATGAATCAGGTGACGG | 130 | 1.87 | 21 |
| NF-HEV.rev | TGGCAGTGGTTTTTCACA | | | 22 |
| IL-8.fwd | AGTGATTGAGAGTGGGACC | 136 | 2.25 | 23 |
| IL-8.rev | ATTCTCAGCCCTCTTCAA | | | 24 |
| GAFDH.fwd | AAATCCCATCACCATCTTCC | | 2.05 | 25 |
| GAPDH.rev | CATGAGTCCTTCCACGATACC | | | 26 |
| ICAM-1.fwd | GCTCTGTTCCCAGGAC | 187 | n.e. | 27 |
| ICAM-1.rev | GTTCATACACCTTCCGGTTG | | | 28 |
| CXCL1/2.fwd | AGTGTGAAGGTGAAGTCC | 154 | n.e. | 29 |
| CXCL1.rev | GGTCAGTTGGATTTGT | | | 30 |
| CXCL2.rev | GGTCAGTTGGATTTGC | 154 | n.e. | 31 |
| CXCL6.fwd | TGCTGCTCCTGCTGAC | 175 | n.e. | 32 |
| CXCL6.rev | GCTACCACTTCCACCT | | | 33 |
| CCL2.fwd | CCAGATGCAATCAATGCC | 180 | n.e. | 34 |
| CCL2.rev | CTGAACCCACTTCTGCT | | | 35 |

n.e. = not evaluated

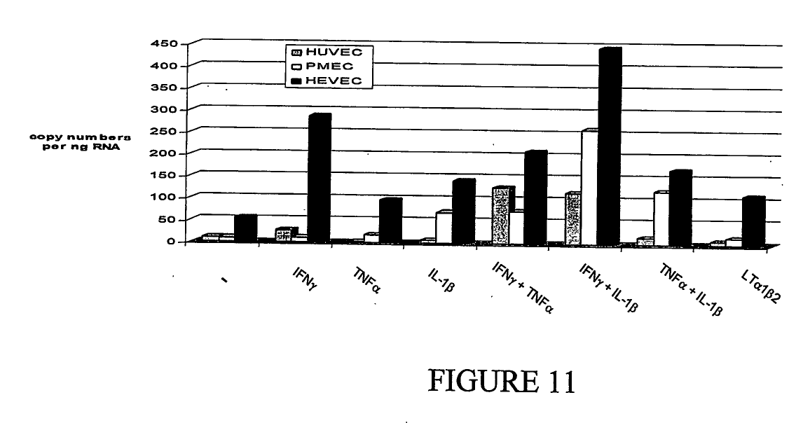

The above-described quantitative RT-PCR analyses revealed that combination of pro-inflammatory cytokines such as IFNγ, TNFα, and IL-1β, results in strong induction of NF-HEV mRNA expression in all types of primary human endothelial cells that were analyzed (HUVEC, PMEC, HEVEC) (FIG. 11).

### Example 17

### Retrovirus Mediated Expression of NF-HEV in Primary Human Endothelial Cells

The method described below uses retroviral derived vectors to transduce human primary umbilical vein endothelial cells (HUVEC) with the NF-HEV gene.

Construction of retroviral vectors and generation of retroviral supernatants: The complete coding region of human NF-HEV was amplified by nested reverse transcription polymerase chain reaction (RT-PCR) amplification using human specific primers for NF-HEV (NF-HEV.fwd 5′-GAATACTGAAAAATGAAGCC-3′ (SEQ ID NO: 36), NF.HEV.rev 5′-GACTCACAGGTTTCCATCAAC-3′ (SEQ ID NO: 37)). PCR products were cloned into the Moloney murine leukemia virus-based retroviral vector pLZRS (Kinsella and Nolan (1996) Hum Gene Ther. 7:1405-1413) which were kindly provided by MHC Heemskerk (Leiden University Medical Center, Leiden, The Netherlands) and originally described by G. Nolan (Stanford University, Palo Alto, Calif.). This bicistronic retroviral vector encodes a non-signaling, truncated form of the nerve growth factor receptor (ΔNGF-R) and enables positive immuno-selection of transduced cells. NF-HEV containing plasmids and empty vectors were transfected into φ-NX-A cells using calcium phosphate (Invitrogen, Merelbeke, Belgium). From day 2, cells were cultured in Iscoves modified Dulbecco medium supplemented with 8% fetal calf serum (FCS), 4 mM glutamine, 20 U/ml streptomycin, 20 U/mil penicillin, 50 ng/ml amphotericin-B, 25 mM HEPES and 2 mM puromycin (Sigma-Aldrich, Oslo, Norway). Two weeks after transfection, cells were plated into 10-cm Petri dishes in 10 ml IMDM-medium without puromycin. After 24 hours, medium was refreshed, and the next day retroviral supernatants were harvested and frozen at −70° C.

Retroviral transduction of human umbilical vein endothelial cells (HUVECs): Exponentially growing HUVECs were transduced with retroviral supernatants based on the method originally described by Zheng et al. (Zheng et al. (2000) J Immunol., 164:4665-4671) with minor modifications. Briefly, 1 ml retroviral supernatants were preincubated with 10 mg/ml DOTAP N-[1-(−2,3-dioleoyloxy)propyl]-N,N,N-trimethylammonium methylsulfate (Roche, Indianapolis, Ind.) on ice for 10 minutes. Trypsinized HUVECS (5×10⁵) were resuspended in the virus-DOTAP mixture and plated in 24-well plates. After 4-6 hours at 37° C., retroviral supernatants were removed and replaced by MCDB 131 medium containing 7.5% FCS, 10 ng/nl EGF, 1 ng/ml bFGF, 1 μg/ml hydrocortisone, 50 μg/ml gentamicin, and 250 ng/ml fungizone. A second, third and fourth transduction of adherent cells was performed by a 4-6 hours incubation with virus-DOTAP mixtures performed on day 1, 6 and 9 respectively. Ten days after the last transduction, NGPR-positive cells were positively selected by first incubating with a murine anti-human NGF-R monoclonal antibody 20.4 (ATCC) for 15 minutes at 4° C. and subsequently with rat anti-mouse IgG1 MACS-beads for 15 minutes at 4° C. After two rounds of MACS-beads selection, >99% NGFR positive cells were obtained in both empty vector- and NF-HEV transduced HUVECs.

### Example 18

### Identification of NF-HEV Target Genes by DNA Microarrays and Real-Time Polymerase Chain Reaction (PCR)

To better understand the function of NF-HEV as a nuclear factor in the vasculature, we globally profiled NF-HEV target genes in primary human endothelial cells using retroviral gene transfer and Affymetrix oligonucleotide-based microarray technology. We quantitated the NF-HEV mediated changes in expression of 33 000 well substantiated human genes by transducing human vascular endothelial cells with either empty vector (HVVEC+NGFR) or NF-HEV expression vector (EC+NP-HEV+NGFR). Over-expression of NP-HEV in HUVECs was verified both at RNA and protein levels with standard quantitative PCR and immunostaining procedures (see previous Examples).

RNA isolation and chipanalysis: Total RNA isolation and on-column DNase treatment was performed using the RNeasy Mini Kit according to instructions of the manufacturer (Qiagen GmbH, Hilden, Germany). Chip analysis experiments were performed in the lab of Kari Alitalo, Helsinki, Finland. The RNA quality was determined by Northern blot analysis. In vitro transcription and biotin labeling was performed according to Affymetrix guidelines. Biotinylated cRNA was hybridized to the human U133A chip containing 33,000 well-substantiated human genes. Differentially expressed genes were analyzed by using the MicroSuite 5.0 algorithms provided by Affymetrix. Differential expression was verified by quantitative PCR using the Light Cycler (Roche Diagnostics).

Real-Time Quantitiative Polymerase Chain Reaction (Q-PCR): Two micrograms total RNA was reverse transcribed using Oligo(dT) and Superscript II enzyme in a 20 μl reaction. Specific mRNA transcripts were quantified by real-time PCR using the Light Cycler (Roche Diagnostics) and cDNAs synthesized from RNA isolated from HUVEC cells infected with NF-HEV or control retroviral constructs. Primers were designed using the LC Probe Design program (Roche Diagnostics). Equal amounts of RNA-input were amplified using the QuantiTect SYBR Green PCR kit from Qiagen according to the manufacturer's protocol. The amplification coefficient (K_{gene}) of NF-HEV, IL-8 and GAPDH was calculated from serial dilutions of cDNA (Table 1). Quantitative PCR for the other genes identified was performed as described for NF-HEV (Table 1). Fold inductions compared to non-stimulated cells (Table 2) were calculated by using the following equation: K_{gene}^{ΔCp}, where ΔCₚ is (the crossing point for the RT-PCR from unstimulated cells)−(the crossing point for the RT-PCR from stimulated cells).

Together, the DNA microarray and Q-PCR analyses revealed that NF-HEV modulates expression of pro-inflammatory chemokines CXCL1/GROα, CXCL2/GROβ, CXCL6, CXCL8/IL8, CCL2/MCP1 and cell adhesion molecule ICAM1 (Table 2).

TABLE 2

| | | | | | | | | Fold change RT- |
|---|---|---|---|---|---|---|---|---|
| | | Detection p- | | | Change p- | | Fold change Real- | PCR second |
| Signal | Detection | value | fold change | Change | value | | time PCR | transduction |
| 436.3 | P | 0.000244 | 8.6 | I | 0.000020 | CXCL1 (GRO-alpha) chemokine | 15.8 | 73.5 |
| 65.3 | P | 0.001953 | 7.0 | I | 0.000068 | CXCL6 (GCP-2) chemokine | 142.0 | 68.6 |
| 756.7 | P | 0.001221 | 6.5 | I | 0.000052 | interleukin 8 | 10.1 | 21.0 |
| 105.9 | P | 0.000244 | 5.7 | I | 0.000020 | CCL2 (MCP-1) chemokine | 3.0 | 5.3 |
| 179.9 | P | 0.000244 | 5.3 | I | 0.000060 | CXCL2 (GRO-beta) chemokine | 5.5 | 17.6 |
| 111.8 | P | 0.002930 | 2.1 | I | 0.000346 | ICAM-1 (CD54) | 1.7 | 5.9 |
| | | | | | | NF-HEV | 1595.7 | 3902.0 |

CXCL1 (GRO-alpha) chemokine: (Amino Acid SEQ ID NO: 38; Nucleic Acid SEQ ID NO: 39);
CXCL6 (GCP-2) chemokine: (Amino Acid SEQ ID NO: 40; Nucleic Acid SEQ ID NO: 41);
interleukin 8: (Amino Acid SEQ ID NO: 42; Nucleic Acid SEQ ID NO: 43);
CCL2 (MCP-1) chemokine: (Amino Acid SEQ ID NO: 44; Nucleic Acid SEQ ID NO: 45);
CXCL2 (GRO-beta) chemokine: (Amino Acid SEQ ID NO: 46; Nucleic Acid SEQ ID NO: 47);
ICAM-1 (CD54): (Amino Acid SEQ ID NO: 48; Nucleic Acid SEQ ID NO: 49)

### Example 19

### NF-HEV Induces Expression of Pro-Inflammatory Chemokines CCL2/MCP1, CXCL1/GROα, and CXCL8/IL8

To confirm the microarray and Q-PCR data (Table 2), we analyzed the capacity of NF-HEV to induce chemokine expression at the protein level. Two types of assays were performed: ELISA assays and immunofluorescence stainings.

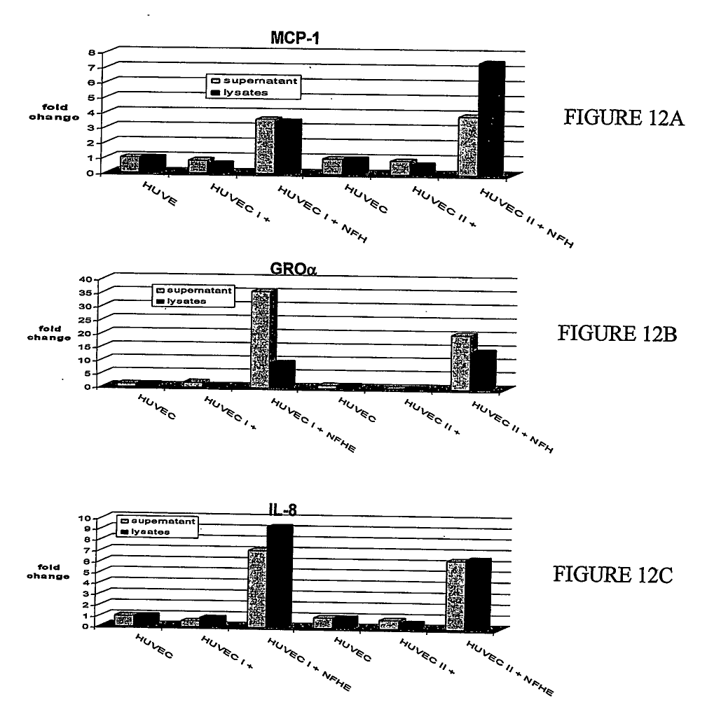

ELISA assay: To quantify the amounts of GROα, MCP-1 and IL-8 protein, HUVECs were seeded out in confluence (1.6×10⁴ cells per 96-well trays in triplicate) and cultivated for 4 days. The medium was refreshed every day to maintain good culture conditions. One hour before harvesting supernatants, medium was refreshed. After harvesting supernatants, cells were washed three times in pre-heated PBS and lysed in 50 μl 1% NP-40 in 50 mM Tris-HCl and 150 mM NaCl containing a mixture of protease inhibitors. Cell supernatants and lysates from individual wells were analyzed for IL-8, GROa and MCP-1 protein by ELISA techniques (FIG. 12A-C). The chemokines were measured using matched antibody pairs from R&D systems according to the recommendations of the manufacturer with minor modifications. Microtiter plates were incubated overnight with coating antibody diluted in PBS at RT, washed in water and blocked by 1% (w/v) BSA in PBS for 2 hours at RT. Before adding the samples, plates were washed 4 times in PBS, 0.05% Tween 20. Samples (50 μl/well) were incubated overnight followed by detection antibody for 1.5 hour, and subsequently alkaline phosphatase-conjugated streptavidin for 1.5 hour at RT. P-nitrophenyl phosphate in diethhanolamine buffer was developed for 10-60 minutes and the absorbance was measured at 405 nm with a Tecan Sunrise Microplate Reader (Tecan Austria Gesellschaft, Salzburg, Austria). Standard curves were generated form 3-fold dilutions of recombinant proteins (R&D systems).

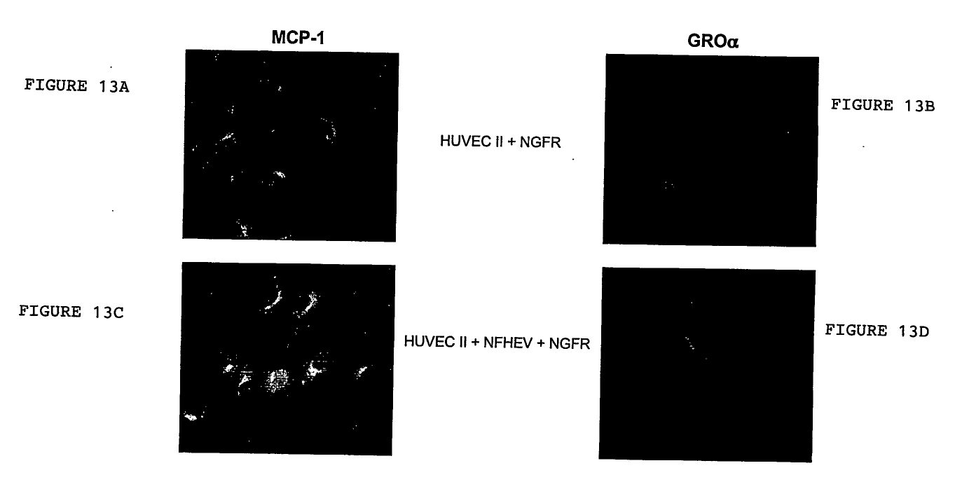

Immunofluorescence staining: Expression of chemokines CCL2/MCP1 and CXCL1/GROα was analyzed by immunostaining of cells grown on Lab-Tek chamber slides (Ninc, Roskilde, Denmark) coated with 1% (w/v) gelatin type A form porcine. Monolayers of NF-HEV transduced HUVECs and empty vector-transduced cells were fixed in 4%-PFA (15 minutes at RT), permeabilized by incubating 30 minutes in 0.1% saponin at RT and microwave treated in citrate buffer pH 6.0 for 3 minutes at 900 Watt followed by 30 minutes at 90 Watt. Cells were subsequently incubated with affinity purified rabbit anti human GROα and MCP-1 chemokine antibodies (Peprotech, 500-P92 and 500-P34 respectively) overnight at 4° C., followed by biotin-labeled goat anti-rabbit antibody for 3 hours at RT and Cy3-conjugated streptavidin for 1 hour at RT. Analysis by fluorescence microscopy revealed upregulation of chemokines CCL2/MCP1 and CXCL1/GROα in NF-HEV tranduced cells (HUVEC II+NF-HEV+NGFR) (FIG. 13C-D), compared to cells transduced with the control retrovirus vector (HUVEC II+NGFR) (FIG. 13A-B).

### Example 20

Reduction of NF-HEV Gene Expression by siRNA Reduces Expression of Pro-Inflammatory Chemokines

This experiment is designed to demonstrate that a small-interfering RNA (siRNA) specific to a portion of the coding nucleotide sequence for NF-HEV (SEQ ID NO: 1) can reduce the expression of the NF-HEV polypeptide and thereby reduce the amount of pro-inflammatory chemokines expressed by HEVEC cells. siRNA duplexes composed of 21-nucleotide sense and antisense strands are synthesized. The RNA oligonucleotides are specific to one or more discrete or overlapping 21 consecutive base pair portions of the coding region of SEQ ID NO: 1. HEVEC cells are plated in 6 cm wells at 2.5×10⁵ cells per well 24 h before transfection. Twenty micromolar siRNA in 25 μl of Oligofectamine reagent (Invitrogen) is incubated in medium for 20 min, then the transfection mixture is added to cells, incubated at 37° C. for 4 h, followed by addition of fresh medium. At 36 hours after transfection, cells are analyzed for expression of a NF-HEV transcript and levels of pro-inflammatory chemolines.

### Example 21

### Reduction of NF-HEV Gene Expression by siRNA Reduces Inflammation in a Mouse Model for Rheumatoid Arthritis

Experiments to demonstrate that NF-HEV-specific siRNA functions to reduce inflammation are performed using a mouse model for rheumatoid arthritis, the well-known collagen-induced arthritis model. In each experiment, male DBA/1 mice are immunized with collagen on day 21 and are boosted on day 0. Mice are treated daily from days 0-14 with IP injections of siRNA specific to one or more discrete or overlapping 21 consecutive base pair portions of the coding region of SEQ ID NO: 1, and compared to mice treated with control siRNA (n=15/group in each experiment). The incidence and severity of arthritis is monitored in a blind study. Each paw is assigned a score from 0 to 4 as follows: 0=normal; 1=swelling in 1 to 3 digits; 2=mild swelling in ankles, forepaws, or more than 3 digits; 3=moderate swelling in multiple joints; 4=severe swelling with loss of function. Each paw is totaled for a cumulative score/mouse. The cumulative scores are then totaled for mice in each group for a mean clinical score. Groups of 15 mice are treated with the indicated doses of NF-HEV-specific siRNA or with 150 μg/day of nonspecific control siRNA. The capacity of NF-HEV-specific siRNA to reduce the disease incidence and severity of arthritis is determined by comparison with the control group.

Similar siRNA experiments are performed in mice to demonstrate that NF-HEV-specific siRNA functions to reduce inflammation when delivered using adenovirus vectors. In particular, adenovirus vectors are designed to deliver nucleic acids encoding NF-HEV-specific siRNAs to inflamed tissue in a mouse model for rheumatoid arthritis.

Adenovirus expression vectors comprising nucleic acids encoding one or more discrete or overlapping 21 consecutive base pair portions of the coding region of SEQ ID NO: 1 are prepared. Nucleic acid constructs which are capable of forming a double-stranded siRNA and which are also appropriate for cloning into an adenovirus expression vector are, for example, nucleic acids having a 21-base pair inverted repeat separated by about five nucleotides. Upon transcription of the inverted repeat region forms a self-complementary dsRNA having an approximately 5 base single-stranded hairpin region.

Male DBA/1 mice are prepared as described above. For viral dosing of mice, the DBA/1 mice are administered recombinant adenoviruses comprising one or more discrete or NF-HEV-specific constructs as described above via tail vein injection using a 0.5 ml tuberculin syringe at doses of 0.6-1.2×10¹¹ viral particles/animal. Four groups of animals (n=5-15/group) are treated with either adenovirus comprising nucleic acid encoding NF-HEV-specific siRNA, control adenovirus comprising nucleic acid encoding nonspecific RNA or buffer only.

The incidence and severity of arthritis is monitored in a blind study. Each paw is assigned a score from 0 to 4 as follows: O=normal; 1=swelling in 1 to 3 digits; 2=mild swelling in ankles, forepaws, or more than 3 digits; 3=moderate swelling in multiple joints; 4=severe swelling with loss of function. Each paw is totaled for a cumulative score/mouse. The cumulative scores are then totaled for mice in each group for a mean clinical score. The capacity for NF-HEV-specific siRNA to reduce the disease incidence and severity of arthritis is determined by comparison of the treatment groups to the control groups.

It will be appreciated by one of ordinary skill in the art that expression of a NF-HEV-specific siRNA can be used to ameliorate the symptoms associated with any NF-HEV-mediated condition. In some embodiments such expression can be the result of gene therapy.

### Example 22

### Reduction of NF-HEV Gene Expression by an Antisense Nucleic Acid Reduces Expression of Pro-Inflammatory Chemokines

This experiment is designed to demonstrate that an antisense nucleic acid specific to a portion of the coding nucleotide sequence for NF-HEV (SEQ ID NO: 1) can reduce the expression of the NF-HEV polypeptide and thereby reduce the amount of pro-inflammatory chemokines expressed by HEVEC cells. Single-stranded antisense nucleic acids, antisense analogs having phosphorothioate backbones or chiral phosphorothioate backbones and PNA antisense analogs complementary to the NF-HEV sense strand (SEQ ID NO: 1) are constructed. These antisense nucleic acids and antisense analogs correspond to discrete or overlapping 20, 25, 30, 35, 40, 45, 50, 75, 100, 150 and 200 consecutive base pair portions of the sequence complementary to the coding region of SEQ ID NO: 1. HEVEC cells are plated in 6 cm wells at 2.5×10⁵ cells per well 24 h before transfection. Twenty micromolar antisense nucleic acid or antisense analog in 25 μl of Oligofectamine reagent (Invitrogen) is incubated in medium for 20 min, then the transfection mixture is added to cells, incubated at 37° C. for 4 h, followed by addition of fresh medium. At 36 hours after transfection, cells are analyzed for expression of a NF-HEV transcript and levels of pro-inflammatory chemokines.

### Example 23

### Reduction of NF-HEV Gene Expression by an Antisense Nucleic Acid Reduces Inflammation in a Mouse Model for Rheumatoid Arthritis

Experiments to demonstrate that NF-HEV-specific antisense nucleic acids function to reduce inflammation are performed using a mouse model for rheumatoid arthritis, the well-known collagen-induced arthritis model. The experiments are performed as in Example 21 except the siRNA constructs are substituted with the antisense constructs, and where appropriate, the antisense analog constructs of Example 22.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit and scope of that which is described and claimed.

As used in the claims below and throughout this disclosure, by the phrase “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

1-22. (canceled)

23. A method of ameliorating symptoms of a condition associated with inflammation, said method comprising:
identifying a subject having symptoms of a condition associated with inflammation; and
modulating in said subject the level or activity of the NF-HEV polypeptide or a biologically active fragment thereof, thereby ameliorating symptoms of a condition associated with inflammation.

24. The method of claim 23, wherein the level or activity of said NF-HEV polypeptide or a biologically active fragment thereof is modulated by altering the expression of a nucleic acid encoding said NF-HEV polypeptide or a biologically active fragment thereof.

25. The method of claim 23, wherein the level or activity of said NF-HEV polypeptide or a biologically active fragment thereof is modulated by administering a compound to said subject.

26. The method of claim 23, wherein modulating the level or activity of said NF-HEV polypeptide or a biologically active fragment thereof modulates the level or activity of a pro-inflammatory chemokine.

27. (canceled)

28. The method of claim 26, wherein the level or activity of said pro-inflammatory chemokine is reduced.

29. The method of claim 23, wherein the level or activity of said NF-HEV polypeptide or a biologically active fragment thereof is reduced.

30. The method of claim 29, wherein the level or activity of said NF-HEV polypeptide or a biologically active fragment thereof is reduced by reducing the expression of a nucleic acid encoding said NF-HEV polypeptide or a biologically active fragment thereof.

31. The method of claim 30, wherein the expression of a nucleic acid encoding said NF-HEV polypeptide or a biologically active fragment thereof is reduced by providing an antisense nucleic acid complementary to at least a portion of said NF-HEV polypeptide or a biologically active fragment thereof.

32. The method of claim 29, wherein the level or activity of said NF-HEV polypeptide or a biologically active fragment thereof is reduced by reducing the activity or level of a pro-inflammatory cytokine.

33. A method of ameliorating the symptoms of a condition associated with inflammation, said method comprising modulating the level of transcription of at least one promoter responsive to an NF-HEV polypeptide or biologically active fragment thereof.

34. The method of claim 33, wherein the level of transcription of said at least one promoter responsive to an NF-HEV polypeptide or biologically active fragment thereof is reduced.

35. The method of claim 33, wherein modulating the level or activity of said promoter modulates the level or activity of a pro-inflammatory chemokine.

37. The method of claim 35, wherein the level or activity of said pro-inflammatory chemokine is reduced.

38-57. (canceled)

58. A method of identifying a candidate inhibitor of an NF-HEV polypeptide, said method comprising:
a) contacting an NF-HEV polypeptide selected from the group consisting of: a biologically active NF-HEV polypeptide, a polypeptide having at least about 80% amino acid sequence identity to a biologically active NF-HEV polypeptide, and biologically active fragments of either of the foregoing with a test compound; and
b) determining whether said compound selectively binds to said polypeptide, wherein a determination that said compound selectively binds to said polypeptide indicates that said compound is a candidate inhibitor of said polypeptide.

59. The method of claim 58, wherein a determination that said compound selectively binds to said polypeptide indicates that said compound is a candidate compound for the treatment of a chronic inflammatory disorder.

60. A method of identifying a candidate inhibitor of an NF-HEV polypeptide selected from the group consisting of: a biologically active NF-HEV polypeptide, a polypeptide having at least about 80% amino acid sequence identity to a biologically active NF-HEV polypeptide, and biologically active fragments of either of the foregoing, said method comprising:
a) contacting said polypeptide with a test compound; and
b) determining whether said compound selectively inhibits at least one activity of said polypeptide, wherein a determination that said compound selectively inhibits at least one activity of said polypeptide indicates that said compound is a candidate inhibitor of said polypeptide.

61. The method of claim 60, wherein a determination that said compound selectively inhibits said at least one biological activity of said polypeptide indicates that said compound is a candidate compound for the treatment of a chronic inflammatory disorder.

62. A method of identifying a candidate NF-HEV inhibitor, said method comprising:
a) providing a cell comprising an NF-HEV polypeptide or a fragment comprising at least 6 consecutive amino acids thereof;
b) contacting said cell with a test compound; and
c) determining whether said compound selectively inhibits at least one NF-HEV activity, wherein a determination that said compound selectively inhibits activity of said polypeptide indicates that said compound is a candidate inhibitor of said polypeptide.

63. The method of claim 62, wherein a determination that said compound selectively inhibits said at least one biological activity of said polypeptide indicates that said compound is a candidate compound for the treatment of a chronic inflammatory disorder.

64. The method of claim 62, wherein step (a) comprises introducing a nucleic acid comprising the nucleotide sequence selected from the group consisting of: a biologically active NF-HEV polypeptide, a polypeptide having at least about 80% amino acid sequence identity to a biologically active NF-HEV polypeptide, and biologically active fragments of either of the foregoing; into said cell.

65. The method of claim 60, wherein said NF-HEV activity comprises modulating gene expression in an endothelial cell.

66. The method of claim 60, wherein said NF-HEV activity comprises modulating the inflammatory potential of an endothelial cell.

67. The method of claim 60, wherein said NF-HEV activity comprises modulating the phenotype of an endothelial cell.

68. The method of claim 60, wherein said NF-HEV activity comprises regulating HEV-like vessel development or maintenance.

69. The method of claim 60, wherein said NF-HEV activity comprises modulating the differentiation or proliferation of an endothelial cell.

70. The method of claim 60, wherein said NF-HEV polypeptide or fragment thereof comprises a homeodomain-like helix-turn-helix (HTH) DNA-binding domain.

71. The method of claim 58, wherein said NF-HEV polypeptide or fragment thereof consists essentially of the amino acid sequence of positions 61 to 78 of SEQ ID NO: 1 or 63 to 80 of SEQ ID NO: 2.

72-124. (canceled)

125. A method of identifying a candidate inhibitor of NF-HEV activity, said method comprising:
(a) providing an NF-HEV polypeptide of SEQ ID NOs: 4-5 or, a fragment comprising a contiguous span of at least 6 contiguous amino acids of a polypeptide according to SEQ ID NOs: 4-5;
(b) providing an NF-HEV target polypeptide or a fragment thereof; and
(c) determining whether a test compound selectively inhibits the ability of said NF-HEV polypeptide to bind to said NF-HEV target polypeptide, wherein a determination that said test compound selectively inhibits the ability of said NF-HEV polypeptide to bind to said NF-HEV target polypeptide indicates that said compound is a candidate inhibitor of NF-HEV activity.

126. (canceled)

127. The method of claim 23, wherein said NF-HEV polypeptide or biologically active fragment thereof comprises an amino acid sequence selected from the group consisting of amino acids 1-65 of SEQ ID NOs: 4-6.

128. The method of claim 33, wherein said NF-HEV polypeptide or biologically active fragment thereof comprises an amino acid sequence selected from the group consisting of amino acids 1-65 of SEQ ID NOs: 4-6.


SEQUENCE LISTING (49 sequences)

SEQ ID NO: 1 - DNA, 2645 bp, Homo sapiens
cacaagaatactgaaaaatgaagcctaaaatgaagtattcaaccaacaaaatttccacagcaaagtggaagaacacagcaagcaaagccttgtgtttcaagctgggaaaatcccaacagaaggccaaagaagtttgccccatgtactttatgaagctccgctctggccttatgataaaaaaggaggcctgttactttaggagagaaaccaccaaaaggccttcactgaaaacaggtagaaagcacaaaagacatctggtactcgctgcctgtcaacagcagtctactgtggagtgctttgcctttggtatatcaggggtccagaaatatactagagcacttcatgattcaagtatcacaggaatttcacctattacagagtatcttgcttctctaagcacatacaatgatcaatccattacttttgctttggaggatgaaagttatgagatatatgttgaagacttgaaaaaagatgaaaagaaagataaggtgttactgagttactatgagtctcaacacccctcaaatgaatcaggtgacggtgttgatggtaagatgttaatggtaaccctgagtcctacaaaagacttctggttgcatgccaacaacaaggaacactctgtggagctccataagtgtgaaaaaccactgccagaccaggccttctttgtccttcataatatgcactccaactgtgtttcatttgaatgcaagactgatcctggagtgtttataggtgtaaaggataatcatcttgctctgattaaagtagactcttctgagaatttgtgtactgaaaatatcttgtttaagctctctgaaacttagttgatggaaacctgtgagtcttgggttgagtacccaaatgctaccactggagaaggaatgagagataaagaaagagacaggtgacatctaagggaaatgaagagtgcttagcatgtgtggaatgttttccatattatgtataaaaatattttttctaatcctccagttattcttttatttccctctgtataactgcatcttcaatacaagtatcagtatattaaatagggtattggtaaagaaacggtcaacattctaaagagatacagtctgacctttacttttctctagtttcagtccagaaagaacttcatatttagagctaaggccactgaggaaagagccatagcttaagtctctatgtagacagggatccattttaaagagctacttagagaaataattttccacagttccaaacgataggctcaaacactagagctgctagtaaaaagaagaccagatgcttcacagaattatcattttttcaactggaataaaacaccaggtttgtttgtagatgtcttaggcaacactcagagcagatctcccttactgtcaggggatatggaacttcaaaggcccacatggcaagccaggtaacataaatgtgtgaaaaagtaaagataactaaaaaatttagaaaaataaatccagtatttgtaaagtgaataacttcatttctaattgtttaatttttaaaattctgatttttatatattgagtttaagcaaggcattcttacacgaggaagtgaagtaaattttagttcagacataaaatttcacttattaggaatatgtaacatgctaaaacttttttttttttaaagagtactgagtcacaacatgttttagagcatccaagtaccatataatccaactatcatggtaaggccagaaatcttctaacctaccagagcctagatgagacaccgaattaacattaaaatttcagtaactgactgtccctcatgtccatggcctaccatcccttctgaccctggcttccagggacctatgtcttttaatactcactgtcacattgggcaaagttgcttctaatccttatttcccatgtgcacaagtctttttgtattccagcttcctgataacactgcttactgtggaatattcatttgacatctgtctcttttcatttcttttaactaccatgcccttgatatatcttttgcacctgctgaacttcatttctgtatcacctgacctctggatgccaaaacgtttattctgctttgtctgttgtagaattttagataaagctattaatggcaatatttttttgctaaacgtttttgttttttactgtcactagggcaataaaatttatactcaaccatataataacattttttaactacataaaggagtagtttttattttaaagtcttagcaatttctattacaacttttcttagacttaacacttatgataaatgactaacatagtaacagaatctttatgaaatatgaccttttctgaaaatacatacttttacatttctactttattgagacctattagatgtaagtgctagtagaatataagataaaagaggctgagaattaccatacaagggtattacaactgtaaaacaatttatctttgtttcattgttctgtcaataattgttaccaaagagataaaaataaaagcagaatgtatatcatcccatctgaaaaacactaattattgacatgtgcatctgtacaataaacttaaaatgattattaaataatcaaatatatctactacattgtttatattattgaataaagtatattttccaaatgt

SEQ ID NO: 2 - DNA, 2486 bp, Mus musculus
aaacctgaaaaatgagacctagaatgaagtattccaactccaagatttccccggcaaagttcagcagcaccgcaggcgaagccctggtcccgccttgcaaaataagaagatcccaacagaagaccaaagaattctgccatgtctactgcatgagactccgttctggcctcaccataagaaaggagactagttattttaggaaagaacccacgaaaagatattcactaaaatcgggtaccaagcatgaagagaacttctctgcctatccacgggattctaggaagagatccttgcttggcagtatccaagcatttgctgcgtctgttgacacattgagcatccaaggaacttcacttttaacacagtctcctgcctccctgagtacatacaatgaccaatctgttagttttgttttggagaatggatgttatgtgatcaatgttgacgactctggaaaagaccaagagcaagaccaggtgctactacgctactatgagtctccctgtcctgcaagtcaatcaggcgacggtgtggatgggaagaagctgatggtgaacatgagtcccatcaaagacacagacatctggctgcatgccaacgacaaggactactccgtggagcttcaaaggggtgacgtctcgcctccggaacaggccttcttcgtccttcacaaaaagtcctcggactttgtttcatttgaatgcaagaatcttcctggcacttacataggagtaaaagataaccagctggctctagtggaggagaaagatgagagctgcaacaatattatgtttaagctctcgaaaatctaatgcagtaaaacgcctgtgcgttctgggttgaatgacttaatgcttccaactgaagaaagggtaacagagagaaagaaagccattcttggcttacgatgttgtggaatgttattatgtagaaaacttcttttatttccttttcttcagctacatgttcaatagactcacagatatatgacttacggcgttggtaaagaaactgaaggagattcagccttgctctttccttttctctgccttgagtcctgtatgaaatcacactcacggacttcagaagagcaggcaccacagtgcatggtttgctttagagagggtccatttcaaaaaccttcataaaaacaatgcaaaacaagaaaacaaccgaacaaaaaaaccacctatttcctggttctaaacaaatgattgtaatactagagcagttagtgggaggaccagctagggggaggatcacctaggggaggaccagctagggggaggaccagctgctgcaaagactgactgtttctcacttataataaaatgccaaatgcctccgcagatgccccaggcaaccctcagatcagccctttctgtgaagagtggcgttacctgtgcttgtttccttcttaaacttccaatttttctcttttaacacatttaacatttaactttaagcaagccagcttacattaggaagtgaaagacattttagttcccacccgtgattgaaatcattgactatatctaacaagcttaaagtctcctgtaagaactgatcaggatatacactaggacatgccaatagaatgggatctcatggtgcagtctgaagccctccaactggagagacgctaacatcatccttctcgctgatttccaaggagctatgactttggatgcatgcatctgcttggatgagatgtctcggctgcttgctttccttatgcacacgttctgttcagcttcacagcagcaatgctcacggtggaatagcttagcttagcttctgccccttctttggttcttttgaccaccatatccgtaacggctctcctactccctcagctttctttctctttgctctgacgtctatatgccaacacacttattccactgtctttaccctgcactgcagaattttacatctacctactggttaccaggttgtcccctgaacaaccttcctttgtgttttactgttattaaagtagtaatatttgtattcaaccatgtagtaatattttaagccactaaaggaatagttttacttatttagacaacagcaatttctactacatttttataagcttaaaacttacatgttttaaaacttaaaacgataaagacaataacaacattgatggagtatgatatgacagttcagaaagggttagctcttatcttccagtcgaggaaacctattgtatacaatagctggaggaatgtatgatcaaagaggccgggaaccgccgtgtaggatcgtacggctgtaacaggtataattgtttcattaatttgtcacagtcttactgtagaggaatgtaaaggcggaatctgcgtcattcctctggaaaccacagtgttgactctgtgaatctgtacgatatctttaaagtagtaactacgtagtcaaatgtgttcttgacgttgttcataactttgaataaaccatttttcaaaaccacgtgtgaccac

SEQ ID NO: 3 - DNA, 2714 bp, Canis familiaris
ttcagaggagaaatcaaaacaagattacacgaagcttcaacaacaaaggctaaaatgaagtattcaaccacgaaaatccccccagcaaagatgaacagttcagcagacaaggctttggtaaaatctcctaagctgagaaaatcccaacagaagcctgaaggagtttgccagatgtactttatgcaactgcgttctggccttatcatagaaaagacatcctgttactttaggaaagaaatcaccaaaaggtattcaccaagaacagctgaaaagtgcagaaagcaatgtctggtattcactgcctgtcatcagcagctgaacaaagatttcacctctgatgtccctatgttacagaaatgttttggaagagctaatgttccaagtatccaagaatattctgcttctctgagcacatacaatgatcaatctattactttcgtttttgaggatggaagttatgagatctatgtagaagacttgagaaaaggccaagagaaagataaggtgttattccgttattatgattcccaatccccctcacatgaaacaggtgatgatgttgatggccagacgttattggtaaacctgagtcctacaaaagataaagattttttgctgcatgccaacaacgaggaacattctgtggagctacaaaaatgtgaaaaccaattgccagaccaggccttcttcctccttcataggaagtcttctgaatgtgtttcattcgaatgtaagaacaatcctggagtgtttataggagtaaaggataaccaccttgctttaattaaagtaggagaccaaactaaggattcatatatagagaaaaccatatttaagctctcttaaatttaatgggatgaaaaaagttgtcaatcctgggttgggtagcccagatagctactgctgaagaaagaataagagataaagagatagacaacatttaagggaaataaagagtacttagtatgctatggaatgtttttcttattatgtgtaaaatatatttttataatccttcagttctgttttttattacccttgtctcactacatattcaatagtgtattattaaggagacctcagaaaatatacaacctgacttttactttttctacttgctgtcaagaaagagcttaatatctaattaagctctgctgagacctctggggacaaggaagggccttaatccaagtttcattttagacaaggatttcaaaaagccacatagagacataattttccctggttccaaacaagttcaaacagtagagctgttggtgaaaagacaatcagctctacttagactgaacatttcacaaatggaataaaacaccaaatttgtttgaagattcccaaaatttcagatacttacacatgtaggcagataatataaatatgtgaaatgacagagacaattgaaaaaattaagaaaaataaatcctgtatttgtaaagtaaataattttacctctaattgtttcatttttaaaatgctgatttttatagatggagttaaatttagcaagacattcttacattaggaagtaaaataaattttatctcagacatataattgaaatcattgggaatatctcacaaactaaaacattattttaaagcactgagacacaacataccttaagacatcaaagtaccatgggatccaagtaccatggtgaggccacaagttcccttatctactagaacctagattggacacagcattaacattacaattttaataactggctatcccttatgttcattgtatactgtctttctgactctatgctccagggatctgtaacttgatatacatgtcaccctgcacaaaatttcttataatcctttcttaccatgtgtacaagtgtttttcttgtccaccttcctgattagtctgtttactatggaatatttattgacttttcttccttcatttcttttgaccaactctgttcttgatatacattttgtgccccgtgagcttcattccatatcacctgacctctggacaccaacatatttttctattttgtctctctgatgtagaatttcacataagcctattgatgggaatattttttattaaacatctttgtattttacattactaaggtagtaaaatttattctcaacgatatatgatttttcaactactaaaggaataatttttgcttattttaagtcttaaattctactataactttttcataggcttaacattgacaataagtgataaactagtaacagaatacttatgaaatacaactgtttctgaaagtgttggctttttaattctaatttgttgagatctattggatataatgatggtggaacataagattagaaaggctgagaattactgagtgagggtaaacaattgtaaacaacatagctttcattacattgtcagttttattatgaagacaaaaataaaagcagaatatatatcatcttctctgaaaaacactaaatgttgaccatatgcatctgtatgatatactcgaaatgatttgttttaagtaactaaatatattaattacattaaattctcaagttgtattttttaaaagtatgtgaggccatgatgattttatcataaaataactattctgaaatttttaagtcaaagcaatcttaccttaatccactgatgttggtatctggggtaggtcatttgtctggtgattcaaaactaaataagatattccaaag

SEQ ID NO: 4 - protein, 270 aa, Homo sapiens
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 5 - protein, 266 aa, Mus musculus
MRPRMKYSNSKISPAKFSSTAGEALVPPCKIRRSQQKTKEFCHVYCMRLRSGLTIRKETSYFRKEPTKRYSLKSGTKHEENFSAYPRDSRKRSLLGSIQAFAASVDTLSIQGTSLLTQSPASLSTYNDQSVSFVLENGCYVINVDDSGKDQEQDQVLLRYYESPCPASQSGDGVDGKKLMVNMSPIKDTDIWLHANDKDYSVELQRGDVSPPEQAFFVLHKKSSDFVSFECKNLPGTYIGVKDNQLALVEEKDESCNNIMFKLSKI

SEQ ID NO: 6 - protein, 263 aa, Canis familiaris
MKYSTTKIPPAKMNSSADKALVKSPKLRKSQQKPEGVCQMYFMQLRSGLIIEKTSCYFRKEITKRYSPRTAEKCRKQCLVFTACHQQLNKDFTSDVPMLQKCFGRANVPSIQEYSASLSTYNDQSITFVFEDGSYEIYVEDLRKGQEKDKVLFRYYDSQSPSHETGDDVDGQTLLVNLSPTKDKDFLLHANNEEHSVELQKCENQLPDQAFFLLHRKSSECVSFECKNNPGVFIGVKDNHLALIKVGDQTKDSYIEKTIFKLS

SEQ ID NO: 7 - DNA, 16301 bp, Homo sapiens
agaatactgaaaaatgaagcctaaaatgaagtattcaaccaacaaaatttccacagcaaagtggaagaacacagcaagcaaagccttgtgtttcaagctgggaagtaaggacttaagttatctctgaatgttttacgcactatttttatctgttatcaaatatttatactccaatgtttatacatgctatcttatcaaaatctactaagaatacaagaactaaaataaaaagagatggcacataagggtataatacagcagactggttttgaaccaaccatatatagccaactttggcatatttgatacatcaccaggcaggaatagctgagataatctgaacctgtgaatagtgataaacttaattatagccttttatatcaaccactttttgctccacacacctcctgctgccaaacccccaaaaccttttccctgataaactagcaaacttttaagcaggtccacttttattcaacaaatattcattaagacctttggtggtatcaggcatgtggtcttaacttgcgactactgatgaagaagagaataacagatgaagatatttgaataatcatcttagtcaatttgtgttaccatagaaatatacctgaagctgggtaatttaaccctttttccatttagaaaaaaaagtgcagcttgctgccagcgcttatttaattttacataaacatctctttgaggctgaagcaaatctgattttcaatgcaaaaatagaatacaaaaactgttcttggagttatttctaaacagaactaccatcagcattgtctatttcagaaaaaatgagattcaccaaatgaaacttcagcaagcaactaactgttcaagaagtgttcaagaatgttaacatcacatgtaggaatgctacattttccaggatttgacattttcagcgatcaagaattactatattttatcaatgaaaataccactactaaaaacataaatagaatgatgtcttttgtttccaaagttgatatactagagagatgtaaaaataataataaaagcaagatctttcgtggcaaagttatcttaaacactgcagaagcactgccagtaattattctctgggcaaatgggaaagggttaaaaacaaaagaggtttatttggtttacagttctgcaggctgtacaagaagcatggtgctagcatctgcttctggtgagggcttcaggctatttccactcatgatggaaagggaagggaaaccagtatgtgcagaggtcacatagggggagaacagagggagaacagaagcaggagaaagaggagaggagggaccaaccttttttcaacaaccatcttgcaaactaagagtgagaattcgctcttgtgagaatggcaccaagctgttcatgagtggtctgccccatgatccaaatacctcccacaaggccccacctccaacaatggggatcaaatttcaacaggagacttggtgggaccaaaccaaccaaagccaaaccatagaaataaagcagaaccaacaaatgatgctcaagtctatctagctgcaaactcagagtttgtagtaagacagaaggtaagtaaagtcaggtggcagctatagctctgagattaaagggctgtataactctttgatccttaaaattatctgtcctaggaaagaagcatgcaaatttatttatttatttattttctgttgagacagtctcactttgtctcccaggctgaagtgcagtggcaccatctcatctcactaaaacctctgcctcctggttcaagcgattctcctgtctcagcctcccaagtagctggaattacaggcacacaccaccatacctggctaattttttgtatttttagtagagatggggtttcaccaggttggccaggctggtcttgaactcctggcctcaagtgatccacccgcctcagcctcccaaagtgctgggatcacaggcatgaaccaccgcacttggccagaaagcatgcaattttaggatctgtttttcatatgggagtagtaacacaaaagtaagttaatggagccttggtgaacaggaaagaaaatggaggataaggaatgatggtggggactggcataaccttgtccttcatgctcctttccatttctctgtacatctttaaggctagaaaagaaagcagagttaatatcatttctgtctcattctgtcttccctgaggctgtggcccacccacactgttctttggtgaccagggaaagcactaatttgactgtggtttctgaacaaccaggaaatcacagcagaaccaatccttttacttgtagctgagtcaaaatgctctctgtagaaacagtgactcagtgactttgcatccctaaattgcctctcataaaaagtacattttctttgtacctcaggaagtatgggctctttatagaaactggaatgagggagtgggctagagtttatgccagagttggtggtattgatacaaaagttaaaatgtaacccctcctttaagggaacagtggccctaactacttacctataccatttagaggacagtgataaaaataatctcaaactcatttgtatggctttacaaatacatcacattattttaattggcccttaacagatctttctttccaataatattggctcagttaaatattgtcctcccctcttatttgcaggaaatatgtaccaagacacccaacagatgcttaaaacaatggatagtaatgaaccctatatatactatgttttttcctacagatacatacccatgataatgtttaatttataagttaggcacaatgagagattaacaataaccaacaaaaaagtggagcaattagaacaacatactgtaatgaaaataatgtgaatgtctgctctctctctcaaaatatcttattgtatttcactgtgggtaactgaaatcatggaaagtaaacccacagatgggggcaagcaggatagagttactgcactaaaactgtgattatttaaacaatcctggtgaaaaatttgacagatatctctggggtatgtgacaggtgtaaactgcttctcaactcaaaatgcaagatatgtcatatcttgtttttctaaataagctgctgctctaagatgccaagacacaaggcaagaaagcacatttaattccttctcttctcttgagctaccaggatccaacacaagccacagttcacaatgctacaaaacttagtagccaaccactgcaggtgtccccacagtcagtcccacattgtggtcacccacagggcattttgcctctaatgccagcagggtacaggacttcatgacctggaggaaagcctctcacaacccttcccttccccgtttccaggaagctacttccctacttcccagggtcaggctttcctcttctcctactttctttcccccacatccatcaacactttctcttccctaaaaatccagccgtcttcattcatggatctagccagaggtcacaatatgggagccttgggatagatccatccagcaggagtattttattaggattgaacggtgttattgatattttctaattgagtcaatttttaaaaaataggatatttttaatgaaaatctaaatgtgtatagtttctctaaaaaaatccaaagatctagaaacactgggtccatatcttgcatggcaatagttggctagaactgagtagagatcaacccctttagtttgagtctatccatagaagatctgttacagtaaaggagatttgactttatcctgtagacagtagggaatcattgaaaatgttgaactgaggaatgataaaatatttttactttacagagatcacattggtgaagtgtgatgatagtagtgtgatgataaagtgaggaggcaagagtagaaacagaaactggttagaggtctactgcattaatctaagggactaatgatgaaggtgtgacctaagacaacagaatagagaggacggactagatttcagatatattcagaaggtgaaatcaaaaagacttaatgacagattgaatgaattgtaagaatggaaagaaggaaaaataaatgattctcagctttttgatttaagaattgggtagacgccaggtgttgtggctcaagcctgtaatcccagaactttgggaggctgaggcgggcggatcatgaggtcaagagattgagatcatcctggttaacacggtgaaaccccgtctctactaaaaaatacaaaaaattgcctgggcatggtggcgggcgcctgtagtcccagccacttgggaggctgaggcaggagaatggcatgaactcgggaggcagagcttgcagtgagccgagatcacgccactgcactccagcctgggtgacagagcaagactctgtctccaaaaaaaaaaaaaaaaaaaaaaaaaaaaaaaaaagaattgggtggataatgatagatagttgtactatttctcaagataggcaattcaggagaaatatattgggggcagaaaaagataaagacattttttattataaggactgccatggtttgaatgtcccctccaaaactcatgttgaaatgtaattaccattgcgatagtattaagaggtggaaccggctgggtgcggtggcttatgcctgtaatcccagcactttgggaggccaaggtgggtggatcacgaggtcaagagattaagaccatctttgccaacatggtgaaaccccgtctctactaaaaatacaaaaattagctgggcaccgtggcgcacgcctgtagtcccagctactcaggaagctgaggcaggagaattgctggaacccaggaggcagaggttgcagtgagccaagattgcaccactgcactccagcctgacaacagagcgagtctccatctcaaaaaaataaaaataaaaaaaaagagaggtgggacctttaaggggtgattaggtaataaaggctctgccctcattaatggattaatgccattgtcatgggagtgggttagttattacaggattgggtttctgataaaaatgatgaagttcagtccccatcctttctttgcctcatgtgcactctcaccatgtgtgatgtcttccaccatgtgatgacacagcaagaaggccctcatcagctacagcccctcaatcttggacttctcaacctccagaaccagacaccaaataaaactcttttctttataaatcacccagtccatggtattttgttatggcagcagaaaagagactaagaccagtgtactttagtttaaggtagctgcaggtcacccaggtagagatatccagaattcagaagatatacacagaaagagacagctatgcttcaaagtagctgggtcaaaatgacttggggagaaagaaaaacagatagaaagctcaggaaaaagaccaaagatagagttacagtttggggagtcatcagcctctaaaaaggtgattgaagtcattggagcaagcatgattgcccaggaagaaagtttaagagtgagagaagaaaaaaagtcaaaaccctgaggaaaaccaacatttatgagcagacagtagagggataactaacaaagagacaaggtgcagtagtcagagagaaacaagaagaacctagggaaactggtgacatggcataaggaaagaggggttcaaagaaagaagtggtcctgagagtccaatgccacagacaggtcaagggtagtaagcagttaagaagacaccttggtaaaatccgtgcttggggaagaaacgaaagacggagtgattcacctctgtggtttcctatcagccatagtctagttctctgagtggcagaattatagaaagatttccagggcccttcctatttactcacaagaaatccctgagggattcatgctgctgttatgattaaattagctatgggaattttcttaaaattcatactaagccattgccctcagcgagcaggcaggatagagacatggaaagtggtaacagcccttatatacatttgagctggctctttgaccccagtggagtgctctctcctgcaaagtccacatgtccaggccagctttgcactttcctctccagaacacaaccctcacactttcagcttcccaaaacaccaaagcatatgttcttcaaaattctcctgatgcaaagtgttttcaatacttcaaatgtagtaacccccatccttgaactataaccagtgcttgtcctactagatgctagcccccacatgtacatctttaactttcttatctcctaaagacttccacatcttcagtacttcctcagtattgtgaccctcacttttaactacccaagactgatatcctgttccccccagagtccacactcagtattaggcatgcctatcacgttcccatgtgatgctgactaaaatataaagaatataaggagtgagtgttctttcagttatcatattatgccttttagctctgaaaagacaatgtgatatagttgctatggtttgaatgtttccaccaaaactcatgctaaaatttaatcaccattgtaacagtgttgagaggtcagatctttttttcttttcttttctttttttttttttttttttttttgagacagagtctcgctctgtcgccagactggagtgcagtggcactatctcagctcactgcaacctccggagaggtcggatctttaagatgtaattaggtcatgagaactccaccatcataaacagattactgctgttatcatgggagtggtttggttatcaagagactgggtttgttataaaagtgagttctccggatttcctctctgacttactcactgctgccttccaccatcagatgatcctcaccagatgccagcataatattcttggatttgcctgcctctagaagtgtaagaaataaatttctatcttttttttctttttttagagacagggtcgcactctgtcacccaggctggagtgcagtggcatgattatagctcactacagcctcaaactcccaggctcaagcaatcctttggtctcagcctcccaagttgctgagactacaggagcaggccaccatgcccagctactttttttttttaaattttttgtagagacggtcttactgtgttgcccaggctggtcttgaatgtctagcctcaagcaattttcttgcctcagcctcccaaagtgctgggattacaggtgtgagccacagcacctggccaacaaatttctttgctttataaattaccaagtctatggtattctgtgatagtagcagaaaatgaattaatacaacagtcagtaaagtattgtattaaattacttgtcaggagaataagtacaatgctggctctggcactagttttctttatgaattatctttgtaagtgtaaaagcactaaaatataaatacaagataccaccgttgatatttctgcattaagaatcaatggtagagagttctagagtgggtgtgaatataattaagtggtctccccttattaaatcagttgaagatagttaaaggtacctactcatctgaattggaagcaagatgtggattaattcaaaataatagctaataaaagcttttccacttatctttaaaaagcattattttaggaaggcaaatccactggagaatttttttaactcataaaatgaatcccagagtcacaaataatattttattttctgaattactgtgactaattgaaatttgacttcattgtataaattgcttgataattcatttactacataatgttaaaaattgagatcctatgactcatgtgttacaattttcttttctacctgattctgaaagactttttatgattttccaagtagcaaatttaattttctttttgagacttagatttttctttagcaaaatgaaataattactgaataattaacttttttactttgactgatttaatttgaatttgcattttgttagtccctcaaattgtagtgtagcattttcttagatggcggtaagtggatggtggtggaaagaataggtttgattttaggcacacttcagtcacaacttctcgccagctctgtaatcttgggcaagtgtcatgactcctgagagcctcagattatcatctgtaaaatggggacaataatgcccatcttgcaagattattatgaagaattaaatggagacatatgtaaaatcattagcacagcgtttggcccactgtagacacttagcaaatggtagttattttaatatcagtaacctttgtagacttcagcaatccctccaaaattattaaaagtgttctagacaaggagaaaagattgcacatttcatatgagtaaaagatacccctcaattaggcaaaggttgatatatgttggatacaggttcacactccctcatatggaattctgaaaaccaaaatttttttctaaagacctaaagtttttcagaacttatttgacagcaaaatatgacctaaactcatttcttgttcacatcaactaactcctgatgtgaactaacataaggtctttatttcactttgtgtaaattttcctatgtttcaatataaaattaactaattttgattatggggtgctgcccccatctcttctggggtgctacatatgtgcactatatgctctatattacaatcctaaaatctaaaaaactccgaattttgaaacacagctgagttaaggatttctgataagatactgtgaacctgtacttgctttcttggtctgctacactcaggatttgtagaaggataaagatgaatggaatgaaacagtctcacaagtttttcaattgtttagaatcccaacagaaggccaaagaagtttgccccatgtactttatgaagctccgctctggccttatgataaaaaaggaggcctgttactttaggagagaaaccaccaaaaggccttcactgaaaacaggtaaggggaaccgtacattctctggcaatagtgataagtatctgtttgccttcacttaccacatctaaatatgcaattatttttcctcactccaaggttccttttggaaaatattaagaatgatgaactggtttttagctatttttaaaagggcatataaagaattttcaatagcatgagagaaatgattatgttataatgttaagtgaaaacaaaagatacataactatgtgtaaatatgtttacaatatttttaagtgtttttttaatttaaaggaaataggtttaagcattaatagtgcttgccttttgggtgttgagactgtgggtgactagttttcttcctcctgatctctaatgagtgtgcttctactggggaataagtgtgccaagaggtatcaatccctttggaccatgaagtggccaaagccctggagtcaatcactactctcagcaagcagccttaactgggaatcctcaaaggggcatttgtgcagagtggggattgatggtctatccctaatcccatccggtctgcaggtagaaagcacaaaagacatctggtactcgctgcctgtcaacagcagtctactgtggagtgctttgcctttggtatatcaggggtccagaaatatactagagcacttcatgattcaagtatcacaggtatgactggttacaggggtgatgtgggagtgaggagggaggtatgacacaggaccccggaaagtgctactccaggtagcagtcccaagtctgtgtctttaatgtgtaccagaaacttctgcccatttgcagctgatgtacccatctaatagtatccaaagtaggtgcaaggtagctgttctaataatgtacatgcattctctagttagcactccatcattattttgaaatacttgcaatggattggaatcccatggtcattagttttggtgaaaatatgttatcttttgggaaaaaaacacattttccccccaaatttcaatattttgaccatgaaagaaaagagctattatatttttaaatataattctaacactttatacattttaagtagtagttaagatggttttaaattttatcacttccttgtataaggttccaagttgatgatgacttcaagaagtgaagagatcatcattaaaaaaaaaacttggccaggtgtggtggctcacacctgtaatcccagcactttgggaggccgaggttgatggatcacctgaggtcaggagttcaacaccagcctggccaacatggcgaaaccctgtctctactaaaaatacaaaaatctgttggttgtggtgatgtgcacccgtaattccagctacttgtgaggctgaggcacaagaattgcttgaaccttgacagaggttgcagtgagccgagatcatgtcagggcactctcgcctgggcaacaagagcggctgtctcagaaaaaaaacaaacaaacaaacaaaaaaaaacccaacaaaaaacaaaacaaaacaaaaaaaccaactttacctggaaattgcatttgccatttcaagcattattcctgctataaatgtcctaaattcccagaagcaacactgaagttttctcattaagtatttctaaatcatttactttagaagttcatgtacaaattcagtatgtaacattctccttaatattttgtatactatttttttccaggccccatctgagtagtccctaaatctgctccagtttacctcagcaggctataaaaatattatcattttctatgggtattttgacactttttaaaagttgggagcactgcactaagtgtccctgttatttagccagaggagagaacagggcttatatccgcattctccaggactgtcagaaacttctgggtaactccatactttatagatgaacatttatttatccatgtctatcaaaatacagtcccttctttaccacagtccccagaccaagtggtgtcatgaagtgaaggtttcctcaagcaatgtctgttgctttccattttctttttccccaccaaaccctcccataagcacactcacatggcctcccagggccttcatgcaggacactagcacctttaatcccattcaagggtcacatctcagccttttagctatgtaaaacttgtatcaaaggctttaatctagccaacagtgacatacaaaatgcaactcaaattgcaaaaatgttttttgagggagcattttttaaaaaggttgccaaaagaattgtgcctgacaaatttttgaattcttaacaacaggaatttcacctattacagagtatcttgcttctctaagcacatacaatgatcaatccattacttttgctttggaggatgaaagttatgagatatatgttgaagacttgaaaaaagatgaaaagaaaggtagattattttctttttctataataatgtaataatgactaataaaagtaaaacattttaataaatctaccaaactttaaacaatggattaacttagacatggcaaacaggtcatgctgtgtgctaactctaatgcattggcagtgggtgcctgaatccacttctatgtcaagatggattcagaagctttgtcacgttcatcagagaagaatgctgacattgaaatgcagtatctgttgtgaaagatggaaagaggggccacatgtatttgctatctctaaattatccaccagatctttctagccctattagtatcaccaaagtacaacctttatgccactgctatgaaaaatattttaaatattttaataacaactatattggagaattcatcagagcatattcgtgcatttttgagacctttctcaaaaccacaaaagatttgtttttattgatatataataaaaggggaggatattttctgatgttatgtgtgtgttggaactgaaaacttaacaaaattgtgtctcaccagagggattttatgcattctctttcagataaggtgttactgagttactatgagtctcaacacccctcaaatgaatcaggtaatttggagggctgggtagctgtagtgcttgaattcttaagtatggggtaaagataaatccaaaaaaatcctttttgccccataggaaaaaagatagtctcagaaggttatctccaacccaagctcatggtaaaaccttagattctcagaggatgcattctcatccattttacagcttaatcgttaggataaagttttggctacacttaatacagatccaaaacaagagtggtttatgctagataaaactttctgtcttagccagggctgcaatggctcaacaagttgtccagggaacccatctccttctatcttgtggttctgccaacatctacatggttcaagagtacttgggagcatgacaatgcctggtcagcaggacacagaaaaacagatgtatgcattctatttccctttaagggtacaacctagaagttgcacctaacacttcctcttatctcacattggtcagaactcagttgtattactacacttagccataagaaggcaagctggggaatgttctattactatcccaaaagaggaatgaatattgggtgacactatgagtggctttacatgcagacaggaaagctgatgccccgagaagtaaccattagggtcacaactctccaattaaaaagcatataagaaaactaatttctctccggtttataagtaccctctacttatcagtgcctcttgaacttgggtacaaatacttctaagatacctgttagtgaatgaagtaattattttatgtaacatcttagacttttttcctgggtgttgaattcatttaaataaagatgagttacagttcttaactttatcatttatactttcttaattgtaaggtgacggtgttgatggtaagatgttaatggtaaccctgagtcctacaaaagacttctggttgcatgccaacaacaaggaacactctgtggaggtaaaaaaaaaaaatttatctatatctatatatatgattacagaactgtcatgaatgactccaccaacttgtgtagatttgggtgttggggtattttgcagaaggatttttttgtgtgctattgtatataaagcacaatcacttactacaaatgacttgcttctagagtaaggaagggaggaaagtggcatgtatggctttgctattgaaagtctgctctctgccagcagcagagcatcacctgggagcctgttagaaatgcaggattctaagccccccagaaataaatcagaatcagcatattttaataaatctccaggaaattcatatgtgccttaaagtttgagaagcactgttacatagaatgatctcaaatcctttctgtttggctttaggaactactatgttcctctaggaaacgtttctttgtataatatcattttcttaaacaaatacaaaaaaaatttccattctgagcctgcttaagggagagtcataagcgtttccatgtgccaagatttataaataacctaacatctacattctgaacatagaattaattgcatattcttctttagttagcaaatggcattattacattggtcatgatgtgtaattataactattttgacaaacaagccaaaggtagaggcatggaagaaaacagaagagagcataatatagaagataaattatacaaacacagaaaaatataaagctaactaacacagccatgtaaggccttattaacataaatagtggatagtaaccagcatcttcctaagcaaaacattcctaaacatatataatctagtgtttggcatgtataactactcaataatagagctacttagactgagctctattatatagcaagcactgggctaaatgttttacaaatattattttatttaatactcacagtaactctacaatggagttgttgtattggtcaactgttgctatgtaacaaaccatcccaaaacttagtgcctcaaaataataatcatttatatttactcatgtctgtagatattttgaggattgactgatctagaaaactggttagactaaaaaaaatgaggcttgagagataaagcaacatccccaagatctcagagatagtacatgtcaaaagaagggtacaaatccagaactgttgcatccagatccaatcaccctaatccatactgcctctctatgttgactatcagtgaatcaaatcaaatccacccactacccctctggccctctactcacttctccccctcttccctccacaactcactaagcaagcttgctagaaatatcaagtcataaataagtattcccctttagtttccaatacaggcaggtaaagttgactctgaacttcccattcacatatggattgctttctctcttgtttcctcagctccataagtgtgaaaaaccactgccagaccaggccttctttgtccttcataatatgcactccaactgtgtttcatttgaatgcaagactgatcctggagtgtttataggtgtaaaggataatcatcttgctctgattaaagtagactcttctgagaatttgtgtactgaaaatatcttgtttaagctctctgaaacttagttgatggaaacctgtgagtcttgggttgagtacccaaatgctaccactggagaaggaatgagagataaagaaagagacaggtgacatctaagggaaatgaagagtgcttagcatgtgtggaatgttttccatattatgtataaaaatattttttctaatcctccagttattcttttatttccctctgtataactgcatcttcaatacaagtatcagtatattaaatagggtattggtaaagaaacggtcaacattctaaagagatacagtctgacctttacttttctctagtttcagtccagaaagaacttcatatttagagctaaggccactgaggaaagagccatagcttaagtctctatgtagacagggatccattttaaagagctacttagagaaataattttccacagttccaaacgataggctcaaacactagagctgctagtaaaaagaagaccagatgcttcacagaattatcattttttcaactggaataaaacaccaggtttgtttgtagatgtcttaggcaacactcagagcagatctcccttactgtcaggggatatggaacttcaaaggcccacatggcaagccaggtaacataaatgtgtgaaaaagtaaagataactaaaaaatttagaaaaataaatccagtatttgtaaagtgaataacttcatttctaattgtttaatttttaaaattctgatttttatatattgagtttaagcaaggcattcttacacgaggaagtgaagtaaattttagttcagacataaaatttcacttattaggaatatgtaacatgctaaaacttttttttttttaaagagtactgagtcacaacatgttttagagcatccaagtaccatataatccaactatcatggtaaggccagaaatcttctaacctaccagagcctagatgagacaccgaattaacattaaaatttcagtaactgactgtccctcatgtccatggcctaccatcccttctgaccctggcttccagggacctatgtcttttaatactcactgtcacattgggcaaagttgcttctaatccttatttcccatgtgcacaagtctttttgtattccagcttcctgataacactgcttactgtggaatattcatttgacatctgtctcttttcatttcttttaactaccatgcccttgatatatcttttgcacctgctgaacttcatttctgtatcacctgacctctggatgccaaaacgtttattctgctttgtctgttgtagaattttagataaagctattaatggcaatatttttttgctaaacgtttttgttttttactgtcactagggcaataaaatttatactcaaccatataataacattttttaactactaaaggagtagtttttattttaaagtcttagcaatttctattacaacttttcttagacttaacacttatgataaatgactaacatagtaacagaatctttatgaaatatgaccttttctgaaaatacatacttttacatttctactttattgagacctattagatgtaagtgctagtagaatataagataaaagaggctgagaattaccatacaagggtattacaactgtaaaacaatttatctttgtttcattgttctgtcaataattgttaccaaagagataaaaataaaagcagaatgtatatcatcccatctgaaaaacactaattattgacatgtgcatctgtacaataaacttaaaatgattattaaataatcaaatatatctactacattgtttatattattgaataaagtatattttccaaatgt

SEQ ID NO: 8 - DNA, 11104 bp, Mus musculus
  misc_feature 6352, 6353, 6354, 6355, 6356, 6357, 6358, 6359, 6360,6361, 6362, 6363, 6364, 6365, 6366, 6367, 6368, 6369, 6370, 6371,6372, 6373, 6374, 6375, 6376, 6377, 6378, 6379, 6380, 6381,6382, 6383, 6384, 6385, 6386, 6387, 6388, 6389, 6390: n = A,T,C or G
  misc_feature 6391, 6392, 6393, 6394, 6395, 6396, 6397, 6398, 6399,6400, 6401, 6402, 6403, 6404, 6405, 6406, 6407, 6408, 6409, 6410,6411, 6412, 6413, 6414, 6415, 6416, 6417, 6418, 6419, 6420,6421, 6422, 6423, 6424, 6425, 6426, 6427, 6428, 6429: n = A,T,C or G
  misc_feature 6430, 6431, 6432, 6433, 6434, 6435, 6436, 6437, 6438,6439, 6440, 6441, 6442, 6443, 6444, 6445, 6446, 6447, 6448, 6449,6450, 6451: n = A,T,C or G
aaacctgaaaaatgagacctagaatgaagtattccaactccaagatttccccggcaaagttcagcagcaccgcaggcgaagccctggtcccgccttgcaaaataagaagtaagagcgctctctttaaccactattaaatctattatcaaatatttgttatttcaacatgaagacgggcgaccttaccaaaaactctcaagtatacaagagctgaaaagaaagagagacagcttgttgttttcttaaacagacttcacagcatcaccgttgttatttggtacaacataggacagttgaccacttattcttggactagtaacaagaagggtagcagagctctggtggacagaacggaagttagagggtgatgggaaaggaaagcgatgtccccttctgcccttcccgaggtgctggactgctcacacacttctttcaaagccaggatttaaaagaaaaaaaattgacagtggtttctgaatggccagaaaaaaatcacagttaaattaatcctttcatttgcagttatgtcataatgctctgtgcagatataatgactcagtgcaagtgactcggtgttcccaagtttcctcctattaaagaatgcactgtcttcagatccaagaaaacgaggccctttatcggcactgccttgtggagtagacatgccagaatgcctgacattggtgcccgcgcttgcttcgaatactgagatcttacaattcacatgttctagttttcatctccatctcaccctggaatactatttacgatgtccagaacacaatggacttttctgcatgggacttaaatttttcttgggctaagtaaaatgagaattaacaaataactcgttaatctttttagttccgttgcctgaatttgcatttgactgctccctcagatggcagtgctcacttacttcttcccctaccaaataagactctgtgtagctcacactgatctggaaactcgcaaccctcctgctttagcctcttgggtactgggactagaggcacgaccacatctggccagtggtccttttctttgatggaggtagagtctaatgagtagctttgatattaaagatattgcattaattaattttacaccgctatggccaaatacccagcagaagcaacataaaggcttcatagcttagaagatacaggccattaggatacagcagggatggtggctgaaccagtttgatctctggtagccttagcttccagggacttctcatatggcaccgcccaagagtgggagatgaaggaccaggctataatctgagactgtcccccacggtctaggtatatcagcaaggccccaaaagtttcacaatctcccaaagagtttcactagtttgggaccaagtgctcaaggacaagattctgtgggaacatttaactgtaacacacttcttagcacaactctgagctatgtaattttgggcagtgccatgaaccctaaaagcctcgaattatcattgtaaaactaagataaaactttgcaagataatcatgaatggctaagttaaaagacacataaaagtaattagcagagtgtttggcctcgtttttatatgcagtcaatggcagttcttttagtatcaataacctttatggcttacagccatcctccaaaaattatgacgagcagaaatctcctatggatgttaacgtgcccctcagttaggcaaagattggtccatactgaatgtagacttggtccttcaaatggaattatgaaaacaaaacaccaaaaaaaaaaaaacccaaaaaaaaacaaaaaaccccaaatttctaaagacctaatgtctttagaccatgtggcagcaaaatgcaactccagcctcttgtctatcaaatcccaaggtctggaacggctcattttaaaaggcaaattgattgaattcagaaccactcaaggcattcgtgaggcatgtctttaggtatgtgtctatgacagcattctctggaaaggattggcttagacccaccctgaaaatgagtagctgcgacatcccctgggctggatccagaactaattaaggagaatacggatatagacacaccttccctccataaagaacctgagacacaagcaaaactaatccctttctcctttaagttgctgcttgtcaaagacttggtcacagtaaggagaaaagactatttcattttgcatatcctatctgtggcttgctgtaaaattaatgaagttaactaagtagtgctgttccagcctctgttggaggcacgtgtatattataggatatgacctacataaatctacctctttttttcctattgttaggatcccaacagaagaccaaagaattctgccatgtctactgcatgagactccgttctggcctcaccataagaaaggagactagttattttaggaaagaacccacgaaaagatattcactaaaatcgggtaagtacatttctggaaagactgatgggcatctatttgcttttactttccatacacacacacacaacacacacacacacacacacacacacacacacacacacacacgcacacacacacacaattgtttttcttacctaaattctccttgaggaaaagaatacagatgataatgtgttggccattttagaaagggtttttctattttagaatttctaaataaatattcaaaaagtctattctagaatccactttagaaatggatacactggtgagtaaaaacagaagacacaagattatgtgtaaatatgtaatcatgtggtgtgatattttgtaatatagaggaaacaaactaaatggttcatagtggctgctttggaaggcagtaactgtggtgagtggctttctgtttatttgtttgtttgtttcttcctttggtgtgctctactggagtgcaaatggtcttcactttacgtcattattaaggaggaattgatcccattagaccattgggtggccacagccctaggatcacttaccaccgtcgccatattggccatcaaaaaagatagcatattggcacaatgaagattgctgactcagcccaagccccactactgtcttgcaggtaccaagcatgaagagaacttctctgcctatccacgggattctaggaagagatccttgcttggcagtatccaagcatttgctgcgtctgttgacacattgagcatccaaggtatgactggtcataggggatgtgtggggtgaggtgggagatagcacatggggccttggcaagggctattgcatagagcaaactcaggagtatgtcttcatatgtattagaaacgtttgtttcctgcttgggtaccagttcagtggttatccaaattgggtgtggtggagctgtgcctatttaatctatggttagcattccacttgtattttgaaatacttgctataggttgggagcatgtcttcatcagtcctgataaatgtaatatccttggagagaggaaaaaaacaaagcaccagtccccacatttcaaaatgttcaccatgaaagaaaataaaggaactatcttttaaaataacatgatgtggcttgaagagtagtttaaagtttttgaccatttttgctgcgttctaagtaaaatggtaattatcaagaagtgaagagaacccagggcgtgtagttcagtggtagaacatttgcctcatgtgccaggctctgcatgtagtctctatcaatgtgggaaaaattcaaagtgagggttgggcataatggcatataatctccatcccagaatgggggaggcaggtgaattccttgggttcaggtctgcctggtctacacagtgagtcccagaacagttagagttatatagtgagaaactgtctcaaagacaaacaaaaggccaaattaaataattaacaaattagcagccccatcactaaggaatcgtgattaagaatcacatattgccattctaaatatatattatttttatatgtgatcttaatataaaacaagcttagaatgtatttatgtatagtgtgtatatatattatatatatatatatataatatatacttagtatatgtttaatataaatacattaacctcctggtcaatattcagtttctcacaaactggatttaaattactttagatgtttagatgtttagatgtttagatgtttagatgttcacatgcaaattctttctattttgtacagtaaatttgaatgtaggaccctcagatgggtccaagtctgcttcagtttacccatatatgcttttaaaatgttatcaagtgctctgtatgtgtttcgattgtccgggcaaaagcagaactagtttacatctgcatccccctagaccagtgactcaatcttcctaaatgctgtaaccctttaacatagttctgcacgctgtggtgaccccaaccataaccttactcttgttgctacatcataactgcaactttgctactgttatgaatcctaatgcaaatatctggtctgtaagatatctgatatgcaatccctgtaaggggatcatttaacccacaaaaggtctggaccaccaggttgagaaacactgccctagagtgttggaaatctctaagtgatcatgctttataaaggggaatacattcacctatgtcttccaaaacatgaccattctttgcagctgtcccctagactgtgggaaggtaagaaaaggagctaccctcctcgtcctcaccccaactgtgcaggaaggtaacacctttaatcccaccctggtatcagctatggtacttagcttttgcctatgttacaatgacaaagtagagctcacactaaagaccaaaactcttcagaggaagtaattcatataaataaccaagagaacggttcatagaatcctttgtgttttttattcttaatgacaggaacttcacttttaacacagtctcctgcctccctgagtacatacaatgaccaatctgttagttttgttttggagaatggatgttatgtgatcaatgttgacgactctggaaaagaccaagagcaaggtagagtatctcctcacgtctatacttagtaatgactggcaaggtttaaaaaaaaaaaatctaccaagttatcaacagtggattaactgagctattcaacagcagcaagtttcaccatctttggtaattttagtccttcctatgtcaagaaggtctcagtgtagtcacattcttcagggaataaatcagacattgagaaatacctattgagaaatacagaaagagggactacatgtatctacaaatgctgaataataagatcttgttcaccttttcagtgtccccaaagcatagtcttttgtgaaagattatccctttgtcataaacaataccacctgcatattggggaatccatcagagcagaatatattttaagatctttctaagaatcactaaagttccgcttttttggtatatgacaaaaggagagggagtttgtgttccccgcccagcccagccccaggacgggatggaaccgaaaccttttgatacgttcctctgtcacccagtgacacccaatgttgtctttcagaccaggtgctactacgctactatgagtctccctgtcctgcaagtcaatcaggtaatgtagaggcagggggagggggtggtgtttgcattctcatccagaacaaagctgaatctgaagtagcccgtggttatcatgaggttcagaaggttatcttcaaccccacactcattgtaaaaccttacctcatctacttctgaattagtaggagaaaagcctagctgcacttataatagtacagccataatagtacagcccatctcagccaagactcaaaagattctgctcaaaggttgcccagaagcagttccttctacatcatcagtccatccatgcataatggctcaccactatggccagtcagcaagagacaggaaggcaggtgaatacatatgtcttccgcttcagggcacaattgtgtaccacgtgtctgcttatcttcagttggtcagaacttagtctgagagcaacaggaggagaaaagttctattatctgttatctattagaggacagctctcagcagcttcccatgaacagagggaagcaggtgaccaaagcagtatccattggcatcagttgaattacaaaagatcagaacaacaacagaaagtaaaaactaaaacctctcctcagaaagatttagaaagactcttgggctggccttggtggtgcacatctttaatcccagcactcaggaggccaaggcaagcagatctctgagttcaaagccagcctgacctacagagtgagtttcaggacagatagggccacacaaagaaaccctatcttgggggaaggaagaaataaaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagggaagggaagggaaggaagaagggagggagggaggaagggaaggaggaaagaaggaagagagggaggaaagaaggaaggaaggaaggaaggaaggaagtaaggaaggaaggaaggaaggaaggaaggactacctggtatttgggtgcaaatacttttaaatggattagtgagctattttcatttgcaactgaaattctttgagttaagttaagatgagttacaagacttagaatttttgatttgtaatttctttgtcttacttttaattgataggtaaaattgtattaatcatgaaccacagatgatttttgctttggagcaagcgatattttttttaaaaagattttatttctatttaatgggtatgaatgttttgcttgcatgcatgtaagcgcttgagatgcttatagttgtgtagaaagctagaagacagcactggatgtcctgtaacttatgggctctgggaactgaatgcaggtcctcttgcaagggcatcaagtgctcatgacgaaggaagtatctctctagtcccatagcaggagttttttttttttttaaagatttatttattgattatatgtaagtacactgtagctgtcttcagacactccagaagagggcatcagatttcattagggatggttgtgagccaccatgtggttgctgggatttgaactctggaccttcagaagagcaatcgggtgctcttacccactgagccatctcaccagcccatagcaggagttttgaggtatctattgtctctgtctaatagagagatcaaatgaggccaagtggcagctgtatcacctcaccttataattttcattttaaggcgacggtgtggatgggaagaagctgatggtgaacatgagtcccatcaaagacacagacatctggctgcatgccaacgacaaggactactccgtggaggtaacagaaatatgaccctatgacggccacacttggttctacctgttaacgtaaaatagatgttagatgttagggcaattttgaaggcagaagtacacactgttgaaaataaactacagtcagtcacttctaatgacttgcttgggagtagggaagggagagaagtgtggtcttgttgctacaagtctggtctccagcaacaacacagaatcactggagaacttgtcagaaagcaggatttccggctccccccacatccgaatcagatctaaagtctccaggacaggaacttcatcccttcaccacagtttgagaggcacttctgcacagagtgagtcacagcctttccatttggctatgttcttccagaattgttttaggattatttattttatgcatatgaatgtactgtcactgtgttcggtcacaccagaagagggcatcagatcccattacagatggttgtgagccaccatgtggttgctgggaattgagctgaggacctctggaagagcagttggtgctcttaaccgctgagccatctctccagccctggagatatatagtttctgactccaagtctgtcaaggggaaggcagcaaatgtgtccatacatcagcagtttggggcagcattacattctacatctggtccgttcttgctcagcaaacagcaggtgtacatcagccaggacgaaaaaaaaaattctgatcatgtaaagaacaaacaggccagagtgagaaggaaggaaggaagggaggaaggacatgggagaacataataacgaagatgatttctataaatatataccatttcaaagcaatccagccacataagacatcaacacaaacagtccacaaagaccagcactcttctcaggagaatactctcaggtgtacatgatgcaaaatctagcagaaagaaagcctcaaaagtaacagtgggctgccatggcacatcaagttcttgcttaccatttcctaagccctgtctctttaatcctcacagcaaatttatatggaaaaattgccttggtcagctattgtgcaacaaactatcacaaaactcattggattaaaataataatcacatataggtcctcagcaactgagagctcattccgacgacctgagttggctccccagtattcattggcaggcgactcacaacacctgtagctctcgctccaagacagtcaggtgcctctggcctccatagacatctacacttatgtgcacacactcagacatagccacatacacataatttaaaataaattcttaaaaataacacaaattaaaaacaataatctgtatttaaatgagcttaatttaattgaacaacttagtttaacaatcaacttaattaaaatatgtctgtatttaaactgacaatgaacgaacctagagggcgtgtccacagagagttctggctaaactgaaaagtccaggtttactagatacatactagacagacatgttctcatggcaaaaacagaggggaacaaatctggatgttttgcggtagtttgttgcacagaagctgaccaaggtaattattccatataaacttgctgtgtgaattaaagagatggggtttgtgacgtatgaaggaaattgacctggtcagtctctgccaagctgcttatccactctccacaaatcgctttcacctcttccatgcagcctctctattccctccactgctcatttctccctcctcgactcagtacaaacttcctagaaagttcaagctataagtgatcagtccccccatggatgctgacccaggaaggcaatggtagctctggactccccattcacaccttggtttcttctcttcttccctcagcttcaaaggggtgacgtctcgcctccggaacaggccttcttcgtccttcacaaaaagtcctcggactttgtttcatttgaatgcaagaatcttcctggcacttacataggagtaaaagataaccagctggctctagtggaggagaaagatgagagctgcaacaatattatgtttaagctctcgaaaatctaatgcagtaaaacgcctgtgcgttctgggttgaatgacttaatgcttccaactgaagaaagggtaacagagagaaagaaagccattcttggcttacgatgttgtggaatgttattatgtagaaaacttcttttatttccttttcttcagctacatgttcaatagactcacagatatatgacttacggcgttggtaaagaaactgaaggagattcagccttgctctttccttttctctgccttgagtcctgtatgaaatcacactcacggacttcagaagagcaggcaccacagtgcatggtttgctttagagagggtccatttcaaaaaccttcataaaaacaatgcaaaacaagaaaacaaccgaacaaaaaaaccacctatttcctggttctaaacaaatgattgtaatactagagcagttagtgggaggaccagctagggggaggatcacctaggggaggaccagctagggggaggaccagctgctgcaaagactgactgtttctcacttataataaaatgccaaatgcctccgcagatgccccaggcaaccctcagatcagccctttctgtgaagagtggcgttacctgtgcttgtttccttcttaaacttccaatttttctcttttaacacatttaacatttaactttaagcaagccagcttacattaggaagtgaaagacattttagttcccacccgtgattgaaatcattgactatatctaacaagcttaaagtctcctgtaagaactgatcaggatatacactaggacatgccaatagaatgggatctcatggtgcagtctgaagccctccaactggagagacgctaacatcatccttctcgctgatttccaaggagctatgactttggatgcatgcatctgcttggatgagatgtctcggctgcttgctttccttatgcacacgttctgttcagcttcacagcagcaatgctcacggtggaatagcttagcttagcttctgccccttctttggttcttttgaccaccatatccgtaacggctctcctactccctcagctttctttctctttgctctgacgtctatatgccaacacacttattccactgtctttaccctgcactgcagaattttacatctacctactggttaccaggttgtcccctgaacaaccttcctttgtgttttactgttattaaagtagtaatatttgtattcaaccatgtagtaatattttaagccactaaaggaatagttttacttatttagacaacagcaatttctactacatttttataagcttaaaacttacatgttttaaaacttaaaacgataaagacaataacaacattgatggagtatgatatgacagttcagaaagggttagctcttatcttccagtcgaggaaacctattgtatacaatagctggaggaatgtatgatcaaagaggccgggaaccgccgtgtaggatcgtacggctgtaacaggtataattgtttcattaatttgtcacagtcttactgtagaggaatgtaaaggcggaatctgcgtcattcctctggaaaccacagtgttgactctgtgaatctgtacgatatctttaaagtagtaactacgtagtcaaatgtgttcttgacgttgttcataactttgaataaaccatttttcaaaaccacgtgtgaccac

SEQ ID NO: 9 - DNA, 38 bp, Artificial Sequence
  primer
gaattctgaaaaatgaagcctaaaatgaagtattcaac

SEQ ID NO: 10 - DNA, 38 bp, Artificial Sequence
  primer
gggcccagtttcagagagcttaaacaagatattttcag

SEQ ID NO: 11 - protein, 16 aa, Homo sapiens
MKPKMKYSTNKISTAC

SEQ ID NO: 12 - protein, 16 aa, Homo sapiens
CYFRRETTKRPSLKTG

SEQ ID NO: 13 - DNA, 20 bp, Artificial Sequence
  primer
cacccctcaaatgaatcagg

SEQ ID NO: 14 - DNA, 20 bp, Artificial Sequence
  primer
ggagctccacagagtgttcc

SEQ ID NO: 15 - DNA, 20 bp, Artificial Sequence
  primer
accacagtccatgccatcac

SEQ ID NO: 16 - DNA, 20 bp, Artificial Sequence
  primer
tccaccaccctgttgctgta

SEQ ID NO: 17 - DNA, 77 bp, Artificial Sequence
  misc_feature 26, 27, 28, 29, 30, 31, 32, 33, 34, 35, 36, 37, 38,39, 40, 41, 42, 43, 44, 45, 46, 47, 48, 49, 50, 51, 52: n = A,T,C or G
  synthetic oligonucleotide
caggtcagttcagcggatcctgtcgnnnnnnnnnnnnnnnnnnnnnnnnnnngaggcgaattcagtgcaactgcagc

SEQ ID NO: 18 - DNA, 25 bp, Artificial Sequence
  primer
gctgcagttgcactgaattcgcctg

SEQ ID NO: 19 - DNA, 25 bp, Artificial Sequence
  primer
caggtcagttcagcggatcctgtcg

SEQ ID NO: 20 - DNA, 29 bp, Artificial Sequence
  primer
ggatcctaatacgactcactatagggaga

SEQ ID NO: 21 - DNA, 18 bp, Artificial Sequence
  primer
aaatgaatcaggtgacgg

SEQ ID NO: 22 - DNA, 18 bp, Artificial Sequence
  primer
tggcagtggtttttcaca

SEQ ID NO: 23 - DNA, 18 bp, Artificial Sequence
  primer
agtgattgagagtggacc

SEQ ID NO: 24 - DNA, 18 bp, Artificial Sequence
  primer
attctcagccctcttcaa

SEQ ID NO: 25 - DNA, 20 bp, Artificial Sequence
  primer
aaatcccatcaccatcttcc

SEQ ID NO: 26 - DNA, 21 bp, Artificial Sequence
  primer
catgagtccttccacgatacc

SEQ ID NO: 27 - DNA, 16 bp, Artificial Sequence
  primer
gctctgttcccaggac

SEQ ID NO: 28 - DNA, 20 bp, Artificial Sequence
  primer
gttcatacaccttccggttg

SEQ ID NO: 29 - DNA, 18 bp, Artificial Sequence
  primer
agtgtgaaggtgaagtcc

SEQ ID NO: 30 - DNA, 16 bp, Artificial Sequence
  primer
ggtcagttggatttgt

SEQ ID NO: 31 - DNA, 16 bp, Artificial Sequence
  primer
ggtcagttggatttgc

SEQ ID NO: 32 - DNA, 16 bp, Artificial Sequence
  primer
tgctgctcctgctgac

SEQ ID NO: 33 - DNA, 16 bp, Artificial Sequence
  primer
gctaccacttccacct

SEQ ID NO: 34 - DNA, 18 bp, Artificial Sequence
  primer
ccagatgcaatcaatgcc

SEQ ID NO: 35 - DNA, 17 bp, Artificial Sequence
  primer
ctgaacccacttctgct

SEQ ID NO: 36 - DNA, 20 bp, Artificial Sequence
  primer
gaatactgaaaaatgaagcc

SEQ ID NO: 37 - DNA, 21 bp, Artificial Sequence
  primer
gactcacaggtttccatcaac

SEQ ID NO: 38 - protein, 107 aa, Homo sapiens
MARAALSAAPSNPRLLRVALLLLLLVAAGRRAAGASVATELRCQCLQTLQGIHPKNIQSVNVKSPGPHCAQTEVIATLKNGRKACLNPASPIVKKIIEKMLNSDKSN

SEQ ID NO: 39 - DNA, 1103 bp, Homo sapiens
cacagagcccgggccgcaggcacctcctcgccagctcttccgctcctctcacagccgccagacccgcctgctgagccccatggcccgcgctgctctctccgccgcccccagcaatccccggctcctgcgagtggcactgctgctcctgctcctggtagccgctggccggcgcgcagcaggagcgtccgtggccactgaactgcgctgccagtgcttgcagaccctgcagggaattcaccccaagaacatccaaagtgtgaacgtgaagtcccccggaccccactgcgcccaaaccgaagtcatagccacactcaagaatgggcggaaagcttgcctcaatcctgcatcccccatagttaagaaaatcatcgaaaagatgctgaacagtgacaaatccaactgaccagaagggaggaggaagctcactggtggctgttcctgaaggaggccctgcccttataggaacagaagaggaaagagagacacagctgcagaggccacctggattgtgcctaatgtgtttgagcatcgcttaggagaagtcttctatttatttatttattcattagttttgaagattctatgttaatattttaggtgtaaaataattaagggtatgattaactctacctgcacactgtcctattatattcattctttttgaaatgtcaaccccaagttagttcaatctggattcatatttaatttgaaggtagaatgttttcaaatgttctccagtcattatgttaatatttctgaggagcctgcaacatgccagccactgtgatagaggctggcggatccaagcaaatggccaatgagatcattgtgaaggcaggggaatgtatgtgcacatctgttttgtaactgtttagatgaatgtcagttgttatttattgaaatgatttcacagtgtgtggtcaacatttctcatgttgaaactttaagaactaaaatgttctaaatatcccttggacattttatgtctttcttgtaaggcatactgccttgtttaatggtagttttacagtgtttctggcttagaacaaaggggcttaattattgatgttttcatagagaatataaaaataaagcacttatag

SEQ ID NO: 40 - protein, 114 aa, Homo sapiens
MSLPSSRAARVPGPSGSLCALLALLLLLTPPGPLASAGPVSAVLTELRCTCLRVTLRVNPKTIGKLQVFPAGPQCSKVEVVASLKNGKQVCLDPEAPFLKKVIQKILDSGNKKN

SEQ ID NO: 41 - DNA, 1547 bp, Homo sapiens
ggcacgagccagtctccgcgcctccacccagctcaggaacccgcgaaccctctcttgaccactatgagcctcccgtccagccgcgcggcccgtgtcccgggtccttcgggctccttgtgcgcgctgctcgcgctgctgctcctgctgacgccgccggggcccctcgccagcgctggtcctgtctctgctgtgctgacagagctgcgttgcacttgtttacgcgttacgctgagagtaaaccccaaaacgattggtaaactgcaggtgttccccgcaggcccgcagtgctccaaggtggaagtggtagcctccctgaagaacgggaagcaagtttgtctggacccggaagccccttttctaaagaaagtcatccagaaaattttggacagtggaaacaagaaaaactgagtaacaaaaaagaccatgcatcataaaattgcccagtcttcagcggagcagttttctggagatccctggacccagtaagaataagaaggaagggttggtttttttccattttctacatggattccctactttgaagagtgtgggggaaagcctacgcttctccctgaagtttacagctcagctaatgaagtactaatatagtatttccactatttactgttattttacctgataagttattgaaccctttggcaattgaccatattgtgagcaaagaatcactggttattagtctttcaatgaatattgaattgaagataactattgtatttctatcatacattccttaaagtcttaccgaaaaggctgtggatttcgtatggaaataatgttttattagtgtgctgttgagggaggtatcctgttgttcttactcactcttctcataaaataggaaatattttagttctgttttcttggggaatatgttactctttaccctaggatgctatttaagttgtactgtattagaacactgggtgtgtcataccgttatctgtgcagaatatatttccttattcagaatttctaaaaatttaagttctgtaagggctaatatattctcttcctatggttttagatgtttgatgtcttcttagtatggcataatgtcatgatttactcattaaactttgattttgtatgctattttttcactataggatgactataattctggtcactaaatatacactttagatagatgaagaagcccaaaaacagataaattcctgattgctaatttacatagaaatgtattctcttggttttttaaataaaagcaaaattaacaatgatctgtgctctgcaaagttttgaaaatatatttgaacaatttgaatataaattcatcatttagtcctcaaaatatatacagcattgctaagattttcagatatctattgtggatcttttaaaggttttgaccattttgttatgaggaattatacatgtatcacattcactatattaaaattgcacttttattttttcctgtgtgtcatgttggtttttggtacttgtattgtcatttggagaaacaataaaagatttctaaaccaaaaaaaaaaaaaaaaaaa

SEQ ID NO: 42 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 43 - DNA, 1639 bp, Homo sapiens
acaaactttcagagacagcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttgaatataaggattttcctagatattgcacgggagaatatacaaatagcaaaattgggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagataaacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgtcaaagt

SEQ ID NO: 44 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 45 - DNA, 757 bp, Homo sapiens
ggaaccgagaggctgagactaacccagaaacatccaattctcaaactgaagctcgcactctcgcctccagcatgaaagtctctgccgcccttctgtgcctgctgctcatagcagccaccttcattccccaagggctcgctcagccagatgcaatcaatgccccagtcacctgctgttataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagacttgaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtacaaaaaaaaaaaaaa

SEQ ID NO: 46 - protein, 107 aa, Homo sapiens
MARATLSAAPSNPRLLRVALLLLLLVAASRRAAGAPLATELRCQCLQTLQGIHLKNIQSVKVKSPGPHCAQTEVIATLKNGQKACLNPASPMVKKIIEKMLKNGKSN

SEQ ID NO: 47 - DNA, 1110 bp, Homo sapiens
gacagagcccgggccacggagctccttgccagctctcctcctcgcacagccgctcgaaccgcctgctgagccccatggcccgcgccacgctctccgccgcccccagcaatccccggctcctgcgggtggcgctgctgctcctgctcctggtggccgccagccggcgcgcagcaggagcgcccctggccactgaactgcgctgccagtgcttgcagaccctgcagggaattcacctcaagaacatccaaagtgtgaaggtgaagtcccccggaccccactgcgcccaaaccgaagtcatagccacactcaagaatgggcagaaagcttgtctcaaccccgcatcgcccatggttaagaaaatcatcgaaaagatgctgaaaaatggcaaatccaactgaccagaaggaaggaggaagcttattggtggctgttcctgaaggaggccctgccttacaggaacagaagaggaaagagagacacagctgcagaggccacctggcttgcgcctaatgtgtttgagcatacttaggagaagtcttctatttatttatttatttatttatttgtttgttttagaagattctatgttaatattttatgtgtaaaataaggttatgattgaatctacttgcacactctcccattatatttattgtttattttaggtcaaacccaagttagttcaatcctgattcatatttaatttgaagatagaaggtttgcagatattctctagtcatttgttaatatttcttcgtgatgacatatcacatgtcagccactgtgatagaggctgaggaatccaagaaaatggccagtaagatcaatgtgacggcagggaaatgtatgtgtgtctattttgtaactgtaaagatgaatgtcagttgttatttattgaaatgatttcacagtgtgtggtcaacatttctcatgttgaagctttaagaactaaaatgttctaaatatcccttggcattttatgtctttcttgtaagatactgccttgtttaatgttaattatgcagtgtttccctctgtgttagagcagagaggtttcgatatttattgatgttttcacaaagaacaggaaaataaaatatttaaaaatat

SEQ ID NO: 48 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTREVTVNVLSPRYEIVIITVVAAAVIMGTAGLSTYLYNRQRKIKKYRLQQAQKGTPMKPNTQATPP

SEQ ID NO: 49 - DNA, 2986 bp, Homo sapiens
gcgccccagtcgacgctgagctcctctgctactcagagttgcaacctcagcctcgctatggctcccagcagcccccggcccgcgctgcccgcactcctggtcctgctcggggctctgttcccaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcgaggtgaccgtgaatgtgctctccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctgaacctatcccgggacagggcctcttcctcggccttcccatattggtggcagtggtgccacactgaacagagtggaagacatatgccatgcagctacacctaccggccctgggacgccggaggacagggcattgtcctcagtcagatacaacagcatttggggccatggtacctgcacacctaaaacactaggccacgcatctgatctgtagtcacatgactaagccaagaggaaggagcaagactcaagacatgattgatggatgttaaagtctagcctgatgagaggggaagtggtgggggagacatagccccaccatgaggacatacaactgggaaatactgaaacttgctgcctattgggtatgctgaggcccacagacttacagaagaagtggccctccatagacatgtgtagcatcaaaacacaaaggcccacacttcctgacggatgccagcttgggcactgctgtctactgaccccaacccttgatgatatgtatttattcatttgttattttaccagctatttattgagtgtcttttatgtaggctaaatgaacataggtctctggcctcacggagctcccagtccatgtcacattcaaggtcaccaggtacagttgtacaggttgtacactgcaggagagtgcctggcaaaaagatcaaatggggctgggacttctcattggccaacctgcctttccccagaaggagtgatttttctatcggcacaaaagcactatatggactggtaatggttcacaggttcagagattacccagtgaggccttattcctcccttccccccaaaactgacacctttgttagccacctccccacccacatacatttctgccagtgttcacaatgacactcagcggtcatgtctggacatgagtgcccagggaatatgcccaagctatgccttgtcctcttgtcctgtttgcatttcactgggagcttgcactattgcagctccagtttcctgcagtgatcagggtcctgcaagcagtggggaagggggccaaggtattggaggactccctcccagctttggaagggtcatccgcgtgtgtgtgtgtgtgtatgtgtagacaagctctcgctctgtcacccaggctggagtgcagtggtgcaatcatggttcactgcagtcttgaccttttgggctcaagtgatcctcccacctcagcctcctgagtagctgggaccataggctcacaacaccacacctggcaaatttgattttttttttttttttcagagacggggtctcgcaacattgcccagacttcctttgtgttagttaataaagctttctcaactgcc